(19) United States
(12) Patent Application Publication
(10) Pub. No.: US 2002/0150961 A1
(43) Pub. Date: Oct. 17, 2002

(54) Activity-dependent cysteine protease profiling reagent

(75) Inventors: Matthew Bogyo, Mill Valley, IA (US); Doron Greenbaum, San Francisco, CA (US)

Correspondence Address: LAW OFFICES OF JONATHAN ALAN QUINE, P O BOX 458, ALAMEDA, CA, 94501

(73) Assignee: The Regents of the University of California Office of Technology Management

(21) Appl. No.: 10/035,451

(22) Filed: Nov. 8, 2001

Related U.S. Application Data
(60) Provisional application No. 60/266,295, filed on Nov. 10, 2000.
(60) Provisional application No. 60/287,993, filed on May 1, 2001.
(60) Provisional application No. 60/308,905, filed on Jul. 30, 2001.
(60) Provisional application No. 60/315,117, filed on Aug. 27, 2001.

(51) Int. Cl. C12Q 1/34; C12Q 1/37; C07K 14/00

(52) U.S. Cl. 435/23; 530/350

(57) ABSTRACT

Probes are provided having specificity for papain cysteine hydrolases comprising an electrophile, exemplified by an epoxide, a hydrophobic group for fitting into the hydrolase pocket and a moiety that provides for detection and/or isolation. A variety of compound having hydrophobic side chains from an oligopeptide are exemplified using fluorescers, ligand members of specific binding pairs or radioactive labels for detection and/or isolation.

### CROSS-REFERENCE TO RELATED APPLICATIONS

This application claims priority to U.S. Ser. No. 60/266,295, filed on Nov. 10, 2000, to U.S. Ser. No. 60/287,993, filed on May 1, 2001, and to U.S. Ser. No. 60/308,905, filed on Jul. 30, 2001, and to U.S. Ser. No. 60/315,117, filed on Aug. 27, 2001, all of which are incorporated herein by reference in their entirety for all purposes.

### STATEMENT AS TO RIGHTS TO INVENTIONS MADE UNDER FEDERALLY SPONSORED RESEARCH AND DEVELOPMENT

[Not Applicable]

### FIELD OF THE INVENTION

This invention pertains to the field of proteomics. In particular, this invention provides novel probes that are useful for profiling cysteine hydrolase activity, for screening for selective inhibitors of various cysteine hydrolases, and for inhibiting various cysteine hydrolases.

### BACKGROUND OF THE INVENTION

Various approaches for studying global cellular processes permit the analysis of differential changes within large sets of known and unknown genes or proteins. DNA microarray techniques allow analysis of genome-wide changes in mRNA transcription for a given cellular stimulus (Schena et al. (1998) Trends in Biotechnology 16: 301-306; DeRisi and Iyer (1999) Curr. Opin. Oncol. 11: 76-79. Advances in 2D gel electrophoresis coupled to highly sensitive mass spectrometry techniques now allow the rapid identification of proteins from whole cells or tissue extracts (Jungblut et al. (1999) Electrophoresis 20: 2100-2110; Celis et al. (1998) Febs Letts., 430: 64-72). While these techniques have revolutionized the global analysis of biological processes, often information about function of enzymatic proteins can only be inferred by analysis of transcriptional/translational co-regulation of sets of genes under different stimuli. However, levels of transcription and translation of an enzyme, in many cases, do not correlate with its activity (Gygi et al. (1999) Molecular and Cellular Biology 19: 1720-1730).

To assign function to enzymatic proteins on a genome-wide scale, a method to obtain direct information about enzymatic activity is necessary. Since the simultaneous targeting of all enzyme classes with a single probe is likely to be impossible, the present invention focuses on typically proteolytic enzymes, in particular the cysteine hydrolases.

In particular, the papaine family of cysteine proteases serves as a good model system for several reasons. Firstly, most cysteine proteases are synthesized with an inhibitory propeptide that must be proteolytically removed to activate the enzyme (Cygler, et al. (1996) Structure 4: 405-416; Coulombe et al. (1996) EMBO J. 15: 5492-503) resulting in expression profiles that do not directly correlate with activity. Secondly, the largest set of papaine-like cysteine proteases, the cathepsins, act in concert to digest a protein substrate. Thus, information regarding regulation of activity of each member relative to one another is critical for understanding their collective function. Furthermore, the cathepsins are involved in many critical biological processes, and biochemical studies of function have been limited to family members that have been cloned and expressed or purified from crude tissue. Finally, a large body of information is available regarding covalent, suicide substrate inhibitors that specifically target this family of cysteine proteases.

The papaine family is classified into several major groups, most notable of which are the bleomycin hydrolases, calpains, caspases, and cathepsins. To date, 14 human cathepsins have been cloned and sequenced. Several of these proteases are key players in normal physiological processes such as antigen presentation (Villadangos et al. (1999 Immun. Rev., 172: 109-120), bone remodeling (Gelb et al. (1996) Science 273: 1236-1238) and prohormone processing (Beinfeld (1998) Endocrine 8: 1-5). In addition, several of these proteases are involved in pathological processes such as rheumatoid arthritis (Iwata et al. (1997) Arthritis and Rheumatism, 40: 499-509), cancer invasion and metastasis (Yan et al. (1998) Biol. Chem., 379: 113-123) and Alzheimer's disease (Golde et al. (1992) [see comments] Science 255: 728-730; Munger et al. (1995) Biochem. J, 311: 299-305).

The enzymatic mechanism used by the papaine family of proteases has been well studied and is highly conserved. Thus, electrophilic substrate analogs that are only reactive in the context of this conserved active site can be used as general probes of function. A wide range of electrophiles have been developed as mechanism-based, cysteine protease inhibitors including diazomethyl ketones (Shaw, E. (1994) Meth. Enzym., 244: 649-656), fluoromethyl ketones (Shaw et al. (1986). Biomedica Biochimica Acta 45: 1397-1403), acyloxymethyl ketones (Pliura et al. (1992) Biochem. J, 288: 759-762), O-acylhydroxylamines (Brömme et al. (1989) Biochem. J., 263: 861-866), vinyl sulfones (Palmer et al. (1995) J. Med. Chem., 38: 3193-3196), and epoxysuccinic derivatives (Barrett and Hanada (1982) Biochem. J., 201: 189-198). These inhibitors typically consist of a peptide specificity determinant attached to an electrophile that becomes irreversibly alkylated when bound in close proximity to an attacking nucleophile.

Several groups have recognized the value of using irreversible mechanism-based inhibitors as affinity labels (Rauber et al. (1988) Analyt. Biochem., 168: 259-264; Bogyo et al. (1998) Chem Biol., 5: 307-320; Bogyo et al. (2000) Chem Biol., 7: 27-38; Mason et al. (1989) Biochem. J. 257: 125-129; Mason et al. (1989) Biochem. J. 263: 945-949).

Similar affinity labeling approaches have been used extensively to study or identify proteases such as the proteasome (Bogyo et al. (1998) Chem Biol., 5: 307-320; Bogyo et al. (1997) Proc. Natl. Acad. Sci., USA, 94: 6629-6634; Meng et al. (1999) Proc. Natl. Acad. Sci., USA, 96: 10403-10408), caspases (Faleiro et al. (1997) EMBO J. 16: 2271-2281; Nicholson and Lazebnik (1995) Nature 376: 37-43), cathepsins (Bogyo et al. (2000) Chem Biol., 7: 27-38; Mason et al. (1989) Biochem. J. 263: 945-949), and methionine amino peptidase (Griffith and Liu (1997) Chem Biol., 4: 461-471; Sin, et al. (1997) Proc. Natl. Acad. Sci., USA, 94: 6099-6103). Cravatt and co-workers have taken advantage of the broad class-specific reactivity of fluorophosphonates towards serine proteases (Liu et al. (1999) Proc. Natl. Acad. Sci., USA, 96: 14694-14699). By incorporation of a simple, extended alkyl chain capped with a biotin moiety, they have created a broad serine protease-specific probe (FP-Biotin) for functional proteomic analysis of serine proteases in cells and/or crude cellular extracts.

There is interest in developing specific compounds that have narrow or broad range specificity for target cysteine enzymes. These compounds can not only serve to identify their target enzymes in cells, particularly where the cells are associated with particular indications, but may also serve to map the surface of the target site of the target enzymes, where variations in structure and polarity can serve to develop compounds that may serve as reversible or irreversible inhibitors of the target enzymes.

### SUMMARY OF THE INVENTION

This invention provides functional proteomics tools that can be used to determine global patterns of activity for cysteine proteases, especially the papaine family of cysteine proteases. In particular, compounds (e.g., probes) that specifically bind to cysteine proteases are provided. Preferred compounds of this invention comprise a specificity determining group bound to electrophile active group that reacts at the active site of the target enzyme (e.g. cysteine hydrolase). Preferred compounds additionally comprise a group that imparts a desirable functionality (e.g. a detectable signal) to the compound.

Particularly preferred, probes comprising epoxides, usually of a defined stereochemistry, are employed linked to a hydrophobic moiety that fits into or otherwise interacts with the active site of the target cysteine protease. Contact of the probe to the target cysteine protease results in covalent bonding of the probe to the enzyme. A variety of different hydrophobic groups are found to vary the specificity and the particular enzyme to which the probe binds. Certain preferred compounds (probes) of this invention are illustrated herein by formulas I-XI.

In certain embodiments, this invention expressly excludes DCG-04 and/or DCG-03. In addition, or alternatively, this invention can expressly exclude all (e.g., 19) members of the probe library described in Example 1. In certain embodiments, the invention also expressly excludes any one or more or all probes described in Greenbaum et al. (2000) Chem. Biol., 7(8): 569-581; Shaw (1994) Meth. Enzym., 244: 649-656), Shaw et al. (1986). Biomedica Biochimica Acta 45: 1397-1403, Pliura et al. (1992) Biochem. J, 288: 759-762, Brömme et al. (1989) Biochem. J., 263: 861-866), Palmer et al. (1995) J. Med. Chem., 38: 3193-3196, Barrett and Hanada (1982) Biochem. J., 201: 189-198, Rauber et al. (1988) Analyt. Biochem., 168: 259-264; Bogyo et al. (1998) Chem Biol., 5: 307-320; Bogyo et al. (2000) Chem Biol., 7: 27-38; Mason et al (1989) Biochem. J 257: 125-129; Mason et al. (1989) Biochem. J. 263: 945-949, Bogyo et al. (1997) Proc. Natl. Acad. Sci., USA, 94: 6629-6634; Meng et al. (1999) Proc. Natl. Acad. Sci., USA, 96: 10403-10408, Faleiro et al. (1997) EMBO J. 16: 2271-2281; Nicholson and Lazebnik (1995) Nature 376: 37-43; Bogyo et al. (2000) Chem Biol., 7: 27-38; Mason et al. (1989) Biochem. J. 263: 945-949; Griffith and Liu (1997) Chem Biol., 4: 461-471; Sin, et al. (1997) Proc. Natl. Acad. Sci., USA, 94: 6099-6103; Liu et al. (1999) Proc. Natl. Acad. Sci., USA, 96: 14694-14699; and/or Hawthorne et al. (1998) Anal. Biochem., 261: 131-138.

The compounds of this invention to provide means for profiling cells for the active cysteine proteases being expressed, and means to screen for and/or to design specific drugs as inhibitors. The compounds of this invention can be used with combinatorial libraries may be used to compounds with different specificities for various target cysteine proteases.

These compounds of this invention provide functional information that can be used in concert with existing genomic and proteomic methods to correlate gene and protein expression profiles with enzymatic activity. Furthermore, diversification of core compounds using solid-phase combinatorial chemistry provides libraries of compounds that can be used to obtain information about inhibitor specificities of targeted protease. This information is of use in the generation of selective inhibitors without the need for prior characterization and purification of protease targets. Addition of a reporter function, such as a radioactive iodine, to inhibitors permits the visualization of covalently modified proteases in a standard SDS-PAGE gel format. Labeling intensity provides a read-out of relative enzymatic activity. Furthermore, both known and novel proteases are targets for analysis by this methodology.

### DEFINITIONS

The terms “polypeptide”, “oligopeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The term also includes variants on the traditional peptide linkage joining the amino acids making up the polypeptide.

The term “residue” or “amino acid” as used herein The term “residue” as used herein refers to natural, synthetic, or modified amino acids. Various amino acid analogues include, but are not limited to, 2-aminoadipic acid, 3-aminoadipic acid, beta-alanine, beta-aminopropionic acid, 2-aminobutyric acid, 4-aminobutyric acid, piperidinic acid, 6-aminocaproic acid, 2-aminoheptanoic acid, 2-aminoisobutyric acid, 3-aminoisobutyric acid, 2-aminopimelic acid, 2,4- diaminobutyric acid, desmosine, 2,2′-diaminopimelic acid, 2,3-diaminopropionic acid, N-ethylglycine, N-ethylasparagine, hydroxylysine, allo-hydroxylysine, 3-hydroxyproline, 4-hydroxyproline, iIsodesmosine, allo-isoleucine, N-methylglycine, sarcosine, N-methylisoleucine, 6-N-methyllysine, norvaline, norleucine, ornithine, etc.

The term “cysteine hydrolases” is used herein consistently with conventional usage of those of skill in the art. The family of cysteine proteases is characterized in a number of publications known to those of skill in the art (see, e.g., Rawlings and Barrett, (1994) Meth. Enzymology, 224: 461-486, Academic Press, S.D.).

The “papaine protease family” refers to a family of serine hydrolases based on structural homology to enzymes including papaine.

As used herein, an “antibody” refers to a protein or glycoprotein consisting of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively.

Antibodies exist as intact immunoglobulins or as a number of well characterized fragments produced by digestion with various peptidases. Thus, for example, pepsin digests an antibody below (i.e. toward the Fc domain) the disulfide linkages in the hinge region to produce F(ab)′2, a dimer of Fab which itself is a light chain joined to V_{H}-C_{H}1 by a disulfide bond. The F(ab)′2 may be reduced under mild conditions to break the disulfide linkage in the hinge region thereby converting the (Fab′)2 dimer into an Fab′ monomer. The Fab′ monomer is essentially a Fab with part of the hinge region (see, Paul (1993) Fundamental Immunology, Raven Press, N.Y. for a more detailed description of other antibody fragments). While various antibody fragments are defined in terms of the digestion of an intact antibody, one of skill will appreciate that such fragments may be synthesized de novo either chemically, by utilizing recombinant DNA methodology, or by “phage display” methods (see, e.g., Vaughan et al. (1996) Nature Biotechnology, 14(3): 309-314, and PCT/US96/10287). Preferred antibodies include single chain antibodies, e.g., single chain Fv (scFv) antibodies in which a variable heavy and a variable light chain are joined together (directly or through a peptide linker) to form a continuous polypeptide.

The term “probe” refers to a molecule that specifically binds to a target molecule (preferably a cysteine hydrolase) and provides a detectable signal or tag that can be used to detect and/or quantify the target molecule. The term probe can also refer to the probe molecule in combination with other reagents, e.g. a buffer system. The particular usage will be clear from the context.

“probe library” refers to a collection of different probes, preferably a collection of different probes having the structure represented in formula I. The probe library comprises at least 2, preferably at least 4, more preferably at least 10, most preferably at least 19 different probes. Certain larger libraries comprise at least 20, preferably at least 50, more preferably at least 100, and most preferably at least 1000, or at least 4,000 different probes.

The phrase “modulate the activity” when used in reference to an enzyme (e.g. a cysteine hydrolase) refers to increasing or decreasing the activity of the enzyme. The increase or decrease can be effected by direct interactions between the enzyme and a “modulating agent” and/or by indirect interactions, e.g. with cofactors, or other components in a pathway that effects activity of the enzyme. The increase or decrease can also be by an increase or decrease in transcription and/or translation of the enzyme.

An “electrophile” refers to a chemical compound or group that is attracted to electrons and/or tends to accept electrons, particularly when in the presence of an “electron-rich” species.

The term “specific binding” when used with respect to a probe of this invention refers to binding of a target protein by a probe where the binding is diminished or lost when the target protein is denatured (e.g. heat denatured). Thus, in preferred embodiments, specific binding is a function of the secondary and/or tertiary structure of the target protein. The binding is regarded as “diminished” where there is a difference between the binding of the probe to the undenatured protein and the binding of the probe to the denatured protein is measurable, and preferably where the difference is statistically significant (e.g. at greater than 80%, preferably greater than about 90%, more preferably greater than about 98%, and most preferably greater than about 99% confidence level). Particularly preferred embodiments, specific binding shows a at least a 1.2 fold, preferably at least a 1.5 fold, more preferably at least a 2 fold, and most preferably at least a 4 fold or even a 10-fold difference from the denatured protein. In a most preferred embodiment, binding of the probe to a denatured protein sample is essentially indistinguishable from the background signal.

“binding profile” or a “specificity fingerprint” is a pattern of binding of one or more probes of this invention to a biological sample or to a component of a biological sample.

The term “ligand” refers to functional group, atom, or molecule that is attached to another atom or molecule (e.g., in this case the probe) that can combine with and thereby bind to another substance.

An “affinity tag” refers to a molecule or domain of a molecule that is specifically recognized and bound by another molecule (i.e. a cognate binding partner). Examples of affinity tags include, but are not limited to biotin, avidin, streptavidin, Ni-NTA, His₆, and the like.

An “epitope tag” refers to a molecule or domain of a molecule that is specifically recognized by an antibody. However, the term “epitope tag” can be used more broadly to also include a molecule or domain of a molecule bound by a binding partner (ligand) other than an antibody. In this instance the terms “epitope tag” and “affinity tag” are similar. Thus, for example, in addition to epitopes recognized in epitope/antibody interactions, epitope tags can also comprise “epitopes” recognized by other binding molecules (e.g. ligands bound by receptors), ligands bound by other ligands to form heterodimers or homodimers, His₆ bound by Ni-NTA, and the like.

The terms “linker” and “spacer” are used interchangeably. A wide variety of linkers (spacers) are suitable for use in the probes of this invention. Such linkers include, but are not limited to straight or branched-chain carbon linkers, heterocyclic carbon linkers, and the like. Preferred linkers are C₁ to C₂₀, more preferably C₂ to C₁₀, and most preferably C₃ to C₆ straight chain carbon linkers. Particularly preferred linkers include, but are not limited to straight chain saturated alkyl amino acids such as amino hexanoic acid, as well as spacers greater or fewer methylene groups (e.g. between 2 and 10 methylene groups). The linkers can also include various cleavable linkers that can be used to selectively release probe-modified peptides. A number of different cleavable linkers are known to those of skill in the art (see, e.g., U.S. Pat. Nos. 4,618,492, 4,542,225, and 4,625,014). The mechanisms for release of an agent from these linker groups include, for example, irradiation of a photolabile bond and acid-catalyzed hydrolysis. One particularly preferred linker is a photolabile linker (PhotoRelease™) that can be used to selectively release probe-modified peptides by UV irradiation. This linker is commercially available from Advanced Chemtech. Also a free amino lysine reside can be used as a spacer in place of the lysine-biotin conjugate that can be covalently attached to affigel (BioRad) to create an affinity resin. The linker can also include a 1-2-diol moiety that could be cleaved by mild oxidation with sodium periodate to specifically release peptide products from affigel or from streptavidin agarose. Representative oxidizable cleavable linkers are illustrated in FIG. 11 and various photolabile cleavable linkers are illustrated in FIG. 12.

The term “small organic molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

The term “modified streptavidin” refers to a monomeric avidin or streptavidin or to a derivatized streptavidin or to a streptavidin analog. Certain modified streptavidins show reduced affinity to biotin.

The term “biological sample”, as used herein, refers to a sample obtained from an organism, from components (e.g., cells or tissues) of an organism, and/or from in vitro cell or tissue cultures. The sample may be of any biological tissue or fluid (e.g. blood, serum, lymph, cerebrospinal fluid, urine, sputum, etc.). Biological samples may also include organs or sections of tissues such as frozen sections taken for histological purposes.

The term “crude cellular extract” refers to a relatively unpurified or completely unpurified derivative obtained from one or more cells. A typical crude cellular extract is simply a suspension of homogenized cells. Certain crude cellular extracts include cellular extracts that have been filtered, centrifuged, or otherwise treated to remove particulate matter.

### BRIEF DESCRIPTION OF THE DRAWINGS

FIG. 1 illustrates the structure of epoxide inhibitors and probes E-64, JPM-565 and DCG-04. Preferred radiolabel attachment and affinity sites are indicated for each compound.

FIGS. 2A and 2B illustrate the synthesis of DCG-04. FIG. 2A illustrates the epoxy acid building block (epoxide (I)) and FIG. 2B illustrates a solid-phase synthesis scheme for DCG-04. Details of the synthesis and characterization of peptide epoxides can be found herein in Example 1.

FIGS. 3A and 3B illustrate DCG-03 and DCG-04 labeling of active proteases in dendritic cell extracts. FIG. 3A: Total cell extracts from DC2.4 cells were diluted into either pH 5.5 or pH 7.4 buffer, preheated to 100° C. for 1 min (+preheating) or not (−preheating) and labeled with 50 μM DCG-03 and DCG-04. Samples were separated by SDS-PAGE (12.5% gel) and labeled bands visualized by affinity blotting as described in the experimental section. FIG. 3B: Same as for FIG. 3A except ¹²⁵-I labeled versions of DCG-03 and DCG-04 were used and the gels were analyzed by autoradiography. The location of cathepsin B, L, and S are indicated for reference based on their known molecular weights.

FIGS. 4A and 4B show that DCG-03 and DCG-04 target the same polypeptides as the parent compounds E-64 and JPM-565. FIG. 4A: Total cellular extracts from DC2.4 cells were incubated with increasing concentrations of E-64 as indicated for 30 min at 25° C. followed by addition of 50 μM DCG-04 and further incubation for 1 hr. Samples were resolved by SDS-PAGE (12.5%) and labeled bands visualized by affinity blotting. FIG. 4B: Total cellular extracts were labeled with either ¹²⁵I-labeled forms (auto-rad) or with non- labeled forms (blot) of DCG-03, DCG-04, and JPM-565 followed by separation by SDS-PAGE (12.5%) and analysis as indicated. The location of cathepsin B and S are indicated for reference based on their known molecular weights.

FIGS. 5A and 5B illustrate activity profiling across a disease progression. Tissue culture cells were isolated from carcinomas generated by application of a chemical mutagen to the skin of mice (see Example 1). Progression begins at the left with the non invasive benign cells (C5N and P6) and progresses to the right through papilloma cell lines (PDV and PDV-C57), squamous cell carcinomas (B9, A5, and D3), and finally highly invasive spindle cell carcinomas (Car B and Car C). Total cellular lysates were normalized with respect to protein concentration and labeled with ¹²⁵I-DCG-04 (FIG. 5A) and the cathepsin B-specific probe ¹²⁵I-MB-074 (FIG. 5B). A pre-heat control from the C5N lysate was included in A) to show background labeling.

FIG. 6 illustrates profiling protease inhibitor specificity. Lysates from the dendritic cell line DC2.4 (panels A and B) or purified cathepsin H (panel C) were preincubated with 50 μM of each of the 19 derivatives of DCG-04 and then labeled with ¹²⁵I-DCG-04 (panels A and C) or ¹²⁵I-MB-074 (panel B) as indicated. The general structure of the inhibitors is shown with the variable amino acid sidechain indicated as an X (competitor; top). The predominant labeled polypeptides in A) are labeled with numbers and positions of cathepsin B and S are indicated for reference.

FIG. 7 shows activity profiling of cysteine proteases across tissue types. Labeling of total cellular extracts (100 μg protein/lane) from rat brain, kidney, liver, prostate, and testis with ¹²⁵I-DCG-04 at pH 5.5. Samples were analyzed by SDS-PAGE followed by autoradiography. A pre-heating control was included for each tissue type to indicated background labeling.

FIG. 8 illustrates affinity purification of DCG-04 targeted proteases from rat kidney. Panel A illustrates labeling of total cellular extracts (100 μg protein/lane) from rat kidney with 50 μM DCG-04 at pH 5.5. Samples were analyzed by SDS-PAGE followed by affinity blot. Panel B shows the results of anion exchange chromatography of rat kidney lysate using a gradient from 0.05-1M NaCl, pH 9.0. Fractions were analyzed by addition of DCG-04 (50 μM) followed by SDS-PAGE and affinity blotting. Fractions containing DCG-04 labeled proteins were pooled (fractions 5-7 and Fractions 11-13). Panel C: Pooled fractions were labeled with DCG-04 (50 μM), and DCG-04 modified proteins bound to a monomeric-avidin column, washed with 1M NaCl, and eluted using 2 mM biotin. A sample of material from pools prior to application to the affinity column (PC) along with column flow through (FT) and biotin elution fractions (E1-E5) were analyzed by SDS-PAGE followed by silver staining. Panel D: Elutions containing labeled proteins were pooled, volumes reduced, and analyzed by 2D IEF electrophoresis followed by silver staining. Spots labeled with numbers were excised and used for sequencing.

FIG. 9 shows a low energy CID spectrum of tryptic peptides with MH⁺=1429.7. The doubly charged ion at m/z 715.35 was selected as a precursor ion. Only the C-terminal fragment ions used for sequence determination are labeled.

FIG. 10 shows certain preferred probes of this invention.

FIG. 11 shows various cleavable (oxidizable) linkers.

FIG. 12 shows various cleavable (photolabile) linkers.

FIG. 13 shows structures of fluorescent DCG-04 probes. The four non-overlapping fluorescent DCG-04 analogs include BODIPY588/616-DCG-04 (Red-DCG-04), BODIPY493/503-DCG-04 (Blue-DCG-04), BODIPY530/550-DCG-04 (Green-DCG-04), and BODIPY558/568-DCG-04 (Yellow-DCG-04). These probes are synthesized from the corresponding DCG-04 free amine by reaction with the corresponding BODIPY succinamide ester. All fluorophores were purchased from Molecular Probes.

FIGS. 14A and 14B illustrate affinity labeling of papain family proteases using fluorescent ABPs. FIG. 14A: Purified cathepsins (as indicated) were diluted into pH 5.5 buffer and labeled with 100 nM Yellow-DCG-04, Red-DCG-04, Green-DCG-04 or Blue-DCG-04 for 1 hour. Samples were separated on a 15% SDS-PAGE gel and labeled bands visualized using an ABI 377 DNA sequencer as described in Example 2. FIG. 14B: Total cell extracts from rat liver were diluted into pH 5.5 buffer and labeled with 10 mM DCG-04, ¹²⁵I-DCG-04 (approx. 1×10⁶ CPM), or 100 nM red-, blue-, green-, and yellow-DCG-04. Samples were separated on a 15% SDS-PAGE gel and labeled bands were visualized (as indicated at bottom) by affinity blotting, autoradiography, or using a Molecular Dynamics Typhoon laser fluorescence scanner.

FIGS. 15A and 15B illustrate labeling of purified cathepsins with fluorescently labeled probes and localization of protease activity in situ. FIG. 15A: DC2.4 cells were grown in culture in serum-free media and treated overnight with Green-DCG-04 (1 mM final concentration) or FIG. 15B: pre-treated with 10 mM of E-64 for 1 hour and then labeled with 1 mM of Green-DCG-04. Fresh media was added and cells incubated for five hours to remove excess probe. Cells were visualized by fluorescence microscopy (Left panels) then collected, lysed in SDS sample buffer and analyzed by SDS-PAGE on an ABI 377 DNA sequencer (Right panels). Labeled proteases in the untreated cells are indicated with numbers. Note the complete competition of all protease species by E-64 pre-treatment.

FIGS. 16A, 16B, and 16C show the screening of peptide epoxide positional scanning libraries (PSLs). FIG. 16A: Structures of the general PSL scaffolds containing either (S,S) or (R,R) epoxides. PSLs contain a fixed P2 position (X) and P3 and P4 positions composed of an isokinetic mixture of 19 natural amino (all natural amino acids minus cysteine and methionine, plus norleucine; Mix). FIG. 16 B: Colorimetric cluster display of inhibition data. PSLs were used to profile purified cysteine proteases by pretreatment of samples with individual constant P2 libraries followed by labeling with ¹²⁵I-DCG-04. Labeling intensity of each target relative to the control untreated sample was used to generate percent competition values. These resulting data were clustered and visualized using programs designed for analysis of micro-array data (see Example 2). The tree structures at the top and left of the diagrams were obtained by hierarchical clustering and indicate the degree of similarity as a function of the height of the lines connecting profiles. FIG. 16C: Results from profiling proteases in rat liver extracts. Data was compiled and visualized as described in (FIG. 16B). Each constant non-natural amino acid is indicated with a number corresponding to its structures listed in the supplemental materials. Constant natural amino acids are indicted using the standard one letter code (n is used for norleucine). Natural amino acids attached to the R,R epoxide are indicated with “R,R”. Unknown protease bands in rat liver are numbered 1-4 and correspond to the bands shown in FIG. 14B. The color key is shown at the bottom.

FIG. 17A and 17B illustrate the profiling of changes in protease activity upon inhibitor treatment. Liver extracts (100 mg) were treated with 100 nM Red-DCG-04 or with 10 mM of Ac-XX-Q-(R,R)Eps library for 30 minutes and then 100 nM Blue-DCG-04. Reactions were quenched with IEF sample buffer and equal amounts of each reaction were co-loaded on a single IEF tube gel. Labeled proteins were separated on a 15% SDS PAGE and analyzed using an ABI 377 DNA sequencer. FIG. 17A, bottom panel shows the red and blue channels overlaid on a single image while the top and middle panels show the individual labeling profiles. Note the loss of activity of the circled protease upon inhibitor treatment. FIG. 17B: Active proteases in the liver extract were purified by a single step affinity purification of DCG-04-labeled liver extract. Silver-stained spots were excised and sequenced by LC-MS-TOF CID. The silver-stained spot corresponding to the labeled protease inhibited by Ac-XX-Q-(R,R)-Eps library was identified as cathespin B. Other papain family protease were also identified and are labeled with arrows.

FIGS. 18A and 18B illustrate the evaluation of specific protease inhibitors selected from library screening. Competition analysis of a negative control compound (YG(R,R)Eps), a cathepsin B-specific compound identified from the library screening (YQ-(R,R)Eps), and a previously described cathepsin B-specific inhibitor (MB-074). Several concentrations of each compound were incubated with 100 mg total liver extract for 30 minutes followed by labeling with ¹²⁵I-DCG-04 for 1 hour. (FIG. 18A: Inhibition dose response profiles for each compound. FIG. 18B: Direct labeling of 100 mg total liver extract with radioiodinated versions of DCG-04, MB-074 and YQ-(R,R,)Eps. Note the specificity of MB-074 and YQ-(R,R)Eps for cathepsin B.

FIG. 19 illustrates screening of small molecule libraries against the complete set of papain family cysteine proteases in Rat liver. This image shows a typical gel image generated from scanning of the gel as well as the process by which labeled bands can be quantitated (panel to left). Small molecules can be analyzed for their potency and selectivity for targets in the rat liver proteasome using this method. Note that each color data can be separately extracted due to non-overlapping emission spectra of the chosen fluorophores. This approach therefore allows analysis of up to 80 samples in a single gel using four color labels.

### DETAILED DESCRIPTION

Analysis of global changes in gene transcription and translation by systems-based genomics and proteomics approaches provides only indirect information about protein function. In many cases enzymatic activity fails to correlate with transcription or translation levels. Therefore, a direct method for broadly determining activities of an entire class of enzymes on a genome-wide scale is of great utility.

This invention provides a class of compounds that are useful functional proteomics tools. The compounds are generally specific for cysteine proteases and can be used to determine patterns of activity for cysteine hydrolases (e.g. the papaine family of cysteine proteases). These compounds provide functional information that can be used in concert with existing genomic and proteomic methods to correlate gene and protein expression profiles with enzymatic activity. Furthermore, diversification of the compound specificity determinants, e.g., using solid-phase combinatorial chemistry provides libraries of compounds that can be used to obtain information about inhibitor specificities of targeted cysteine hydrolases. This information is of use in the generation of selective inhibitors without the need for prior characterization and purification of hydrolase/protease targets.

The compounds can be used to specifically bind to and thereby identify cysteine protease activity even in a complex biological mixture, such as a cellular cytosol or lysate.

The compounds of this invention bind to and thereby covalently modify their target protease(s). They can be used to rapidly identify and/or isolate targets (e.g. novel proteases). Other uses of the compounds of this invention include, but are not limited to the profiling of cysteine hydrolase activity in disease states, the analysis of selectivity of various small molecules and drugs, and the diagnostic tracking of cysteine proteases in various biological samples (e.g. whole cells, cell lysates, biological fluids, and biopsied tissue samples).

I. Probe Structure

In preferred embodiments, the compounds of this invention comprise reactive electrophiles joined to a hydrophobic moiety that provides affinity/specificity for individual cysteine proteases or ranges (classes) of cysteine proteases. Besides the two elements of the compounds indicated above, other groups may be added to the hydrophobic moiety without interference with the specificity, while providing for other attributes, such as identification, isolation, solubility, interaction with other compounds, etc.

Besides the two elements of the compounds indicated above, other groups may be added to the hydrophobic moiety without interference with the specificity, while providing for other attributes, such as identification, isolation, solubility, interaction with other compounds, etc.

Depending upon the intended use of the compounds of this invention, the compounds can be non-labeled, labeled with a detectable label, tagged with a ligand for which a binding partner is available, joined to an effector that provides a particular enzymatic and/or cayalytic activity, and the like. In this way, depending on the tag, label, effector, etc., one can detect the presence of the compound at a site (e.g. in a cell), isolate the compound, separate the reaction product of the compounds, deliver a particular catalytic or enzymatic activity to a particular location in a cell, and the like.

In certain preferred embodiments, the compounds or probes of this invention will have the following formula:

A-L¹-Hy-L²-E I.

where:

A can be any group, usually of at least about 15 Dal and usually not more than about 2 kDal, more usually not more than about 1 kDal, that does not interfere with the bonding of the compound to the target cysteine proteases and that imparts a desirable function to the compound (e.g. a detectable label, a ligand, etc.);

L¹ and L² can be the same or different and each can be a bond, a chain of from 1 to 40, usually 1 to 30 atoms, and will usually have from 0 to 36, more usually from 0 to 30 carbon atoms and from 0 to 12, usually from 0 to 8 heteroatoms, that are nitrogen, oxygen, phosphorous and sulfur, being amines, carboxy derivatives, such as amides and esters, ethers (including thioethers), and the like;

Hy is a hydrophobic group that binds with specificity to the binding site of the cysteine protease, preferably to the S2 pocket of the cysteine hydrolase, providing specificity to the compound for bonding to cysteine proteases, the hydrophobic group varying with the range of specificity desired for the compound, where the hydrophobic group will usually be of at least about 5 carbon atoms, usually at least about 6 carbon atoms and not more than about 50 carbon atoms, usually not more than about 36 carbon atoms, and may be aliphatic, alicyclic, aromatic or heterocyclic, or combinations thereof, and will have from 1 to 6, usually 1 to 4 heteroatoms, that are oxygen, nitrogen, sulfur, halogen, phosphorous, etc.; and

E is an electrophile that is active at the active site of the cysteine hydrolase to form a covalent bond at the . In certain preferred embodiments, the electrophile is one that is typically inert, but becomes reactive when around electron-rich species (e.g. when localized in or near the binding site of a cysteine hydrolase). In particularly preferred embodiments, electrophile includes, but is not limited to various ketones (e.g. diazomethyl ketone, fluoromethyl ketone, acyloxymethyl ketone, chloromethyl ketone, etc.), epoxides, particularly carboxy- substituted epoxides, reactive α-substituted methyl keto carbonyls, e.g. halo, diazo, and acyloxy, vinyl sulfones, O-acyl hydroxylamines, etc. In certain embodiments, E will comprise at least 2 carbon atoms and not more than about 12 carbon atoms, usually not more than about 8 carbon atoms. In certain embodiments, E will comprise at least one heteroatom and usually not more than 6 heteroatoms, where preferred heteroatoms include nitrogen, oxygen, sulfur and phosphorous.

Particularly preferred electrophiles, when coupled to the peptide specificity determinant (e.g. the hydrophobic group), form a “suicide substrate”, that is, a substrate that binds essentially irreversibly with its “target” cysteine protease (e.g. by forming a covalent linkage to the target). The suitability of various electrophiles can therefore be readily assayed by coupling the electrophile to a particular substrate (e.g. -Tyr-Lys-), contacting that substrate to a target protease (e.g. a cathepsin), and determining whether the substrate/electrophile tightly (e.g. irreversibly) binds to the target protease.

A certain preferred electrophiles is an epoxide, particularly an epoxide having an activating group bonded to an annular carbon atom, particularly a carbonyl and more particularly a carboxy carbonyl. Preferably, both annular carbon atoms have activating groups. Where both annular carbon atoms are substituted, enantiomerically enhanced compositions will preferably be employed, such as R,R and S,S, substantially free of the other stereoisomer.

Compounds of this invention of particular interest have the following formula:

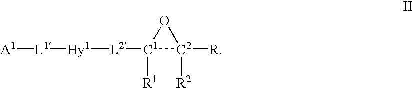

wherein:

A¹ is preferably moiety of from 1 to 30, usually from 4 to 20 carbon atoms and from 0 to 10, usually 0 to 8 heteroatoms, which include N, O, S, P and halo, that provides a detectable signal, e.g. a fluorescer, or a ligand for binding to a specific receptor or other cognate binding partner, where the complex of ligand and receptor allows for specific isolation, e.g. the ligand may be referred to as an affinity tag, or binding to another molecule of interest, e.g. an enzyme or functionalized protein; wherein when said moiety provides a detectable signal, said moiety will be carbocyclic or hetercyclic aromatic, generally having rings of from 5 to 7 annular atoms, where the rings may be fused or non-fused, and may be connected by a bond or chain of from 1 to 8 atoms, which may be saturated or unsaturated, generally the unsaturation will be ethylenic unsaturation;

L^{1′} and L^{2′} (e.g. linkers) are the same or different and are preferably an aliphatic chain of from 1 to 8, usually 1 to 6 carbon atoms joined to A^{1′} or the epoxide C¹ annular carbon atom and Hy¹ through the same or different functional group, which can be amino, amide, ester or ether (including thioether), where the chain may be substituted or unsubstituted, the total number of carbon atoms preferably being not more than 12 and there preferably being from 0 to 4 heteroatoms as described for L;

Hy¹ is a neutral, preferably hydrophobic, amino acid. Particularly preferred amino acids have at least 4, more preferably at least 5, still more preferably at least 6 carbon atoms, and generally not more than about 20 carbon atoms, usually not more than about 16 carbon atoms, that can be aliphatic, alicyclic, aromatic, or heterocyclic, branched or unbranched, aliphatically saturated or unsaturated, usually having not more than about 2 sites of unsaturation, ethylenic or acetylenic, where substituents on rings can be separated by 2, 3 or 4 annular members, the substituents normally being aliphatic groups of from 1 to 6 carbon atoms, halogen or nitrogen containing substituents, such as amino, including mono- and di-lower alkyl amino (lower alkyl is preferably of from 1 to 6, more preferably 1 to 3 carbon atoms), cyano, nitro, carboxamide, phosphoramide, and the like, there being from 1 to 3 rings, where the rings can be fused or unfused, and, when unfused, are usually separated by from 0 to 3 atoms, that is bonded together or having a bridge that will usually be alkylene, oxoalkylene, oxyalkylene, and the like; desirably there will be a side chain as the D or L stereoisomer;

the R groups are the same or different, in preferred embodiments there being not more than two of the R groups other than hydrogen, where the total number of carbon atoms for all of the R groups is from 0 to 8, usually from about 0 to 4; the R groups can include hydrogen, lower alkyl, e.g. of from 1 to 6, usually 1 to 3 carbon atoms, oxycarbonyl of from 1 to 3 carbon atoms, alkoxycarbonyl of from 2 to 5 carbon atoms, preferably being free of acidic groups; more preferably, R is alkoxycarbonyl and R²is hydrogen.

In certain preferred embodiments, the probes of this invention comprise a core amino acid or peptide recognition domain attached to the electrophile directly or through a linker (L). The probes also preferably include a ligand, affinity site or detectable label, and, in certain preferred embodiments, include a detectable label attached to the ligand or affinity site. Thus, in such particularly preferred embodiments the probes can the formula:

A-L¹-(aa¹)ᵢ-(aa²)ⱼ-(aa³)ₖ-(aa⁴)ₗ-L²ₘ-E III

where A is a ligand, affinity tag, or detectable label, L¹ is a linker, L², when present, is a linker, aa¹, aa², aa³, and aa⁴, when present, are independently selected amino acids, i, j, k, l, and m are independently 0 or 1, E is an electrophile, and at least one of aa¹, aa², aa³, and aa⁴ are present.

As indicated above, “L” can be characterized as a bond or as a “linker” or “spacer” for joining the electrophile to the hydrophobic group (specificity determining group) and/or for joining the affinity tag ,ligand, label, etc. to the hydrophobic group (recognition domain). Generally a spacer or linker has no specific biological activity other than to join particular components of the probe or to preserve some minimum distance or other spatial relationship between them. However, the spacer may be selected to influence some property of the probe such as the folding, net charge, or hydrophobicity of the probe.

As indicated above, a wide variety of linkers (spacers) are suitable for use in the probes of this invention. Certain linkers include, but are not limited to straight or branched-chain carbon linkers, heterocyclic carbon linkers, and the like. Preferred linkers are C₁ to C₂₀, more preferably C₂ to C₁₀, and most preferably C₃ to C₆ straight chain carbon linkers. In one particularly preferred embodiment the linker is a hexanoic acid linker (e.g. an amino hexanoic acid linker).

Depending upon the desired specificity of the probe Hy (aa) will vary. In preferred embodiments, it will not have acidic groups, will be free of quaternary carbon atoms, the amino group to which the linking group is linked is preferably not an annular member; and the carboxy and amino are preferably not linked through a ring. Desirably, there is an aliphatic, alicyclic, aromatic side chain of from 2 to 16 carbon atoms, usually 3 to 16 carbon atoms and from 0 to 4 heteroatoms, preferably oxygen, nitrogen and sulfur, particularly at the α-carbon atom.

Hy can be a naturally occurring or unnatural amino acid, either D or L, where the amino group may be α to ω, usually be from about α to δ, preferably α; similarly the side chain may be at any site, but will come within the preferences for the amino group; usually Hy will be neutral or basic, preferably neutral, and may have amino, oxy or oxo substituents, e.g. keto and carboxy carbonyl; preferred groups include carbocyclic rings of from 5 to 7, usually 5 to 6 carbon atoms, there being from 1 to 3, usually 1 to 2 rings, which may be fused or unfused, aliphatic chains, branched or unbranched, saturated or unsaturated, usually having not more than 3 sites, usually not more than 2 sites of aliphatic unsaturation, either double or triple bonds. As the groups come within the above limitations, the probes are able to react with a number of different papain cysteine hydrolases. As one deviates from the reactive moieties, greater specificity is obtained and further deviations results in specificity with lower affinity or substantially no affinity. Furthermore, it appears that the R,R-stereoisomer and the S,S-stereoisomer provide for significant selectivity with the appropriate side groups.

As indicated above, suitable amino acids for incorporation into the probes of this invention include naturally occurring amino acids and modified or non-natural amino acids. Such modified amino acids include, but are not limited to, norleucine, episilon-aminocaproic acid, 4-aminobutanoic acid, tetrahydroisoquinoline-3-carboxylic acid, 8-aminocaprylic acid, 4-aminobutyric acid, α-aminoisobutyric acid, aminoisobuteric acid, aminobuteric acid, diethylglycine, α,β-dehydroaminobuteric acid, aminohexanoic acid, norvaline, T-butylglycine, 3-cyclohexyl-alanine, phenylglycine, α-cyclohexylglycine, 3-(1-naphthyl)-alanine, 3-(2-naphthyl)-alanine, 4-(boc-amino)-phenylalanine, biphenylalanine, 4-benzoyl-phenylalanine, homo-phenylalanine, α,β-dehydroleucine, α,β-dehydrovaline, 4-(aminomethyl)benzoic acid, 4-(aminomethyl) cyclohexane, 2-aminobenzoic acid, 3-aminobenzoic acid, (s)-2-amino-4-cyanobutyric acid, 4-methyl phenylalanine, p-nitrophenylalanine, pipecolic acid, isonipecotic, 1-trans-4-hydroxyproline, thiazolidine-4-carboxylic acid, (3s)-1,2,3,4-tetrahydroisoquinoline, 1-aminocyclopropane-1-carboxylic acid, 1-amino-1-cyclopentane-carboxylic acid, 1-amino-1-cyclohexane carboxylic acid, igl-oh, allylglycine, 3-amino-3-phenylpropionic acid, propargylglycine, (2-pyridyl)alanine, (2-furyl)-alanine, beta-styrylalanine, (2-thienyl)alanine, and the like. In certain preferred embodiments, non-natural amino acids are Fmoc-blocket.

Embodiments in which the peptide recognition domain of the probes of this invention is a monopeptide (i.e., i, j, and k are zero), or a dipeptide (i.e.. i and j are zero) show particularly good specificity for members of the papaine family of cysteine hydrolases. It is noted that, the group that binds to the S2 pocket (i.e. Hy) changes to this group (e.g. amino acid residue) are to have the greatest effect on specificity of the probes of this invention for a given target (e.g. a particular cysteine hydrolase).

Besides the variation at the affinity site (e.g. Hy, aaᵢ through aaₗ), there can be variation at the other terminus of the hydrophobic moiety. As indicated previously, these can be selected for a variety of purposes. There appears to be few restrictions as to what may be attached at this end, so there is a wide latitude in the groups employed for the various purposes. Generally speaking, as indicated previously primary purposes will be for isolation of the probe reaction product and detection of the probe reaction product. As many probes are able to pass through the cell membrane and react intracellularly, the subject probes can be used to follow the intracellular movement of the targets or determine their situs.

For the purpose of isolation and in some instances identification there will be present an affinity tag or a ligand. Suitable affinity tags or ligands include essentially any tag that can be bound by a cognate ligand or binding partner. Preferred affinity tags/ligands do not substantially interfere with binding of the probe to a target cysteine hydrolase.

Affinity tags are well known to those of skill in the art. Such tags include, but are not limited to biotin with avidin/streptavidin, ligands and their cognate receptors, particularly haptens and antibodies, polyhistidine with Ni-NTA, epitopes and cognate antibodies, and the like.

Certain affinity tags include epitope tags. Epitope tags are well known to those of skill in the art. Moreover, antibodies (intact and single chain) specific to a wide variety of epitope tags are commercially available. These include but are not limited to antibodies against the DYKDDDDK (SEQ ID NO:1) epitope, c-myc antibodies (available from Sigma, St. Louis), the HNK-1 carbohydrate epitope, the HA epitope, the HSV epitope, the HiS₄, HiS₅, and HiS₆ epitopes that are recognized by the His epitope specific antibodies (see, e.g., Qiagen), and the like.

In certain preferred embodiments, the ligand is tagged with a hexahistidine (His₆) epitope tag which is bound by a Cu, Ni, or Co complex. One particularly preferred complex for binding HiS₆ tags is Ni-NTA (Ni- nitrilotriacetic acid). In particularly preferred embodiments, the affinity tag is a biotin which can then be captured by avidin, streptavidin, or variants thereof.

In certain embodiments, e.g., for the purposes of detection and in some cases isolation, the compounds of this invention (probes) bear a detectable label. Virtually any detectable label can be used as long as it doesn't substantially interfere with the binding of the probe to its target cysteine hydrolase. Larger labels can be accommodated by the use of various linkers/spacers. Thus, other detectable labels suitable for use in the present invention include any composition detectable by spectroscopic, photochemical, biochemical, immunochemical, electrical, optical or chemical means. Such labels include, but are not limited to, fluorescent dyes (e.g., fluorescein, texas red, rhodamine), fluorescent proteins (green fluorescent protein (GFP), red fluorescent protein (RFP), and the like, see, e.g., Molecular Probes, Eugene, Oreg., USA), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold (e.g., gold particles in the 40 -80 nm diameter size range scatter green light with high efficiency), and the like. Patents teaching the use of such labels include U.S. Pat. Nos. 3,817,837; 3,850,752; 3,939,350; 3,996,345; 4,277,437; 4,275,149; and 4,366,241.

It will be recognized that fluorescent labels are not to be limited to single species organic molecules, but include inorganic molecules, multi-molecular mixtures of organic and/or inorganic molecules, crystals, heteropolymers, and the like. Thus, for example, CdSe-CdS core-shell nanocrystals enclosed in a silica shell can be easily derivatized for coupling to the molecules of this invention (Bruchez et al. (1998) Science, 281: 2013-2016). Similarly, highly fluorescent quantum dots (zinc sulfide-capped cadmium selenide) have been covalently coupled to biomolecules for use in ultrasensitive biological detection (Warren and Nie (1998) Science, 281: 2016-2018). Quantum dot fluorescent labels are commercially available from Quantum Dot Corporation, Hayward, Calif.

While in certain embodiments, “A” in formula I, above is a detectable label, other positions can also be labeled in the probe whether “A” is a label or another moiety. Thus, for example, in certain preferred embodiments, the probes are also labeled with a detectable label in addition to the ligand/affinity tag/label and thus provide dual-functionality probes. While, in such cases, the “second” detectable label is preferably a radioactive label (e.g. ³H, ¹²⁵I, ³⁵S, ¹⁴C, ³²P, etc.), it need not be so limited. The “second label” and the “other” labeling position, is chosen so as not interfere with the binding of the compound (probe) to a target cysteine protease. Labels (e.g., radioactive labels such as ³H, ¹²⁵I, ³⁵S, ¹⁴C, ³²P, etc.), can be attached in accordance with conventional means, for example, using tyrosine for labeling with a radioactive iodine (¹²⁵I).

Illustrative of the compounds of this invention employing various labels, having a an oligopeptide linker and using glutamic acid to link the label, are the following formulae, where E and L² have been defined previously and m is 0 when L² is a bond and is otherwise 1. Examples, of such embodiments, are illustrated by the probes of formula IV and V.

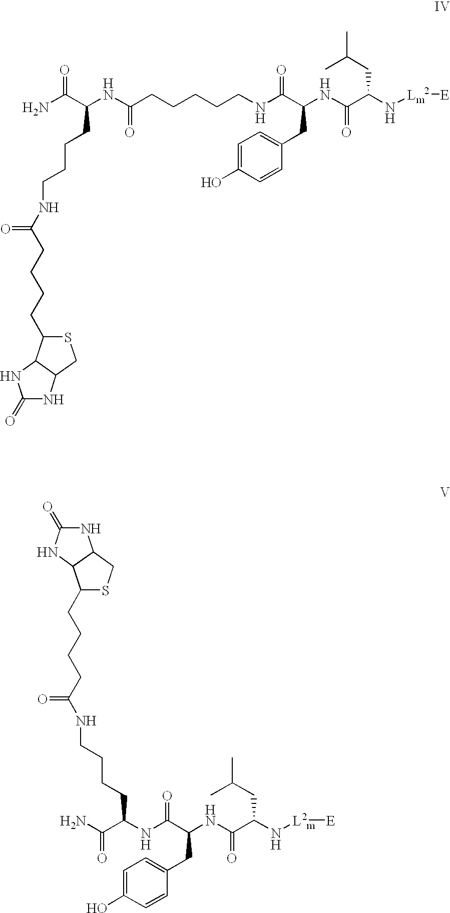

The following formula VI illustrates L² as a linker:

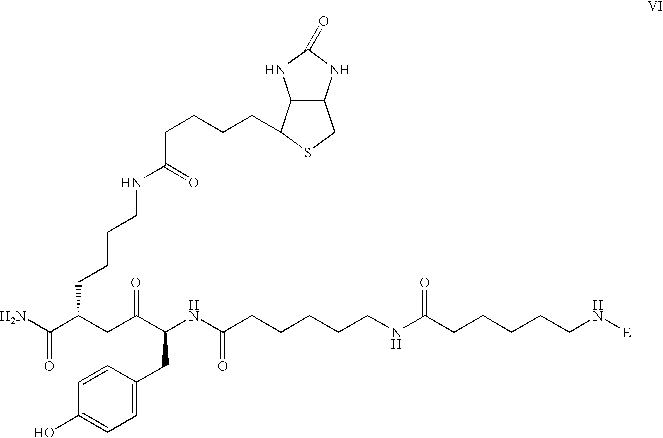

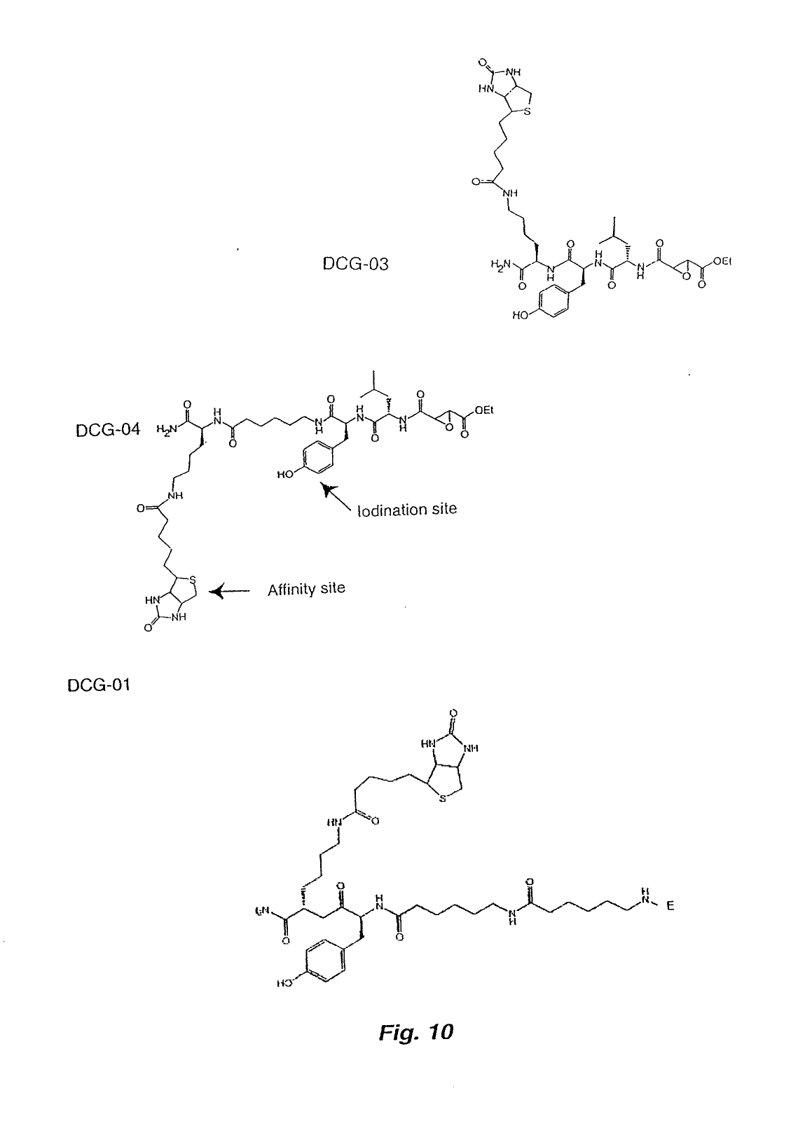

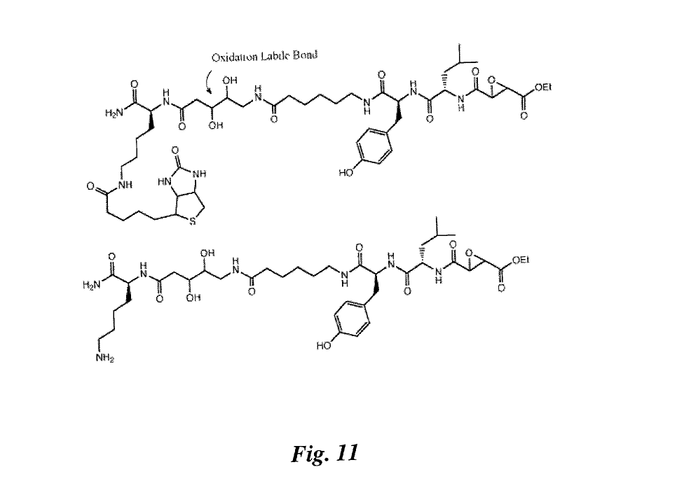

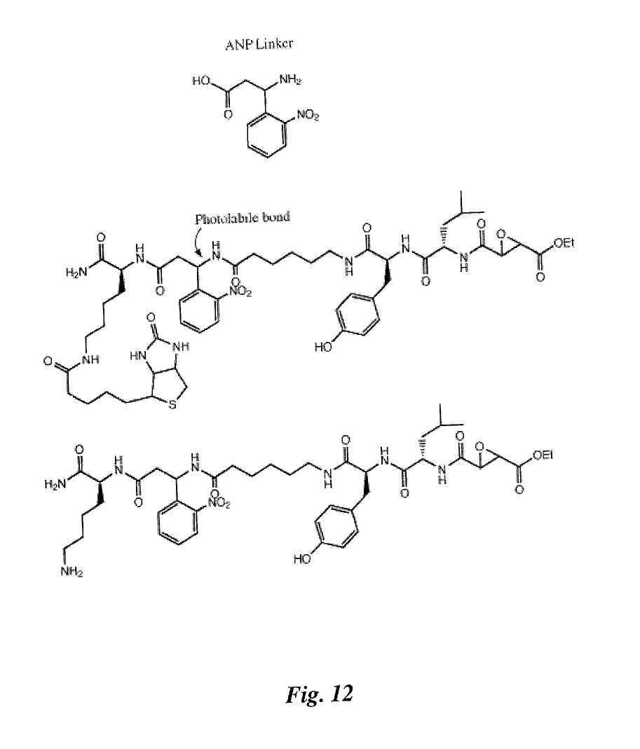

Particularly preferred probes of this invention are illustrated in FIG. 10.

The following formulae illustrate probes of this invention having an oligopeptide chain linked to a fluorescent moiety. These probes are illustrated by Formulas VII through X (designated BODIPY5581568-DCG-04, BODIPY493/503-DCG-04, BODIPY530/550-DCG-04, and BODIPY588/61 6-DCG-04, respectively).

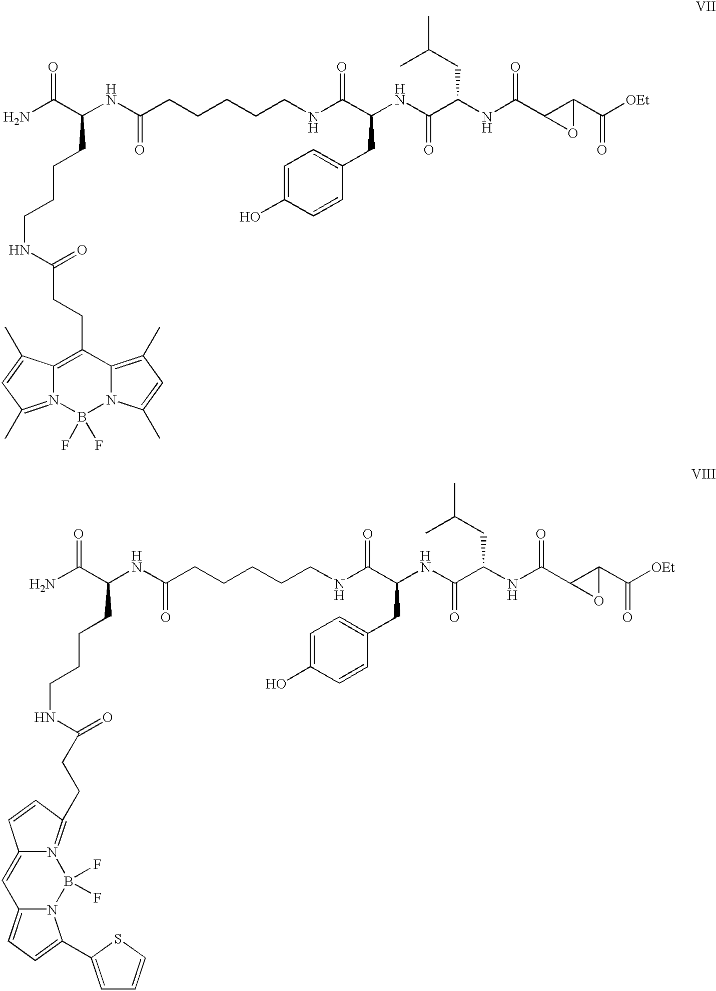

II. Probe Synthesis

Depending on the composition of the probe, various protocols may be used for synthesizing the probe. For the most part, the synthesis will involve the use of synthons or building blocks, particularly where the probe is an oligomer. For preparing oligomers, it will generally be useful to use a solid support and build the oligomer on the solid support in accordance with known methods. Where the probe has only three elements, the reactive group or electrophile, the hydrophobic group or binding specificity group, and the ligand, the synthesis may be performed in solution, where the order of combining the individual components may be varied.

The probes of this invention can be synthesized according to standard methods known to those of skill in the art. It is noted that methods of coupling electrophiles, and other molecules, to peptides are well known. However, in preferred embodiments, particularly where the electrophile is an epoxide, this invention provides an improved synthesis method. Briefly, this method involves a combination of solution and solid phase chemistries. The solution phase synthesis of the epoxide acid building block starting from commercially available diethyl tartrate is shown in FIG. 2A. Standard solid-phase peptide chemistry is used to build the peptide portion of the probe (e.g. DCG-04) and related compounds (see, e.g., FIG. 2B). This methodology provides a flexible system with which to incorporate virtually any peptide sequence prior to attachment of the electrophilic epoxide. It was a surprising discovery of this invention that the epoxy acid building block was stable to standard solid-phase peptide synthesis cleavage conditions (95% TFA).

The use of solid-phase chemistry also facilitates the synthesis of a diverse library in which, for example, the P2 leucine of DCG-04 is replaced with each of the natural amino acids (except cysteine due to reactivity with the epoxide and methionine due to oxidation).

By having the electrophile with a functionality, either amino or carboxy that can be joined to a solid support and a second functionality for linking to an amino acid, the subject compounds can be synthesized on a solid support by stepwise addition. In the subject compounds the electrophile is linked to the hydrophobic group by an amide bind with the electrophile supplying the carboxyl and the hydrophobic group providing the amino group. The amide group could be in the reverse direction. To some degree, the order of addition is arbitrary, except that where there are many functionalities on the units, one must devise a protocol for protection and deprotection that restricts bond formation to form the desired linkage. For the purposes of this invention, the direction of the linking functional groups may be in either direction where there is asymmetry, as with amides and esters.

Fluorophores are readily provided in place of the affinity tag, e.g. by synthesizing a free amine version of the probe (e.g. Formula XI) and reacting it with the succinamide ester of the fluorophore. Such derivatized fluorophores are readily obtained from Molecular Probes (Oregon, Calif.).

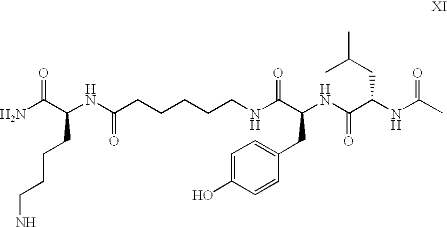

III. Probe Libraries

In another embodiment, this invention provides libraries of probes of this invention. Preferred libraries include, at least two, preferably at least five, more preferably at least ten, and most preferably at least twentynine different probes as described herein (e.g. of formula I-X),. In certain preferred probe libraries, each species of probe comprises a different and distinguishable label. Certain preferred probe libraries comprise a library of probes comprising amino acid or dipeptide protease binding/recognition domains. Certain preferred libraries comprise a dipeptide of the form: -Tyr-X- where X is essentially any other amino acid. In one particularly preferred library X includes, essentially any other naturally occurring amino acid and norleucine.

The libraries can be provided in any of a wide variety of formats. Thus, for example, all the members of a probe library can be combined into a single probe mixture. Alternatively, each probe can be provided in a separate container.

In embodiments, well suited for high throughput screening applications, the probe library is provided as one or more microtiter plates (e.g. 96 well plate, 384 well plate, etc.), with each library member (or several library members) in each well. Microtiter plate formats are well suited to handling and manipulation using laboratory robotic systems.

The probes of this invention can be also be provided attached to a substrate. Suitable substrates included, but are not limited to, solid surfaces, membranes, or gels. Substrate materials include, but are not limited to plastics, glass, quartz, metals, ceramics, and the like. In preferred embodiments, the probes (e.g. a probe library) is attached to a single contiguous surface or to a multiplicity of surfaces juxtaposed to each other (e.g. to a collection of beads or other particles).

When the probe library is attached to a surface it forms a probe array suitable for a wide variety of assays including, but not limited to, fingerprinting tissue cysteine protease activities, providing an activity profile of a cysteine hydrolase, and the like.

The probes of this invention can be coupled to a substrate according to any of a number of methods well known to those of skill in the art. Such methods include, but are not limited to, simple adsorption cross-linking with the use of linkers, or attachment by way of the affinity tag. In particularly preferred embodiments, the probes are attached to the surface by the affinity tag. Thus, for example, a surface bearing a streptavidin, or a modified streptavidin will bind a biotin affinity tag on the probe. Similarly, a surface bearing a Ni-NTA moiety will bind to a HiS₆ affinity tag. The selection of the affinity tag and the binding moiety will determine whether or not it is possible to subsequently release the probe(s) from the surface. Thus, for example a Ni-NTA-His₆ coupling is reversible. Similarly, while a biotin-streptavidin coupling is typically not cleavable, monomeric avidin has a reduced binding affinity biotin the bound probe can competitively eluted with high concentrations of biotin (e.g., 2 mM).

In certain embodiments, the probes of this invention are provided attached to beads and/or a polymeric resin that can be packed into a column. A sample (e.g. crude cell extract) can be run through the column where permitting probe targets to be bound. The bound target can then, optionally, be eluted.

IV. Assays Using Cysteine Hydrolase Probes

The cysteine hydrolase probes of this invention are useful in a wide variety of contexts. The probes may be specific for a range of different groupings of cysteine hydrolases, across one group of cysteine hydrolases, e.g. CA, CB and CD, or for one or a few cysteine hydrolases. Preferred uses include include, but are not limited to the analysis of cysteine hydrolase activities in crude cell extracts, identification of novel hydrolases, profiling of cysteine hydrolase activity in disease states, screening of candidate compounds for cysteine hydrolase activity, and tracking of cysteine hydrolases in biological fluids and tissue samples.

The probes of this invention can comprise ligands that include an affinity tag (e.g. biotin), ligands that facilitates detection of probe bound target molecules using, e.g., an affinity blot protocol (e.g. Western Blotting and labeling with a ligand specific to the affinity tag). In certain embodiments, a detectable label (e.g. a fluorescent label) is used instead of the affinity tag allowing rapid probe detection using, e.g., various fluorometric methods. The affinity tag or ligand also facilitates the temporary or permanent attachment of the probe(s) to a substrate whereby the probes(s) form effective affinity “chromatography” ligands facilitating isolation and characterization of the bound cysteine hydrolase(s).

For convenience, the ligand- or affinity tag-bearing probes of this invention can also be labeled with a detectable label (e.g. a radioactive label) thereby providing a bi-functional probe. The detectable label facilitates rapid detection of bound target molecules even in crude protein mixtures. For example, analysis of the labeling of DC2.4 lysates (see Example 1) by both affinity blot and label-detection (e.g. auto-radiography) techniques resulted in similar profiles of modified target molecules, highlighting the utility of both techniques. The presence of the affinity tag facilitated rapid isolation and further characterization of the tagged target molecule(s). Ultimately, the ability to use both autoradiography as well as blot techniques enhances the flexibility of the probes of this invention.

In various embodiments, as described below, the libraries of the probes of this invention can be used to identify a cysteine hydrolase and/or to provide a profile of a cysteine hydrolase's specificities (i.e. a hydrolase/protease fingerprint). The activity of individual cysteine hydrolases or a plurality of hydrolases can be profiled in a particular tissue or collection of tissues to characterize tissue-specific differences in cysteine hydrolase activities, to characterize developmental changes in cysteine hydrolase activities, to characterize changes in cysteine hydrolase activity in response to altered environmental conditions, to characterize changes in cysteine hydrolase activity associated with disease progression, to provide a fingerprint that is a measure of disease stage or progression, and the like.

A) Identifying and/or Isolating and/or Characterizing Cysteine Hydrolases

1) Protease Fingerprinting

In one embodiment, this invention provides methods of identifying an “activity profile” for a particular cysteine hydrolase or a group of cysteine hydrolases. IN preferred embodiments, such an activity profile comprises a measure of the activity of a plurality of probes of this invention for one or more cysteine hydrolases. Thus, in preferred embodiments, “fingerprinting” generally involves determining the binding of one or more probes of this invention, preferably of a library of probes of this invention to a particular cysteine hydrolase or group of cysteine hydrolases. This can be done using a “direct labeling” assay, however, preferably, a competitive assay is used (e.g. using a known inhibitor of the target protease). This assay generates an activity profile showing the relative binding of each probe comprising the library to the target protease.

The generation of such a profile for cathepsin H is illustrated herein in Example 1 (FIG. 6B). Pre-incubation of purified cathepsin H with the library of compounds followed by ¹²⁵I-DCG-04 labeling resulted in a specificity profile that was remarkably similar to the profile observed for cathepsin B in crude extracts (FIG. 6C). The data showed that although the two proteases are quite different in their biological functions, they have similar inhibitor specificity in the S2 pocket.

Since it is unlikely that two distinct proteases will exhibit identical reactivity across a diverse set of inhibitors, it is possible to use this information from positional scanning inhibitor libraries to generate “specificity fingerprints” for a series of well characterized hydrolases.

It is believed that creation of a database of cysteine hydrolase inhibitor profiles can be used to establish target identification by labeling of crude protein mixtures in the presence of compound libraries. The labeling pattern (fingerprint) is read and compared to the database. Identification of similar or the same fingerprint(s) in the database provides an indication of the cysteine hydrolase(s) present in the sample.

2) Identification and Characterization of Unknown Hydrolases

The methods of this invention can also be used to characterize known cysteine hydrolases and/or to identify, isolate, and/or characterize unknown cysteine hydrolases. These methods involve contacting a biological sample with one or more probes of this invention and detecting biding of the probe(s) with component(s) of the sample. Because the probes of this invention are generally specific to cysteine hydrolases, binding of the probes to a component in the sample initially indicates the presence of a cysteine hydrolase. Use of a library of probes of this invention increases the likelihood of detecting cysteine hydrolases, but requires no assumptions regarding which cysteine hydrolases are present in the sample.

Once specific binding is identified, the affinity tag on the probe(s) can readily be used to capture and isolate the bound cysteine hydrolase. The isolated hydrolase is then readily subjected to further analysis (e.g. tryptic digests, amino acid analysis, mass spectrometry, etc.). The isolation and subsequent analysis of isolated peptides is illustrated in Example 1.

In certain embodiments, the affinity tag is attached to the specificity determinant using a cleavable linker. Cleavable linkers circumvent problems of background proteins and endogenously biotinylated proteins or peptides that non-specifically stick to affinity resins during purification of protease targets from crude protein extracts. The cleavable linker is used to join the probe to a resin (e.g. Affigel from BioRad) to create an affinity resin. The “affinity resin” can be directly incubated with crude protein extracts and then be stringently washed (e.g., high SDS, low pH, boiling, etc.) to assure elimination of non-specific background proteins or peptides. The is followed by release of specific peptide products by trypsin digestion, acid hydrolysis, mild oxidation, or photorelease resulting in cleavage of the linker and release of the probe modified peptides. This method coupled with trypsin digestion and washing of the affinity resin prior to specific elution allows direct mass spectrometry of only active site peptides of modified cysteine proteases targets. The methods therefore can be used to obtain sequence information for multiple active site peptides simultaneously without the need for resolution by gel electrophoresis.

B) Profiling Protease Activity in a Tissue (Activity Monitoring)

One or more probes of this invention can be used to provide a cysteine hydrolase “activity profile” in one or more tissue types. Essentially any cell, tissue, or biological fluid can be subjected to such profiling as long as it contains one or more cysteine hydrolases. Selection of particular cells or tissues for such profiling allows cysteine hydrolase expression to be evaluated in a wide variety of contexts as indicated above. A few examples are illustrated below and in Example 1.

1) Profiling Disease Progression

One or a plurality of probes of this invention can be used to profile the progression of a disease. The subject probes preferably find application where a disease state results in the up or down regulation of the expression or activity of at least one of the enzymes to which the probes bind. While essentially any disease state can be profiled, disease progressions that are expected to involve cysteine hydrolase activity are particularly well suited for such profiling. One class of such diseases includes diseases characterized by tissue remodeling (e.g. various cancers, in particular invasive and/or metastatic cancers, rheumatoid arthritis, osteoporosis, and the like).

The use of the probes of this invention to profile progression of a cancer is illustrated in Example 1. While this Example uses a single, broadly reactive probe, the same methodologies can be used with a library of probes of this invention to provide a more detailed profile of the disease progression. Where the probes have different specificity the pattern of binding of each of the probes can be used to identify the individual enzymes or classes of enzymes and the level of activity for each of the enzymes or classes of enzymes.

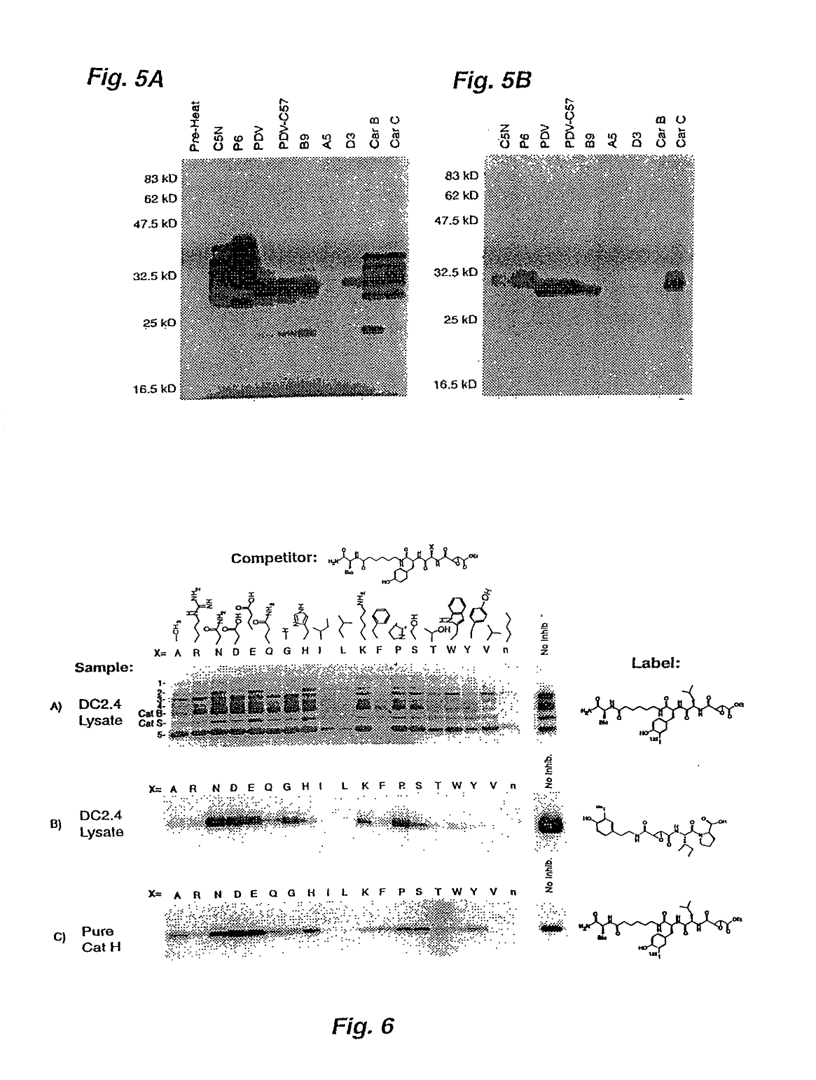

As described in Example 1, the mouse skin model of multi-stage carcinogenesis was profiled using a single probe of this invention (DCG-04). Ten cell lines representing various steps in the progression from benign skin cell (C5N) to highly invasive spindle cell carcinomas (CarB and CarC) were used to analyze global changes in activity of cathepsins throughout this multi-stage carcinogenesis model. The carcinoma progression also included benign papilloma cell lines P6, PDV and PDV-C57, and more invasive squamous cell carcinoma cell lines B9, A5, D3. Equal amounts of protein from each cell lysate were labeled with both the broadly reactive probe, ¹²⁵I-DCG-04, as well as the cathepsin B-specific probe, ¹²⁵I-MB-074 at pH 5.5 (FIG. 5). The results showed that several protease activities, including cathepsin B, dramatically fluctuate across the panel of cell lines.

The data provided in Example 1 illustrate that cells isolated from different tumor sources have different protease activity profiles. These profiles can be used, e.g., in a database to relate the profile to various aspects of the tumor cells, for example, the aggressiveness of the disease, the response to treatment, changes in tumor status, etc. Without being bound to a particular theory, it is believed that this signature of protease activity may in fact be unique to each cell and/or tumor much the same way genomics studies have shown that individual tumor cells have unique global gene expression profiles.

These methods can be used to profile the progression of essentially any disease state in which cysteine hydrolases play a role and their regulation and activity vary with the status of the disease, particularly as it relates to therapeutic treatment, advances and regressions or remissions. One may use an established model system or create an independent model system. Tissue biopsies, cells, and the like can be obtained, for example, from patients diagnosed at particular stages of a particular disease and characteristic profiles can be determined for the various disease stages.

Cysteine hydrolase expression/activity produced using particular probes of this invention in tissues obtained from characteristic stages of a disease progression can be entered into a database of such profiles. This database, or particular entries in such a database, can provide a reference or characteristic profile useful for staging or diagnosing or evaluating the prognosis of a disease. In such an embodiment, a sample is obtained from a subject and a cysteine hydrolase activity profile is determined using one or more particular probes of this invention. The resulting activity profile is then compared to a one or more “reference” profiles, e.g. stored in a database. If the measured profile is sufficiently similar to, or “identical” with, a reference profile and that reference profile is characteristic of a particular disease, particular disease stage, or prognosis, it can be inferred that the subject exhibits that disease, disease stage, or prognosis. Such a determination, need not be definitive of such a disease, disease stage, or prognosis, but can simply serve as a component of a differential diagnosis which can utilize known disease indicators. The determination of disease state or prognosis can then inform decisions regarding a treatment regimen (e.g. the decision whether or not to use chemotherapy and/or radiotherapy in addition to surgery in the treatment of a cancer patient, etc.).

2) Profiling Across Tissue Types

One or more probes of this invention can be used to profile cysteine hydrolase expression in a variety of tissue types. Thus, hydrolase activity in diseased tissues can be compared to healthy tissues, hydrolase activity in differentiated cells can be compared to undifferentiated cells, changes in cysteine hydrolase activity in response to environmental conditions or drugs, and the like can be assayed. Such profiles can be determined using a single probe, however in most cases, a probe library is used.

The creation of such a tissue profile using a probe library is illustrated in Example 1. In this Example, a small library of compounds is employed in which the peptide recognition portion of the molecule was varied. A complete scanning library consisting of 18 natural amino acids and the isosteric methionine analog norleucine was constructed substituting the various amino acids for leucine in DCG-04. This library of inhibitors was used to create profiles of inhibitor specificity for proteases targeted by DCG-04 and MB-074 (FIG. 6).

Competition analysis was used to determine the potency of each member of the P2 scanning library towards multiple protease targets. Lysates from DC2.4 cells were pre-incubated with 50 μM of each of the 19 DCG library members and residual activity measured for multiple proteases using ¹²⁵I-DCG-04 (FIG. 6A). In general, residues containing non-charged aliphatic side chains, isoleucine (I), leucine (L; DCG-04) and norleucine (n), showed highest activity and the lowest amount of specificity across the profile of polypeptides . More interesting was the apparent selectivity of several DCG family compounds for a subset of labeled polypeptides. For example, the valine containing compound competed for polypeptides 1, 2 and cathepsin B but had little effect on the remaining compounds. In contrast, both the phenylalanine and tyrosine containing compounds showed specificity for polypeptides 2, 3, 4, and 5. Furthermore, while the aspartic acid and glycine containing compounds showed relatively poor activity overall, they showed some degree of specificity against polypeptide 2. Similar methods can be used to profile essentially any tissue.

C) Screening for Modulators of Cysteine Hydrolase Activity

The methods described herein for profiling tissue types or for profiling individual hydrolases can also be used to screen for modulators of cysteine hydrolase activity. Basically, the biological sample is contacted with one or more test agents. The sample is then profiled for cysteine hydrolase activity with one or more probes of this invention as described above. In addition, different concentrations of modulators can be used to establish the dose response of modulation of cysteine protease activity. This method can also be used to determine the selectivity of a given modulator with respect to all cysteine protease targets of one or more probes of this invention.

The sample (e.g. crude cell extract) can be contacted with the agent directly. Alternatively, where the sample is a cell line, the cell line can be contacted with the test agent, or cultured in the presence of the test agent. In other embodiments, the test agent can be administered to an animal and biological samples derived from the animal are profiled as described above.

When the sample contacted with the test agent shows a cysteine hydrolase activity profile different from the profile obtained from a negative control (e.g. a sample contacted with a lower amount of test agent or no test agent) it is inferred that the test agent modulates cysteine hydrolase activity. The assays of this invention are typically scored as positive where there is a difference between the activity seen with the test agent present and the (usually negative) control, preferably where the difference is statistically significant (e.g. at greater than 80%, preferably greater than about 90%, more preferably greater than about 98%, and most preferably greater than about 99% confidence level). Most preferred “positive” assays show at least a 1.2 fold, preferably at least a 1.5 fold, more preferably at least a 2 fold, and most preferably at least a 4 fold or even a 10-fold difference from the negative control.

1) Agents for Screening: Combinatorial Libraries (e.,g., Small Organic Molecules)

Virtually any agent can be screened according to the methods of this invention. The term “test agent” refers to any agent that is to be screened for a desired activity. The “test composition” can be any molecule or mixture of molecules, optionally in a suitable carrier. Such agents include, but are not limited to nucleic acids, proteins, sugars, polysaccharides, glycoproteins, lipids, and small organic molecules, both naturally occurring and synthetic. Preferred test agents include small organic molecules.

Conventionally, new chemical entities with useful properties are generated by identifying a chemical compound (called a “lead compound”) with some desirable property or activity, creating variants of the lead compound, and evaluating the property and activity of those variant compounds. The current trend is to shorten the time scale for all aspects of drug discovery. Because of the ability to test large numbers quickly and efficiently, high throughput screening (HTS) methods are replacing conventional lead compound identification methods.

In one preferred embodiment, high throughput screening methods involve providing a library containing a large number of potential therapeutic compounds (candidate compounds). Such “combinatorial chemical libraries” are then screened in one or more assays, as described herein to identify those library members (particular chemical species or subclasses) that display a desired characteristic activity (e.g. ability to modulate a cysteine protease activity, or activity profile). The compounds thus identified can serve as conventional “lead compounds” or can themselves be used as potential or actual therapeutics.

A combinatorial chemical library is a collection of diverse chemical compounds generated by either chemical synthesis or biological synthesis by combining a number of chemical “building blocks” such as reagents. For example, a linear combinatorial chemical library such as a polypeptide (e.g., mutein) library is formed by combining a set of amino acids in multiple different orders for a given number of amino acid units. Millions of chemical compounds can be synthesized through such combinatorial mixing of chemical building blocks. For example, one commentator has observed that the systematic, combinatorial mixing of 100 interchangeable chemical building blocks results in the theoretical synthesis of 100 million tetrameric compounds or 10 billion pentameric compounds (Gallop et al. (1994) 37(9): 1233-1250).

Preparation of combinatorial chemical libraries is well known to those of skill in the art. Such combinatorial chemical libraries include, but are not limited to, peptide libraries (see, e.g., U.S. Pat. No. 5,010,175, Furka (1991) Int. J. Pept. Prot. Res., 37: 487-493, Houghton et al. (1991) Nature, 354: 84-88). Peptide synthesis is by no means the only approach envisioned and intended for use with the present invention. Other chemistries for generating chemical diversity libraries can also be used. Such chemistries include, but are not limited to: peptoids (PCT Publication No WO 91/19735, Dec. 26, 1991), encoded peptides (PCT Publication WO 93/20242, Oct. 14, 1993), random bio-oligomers (PCT Publication WO 92/00091,Jan. 9, 1992), benzodiazepines (U.S. Pat. No. 5,288,514), diversomers such as hydantoins, benzodiazepines and dipeptides (Hobbs et al., (1993) Proc. Nat. Acad. Sci. USA 90: 6909-6913), vinylogous polypeptides (Hagihara et al. (1992) J. Amer. Chem. Soc. 114: 6568), nonpeptidal peptidomimetics with a Beta-D-Glucose scaffolding (Hirschmann et al., (1992) J. Amer. Chem. Soc. 114: 9217-9218), analogous organic syntheses of small compound libraries (Chen et al. (1994) J. Amer. Chem. Soc. 116: 2661), oligocarbamates (Cho, et al., (1993) Science 261:1303), and/or peptidyl phosphonates (Campbell et al., (1994) J. Org. Chem. 59: 658). See, generally, Gordon et al., (1994) J. Med. Chem. 37:1385, nucleic acid libraries (see, e.g., Strategene, Corp.), peptide nucleic acid libraries (see, e.g., U.S. Pat. No. 5,539,083) antibody libraries (see, e.g., Vaughn et al. (1996) Nature Biotechnology, 14(3): 309-314), and PCT/US96/10287), carbohydrate libraries (see, e.g., Liang et al. (1996) Science, 274: 1520-1522, and U.S. Pat. No. 5,593,853), and small organic molecule libraries (see, e.g., benzodiazepines, Baum (1993) C&EN, January 18, page 33, isoprenoids U.S. Pat. No. 5,569,588, thiazolidinones and metathiazanones U.S. Pat. No. 5,549,974, pyrrolidines U.S. Pat. Nos. 5,525,735 and 5,519,134, morpholino compounds U.S. Pat. No. 5,506,337, benzodiazepines U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.).

A number of well known robotic systems have also been developed for solution phase chemistries. These systems include, but are not limited to, automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.) which mimic the manual synthetic operations performed by a chemist and the Venture™ platform, an ultra-high-throughput synthesizer that can run between 576 and 9,600 simultaneous reactions from start to finish (see Advanced ChemTech, Inc. Louisville, Ky.)). Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

2) High Throughput Screening

Any of the assays for compounds modulating the activity of cysteine hydrolases described herein are amenable to high throughput screening. The biological samples utilized in the methods of this invention need not be contacted with a single test agent at a time. To the contrary, to facilitate high-throughput screening, a single sample may be contacted by at least two, preferably by at least 5, more preferably by at least 10, and most preferably by at least 20 test compounds. If the sample scores positive, it can be deconvoluted, e.g., subsequently tested with a subset of the test agents until the agents having the activity are identified.

Robotic high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols the various high throughput. Thus, for example, Zymark Corp. provides technical bulletins describing screening systems for detecting the modulation of gene transcription, ligand binding, and the like.

D) Assay Formats

Preferred probes of this invention can form an essentially irreversible bond with their target cysteine hydrolases. Because the target molecule/probe complex is so stable, it can be subjected to a wide variety of chemical procedures including, but not limited to, a wide variety of methods used for protein purification and analysis (e.g., gel electrophoresis, anion exchange chromatography reverse phase high performance liquid chromatography (HPLC), capillary electrophoresis, entropic trap electrophoresis, etc.). In particularly preferred embodiments, the labeled probe/target complex is analyzed using gel electrophoresis and/or Western blotting methods, e.g. as described in Example 1.

In certain embodiments, the assays of this invention involve direct labeling of the target cysteine hydrolase (e.g. with a radiolabeled probe of this invention) or indirect labeling, (e.g. a competition assay). In a direct labeling assay, the labeled probe is contacted with the biological sample under conditions where the probe can specifically bind to its target cysteine hydrolase(s) if they are present. Typically, the labeled cysteine hydrolase(s) will be separated, e.g. using SDS-PAGE, 2-D electrophoresis, etc., and the label is detected (e.g. using autoradiography for a radioactive label) to provide an indication of the presence and/or amount of labeled target.

In an indirect assay, such as competitive assay, the probe(s) of this invention are contacted with the biological sample during or after contacting of the sample with a reagent that specifically binds to that sample. The reagent can be a probe of this invention or a different type of probe. Probe binding to the target is a function of the relative affinity of the probe to the target as compared to the competing reagent. Either the probe or the competing reagent can be labeled and assayed. A detailed protocol for a competitive binding assay is provided in Example 1.

In certain embodiments, the probe(s) of this invention are immobilized on a solid support. The sample is labeled (e.g. with a radioactive label) and contacted to the probes which then act as an affinity matrix specifically binding the target cysteine hydrolase(s). Detection of the bound labeled sample components provides an indication of binding.

In such an embodiment, the probe rather than the sample can be labeled. By employing cleavable linkers and releasing the probes from the solid support, the sample components that are not bound by the probe will show different mobility in an electrophoretic gel and are easily distinguished from the target-bound probes. Solid-phase assays of this sort are particularly well suited for high-throughput screening systems. It is also contemplated that such solid-phase assays can be scaled down to “chip-based” formats for rapid screening. Various “lab on a chip” formats are well known to those of skill in the art (see, e.g., U.S. Pat. Nos. 6,132,685, 6,123,798, 6,107,044, 6,100,541, 6,090,251, 6,086,825, 6,086,740, 6,074,725, 6,071,478, 6,068,752, 6,048,498, 6,046,056, 6,042,710, and 6,042,709) and may readily be adapted to the assays of this invention.

Assays of this invention are also amenable to solution phase chemistries. In one such embodiment, the biological sample and the probe(s) of this invention are labeled with different detectable labels. When the probe(s) bind a target, the target and probe labels co-localize. Detection of the co-localized labels provides a measurement of bound cysteine hydrolase. The co-localized labeled entities can be isolated and captured using the affinity tag on the probe and then subjected to subsequent analysis.

F) Assay Controls

The assays of this invention preferably include a control for non-specific binding. One particularly preferred control comprises a biological sample in which the proteins are denatured (e.g. by heating). Apparent signals generated in such a control are discounted (e.g. subtracted) from the signals read in the test assay. After such a “substraction” the remaining signal is presumably due to specific binding of the probe(s) to their target proteins.

G) Preferred Biological Samples

The biological samples, used herein, include, but are not limited to samples obtained from an organism, from components (e.g., cells or tissues) of an organism, and/or from in vitro cell or tissue cultures. The sample can be of any biological tissue or fluid (e.g. blood, serum, lymph, cerebrospinal fluid, urine, sputum, etc.). Biological samples can also include organs or sections of tissues such as frozen sections taken for histological purposes. In certain embodiments, the biological samples include crude cell extracts. The extracts can include essentially unpurified cell lysates. The cell lysates can be treated (e.g. centrifuged) to remove particulate matter. Alternatively, the crude cell extracts can comprise isolated cellular “total” protein.

The biological samples can be derived from any organism that comprises a cysteine hydrolase. Such organisms include, but are not limited to various prokaryotes and essentially all eukaryotic organisms. Preferred organisms include bacteria, fungi, plants, invertebrates and vertebrates. Particularly preferred organisms include mammals (e.g. a rodent, largomorph, murine, bovine, canine, equine, non-human primate, human, etc.).

V. Databases of Cysteine Protease Activity Profiles

In certain embodiments, the methods of this invention further comprise listing the identified cysteine hydrolases and their activity profiles (as determined by a particular set of probes) in a database identifying activity profiles for various proteins. Similarly, activity profiles for various tissues can also be entered into databases associating tissues with activity profiles for particular activity probes or sets of activity probes.

The data structures produced by the methods of this invention, or the members of such data structures (i.e., the activity profiles) can be used as reference objects in database searches. Thus, it is possible to use the database to store, retrieve, search and identify similar or identical activity profiles. Comparison of a profile obtained in an assay with a database of profiles may provide an indication as to the cysteine hydrolase composition of the sample, and/or of the physiological state or healthy of the organism from which the sample is derived.

The term “database”, as used herein, refers to a means for recording and retrieving information. In preferred embodiments the database also provides means for sorting and/or searching the stored information. The database can comprise any convenient media including, but not limited to, paper systems, card systems, mechanical systems, electronic systems, optical systems, magnetic systems or combinations thereof. Preferred databases include electronic (e.g. computer-based) databases. Computer systems for use in storage and manipulation of databases are well known to those of skill in the art and include, but are not limited to “personal computer systems”, mainframe systems, distributed nodes on an inter- or intra-net, data or databases stored in specialized hardware (e.g. in microchips), and the like.

VI. Probe Kits

This invention also provides kits for practice of the methods described herein. In certain embodiments the kits comprise a container containing one or more of the probes of this invention. In particularly preferred embodiments the kits comprise a plurality of probes of this invention (e.g. a probe library). In certain embodiments, the probe(s) are provided attached to a solid support. The kits can, optionally, further include one or more known inhibitors (e.g. suicide substrate) of a cysteine hydrolase.

The kits may optionally include any reagents and/or apparatus to facilitate practice of the methods described herein. Such reagents and apparatus include, but are not limited to buffers, instrumentation, microtiter plates, labeling reagents streptavidin or biotin conjugated substrates, PAGE gels, blotting membranes, reagents for detecting a signal, and the like.

In addition, the kits can include instructional materials containing directions (i.e., protocols) for the practice of the methods of this invention. Preferred instructional materials provide protocols for utilizing the kit contents for screening for cysteine hydrolase activity and/or for activity fingerprinting as described herein. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The following examples are offered to illustrate, but not to limit the claimed invention.

### Example 1

### Epoxide Electrophiles as Activity-dependent Cysteine Protease Profiling and Discovery Tools

This example illustrates the design and use of chemical probes that can be used to broadly track activity of cysteine proteases. The structure of the general cysteine protease inhibitor E-64 was used as a scaffold. Analogs were synthesized by varying the core peptide recognition portion while adding affinity tags (biotin and radio-iodine) at distal sites. The resulting probes containing a P2 leucine residue (DCG-03 and DCG-04) targeted the same broad set of cysteine proteases as E-64 and were used to profile these proteases during the progression of a normal skin cell to a carcinoma. A library of DCG-04 derivatives was constructed in which the leucine residue was replaced with all natural amino acids. This library was used to obtain inhibitor activity profiles for multiple protease targets in crude cellular extracts. Finally, the affinity tag of DCG-04 allowed purification of modified proteases and identification by mass spectrometry.

This example thus illustrates a simple and flexible method for functionally identifying cysteine hydrolases while simultaneously tracking their relative activity levels in crude protein mixtures. The probes described herein were used to determine relative activities of multiple proteases throughout a defined model system for cancer progression. Information obtained from libraries of affinity probes provided a rapid method for obtaining detailed functional information without the need for prior purification/identification of targets.

### Results and Discussion

### Design and Synthesis of DCG-04

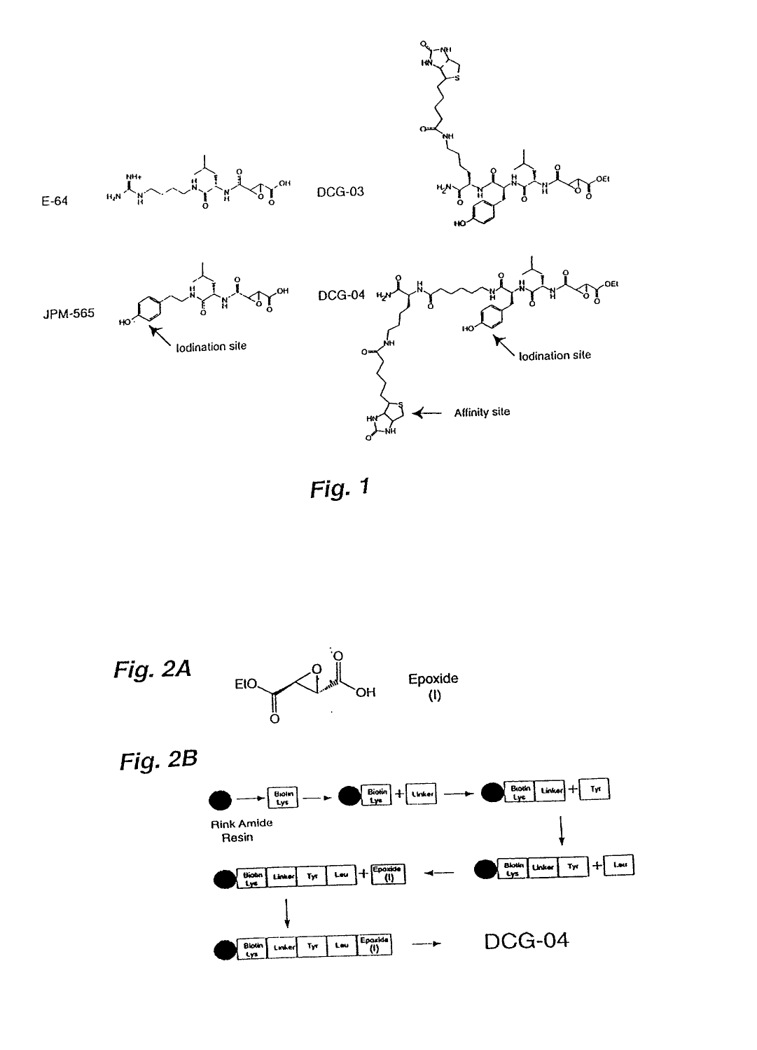

The natural product E-64 is a promiscuous irreversible cysteine protease inhibitor that is broadly reactive toward the papaine family of cysteine proteases (Barrett and Hanada (1982) Biochem. J., 201: 189-198) (FIG. 1). Its leucine sidechain mimics the P2 amino acid of a substrate, occupying the target's S2 binding pocket while the agmatine moiety binds in the S3 position (Matsumoto et al. (1999) Biopolymers 51: 99-107). Rich et al. synthesized JPM-565 (FIG. 1), a derivative in which a tyramine moiety replaces the agmatine side chain of E-64 (Meara and Rich (1996) J. Med. Chem. 39, 3357-3366; Shi et al. (1992) J. Biol. Chem. 267, 7258-7262). This closely related compound was found to have similar class-specific reactivity for cysteine proteases as E-64. Since the P2 position of a substrate is considered to be the main specificity determinant for many cysteine proteases, we reasoned that further extension of the non-prime binding portion of JPM-565 would not significantly perturb binding affinity for a target protease. In addition, modification to the non-prime side binding element of the E-64 derivative CA-074 had little effect on binding to cathepsin B (Bogyo et al. (2000) Chem Biol., 7: 27-38; 26.Bogyo et al. (1997) Proc. Natl. Acad. Sci., USA, 94: 6629-6634). Elaboration of the peptide portion of E-64 allowed both incorporation of an affinity tag as well as attachment of the compound to a solid support. The resulting bi-functional compounds, DCG-03 and DCG-04, contain both the iodinatable phenol ring of JPM-565 and the additional affinity site created by incorporation of a sidechain biotinylated lysine residue (FIG. 1). Addition (DCG-04) or removal (DCG-03) of an amino hexanoic acid spacer between the affinity site and the electrophile was used to determine the space requirement for binding and recognition of the affinity label by support-bound avidin.

Peptide epoxides were synthesized using a combination of solution and solid phase chemistries. The solution phase synthesis of the epoxide acid building block starting from commercially available diethyl tartrate is shown in FIG. 2A. Standard solid-phase peptide chemistry was used to build the peptide portion of DCG-04 and related compounds (FIG. 2B). This methodology provides a flexible system with which to incorporate virtually any peptide sequence prior to attachment of the electrophilic epoxide. Surprisingly, the epoxy acid building block was stable to standard solid-phase peptide synthesis cleavage conditions (95% TFA). The use of solid-phase chemistry also allowed the synthesis of a diverse library in which the P2 leucine of DCG-04 was replaced with each of the natural amino acids (except cysteine due to reactivity with the epoxide and methionine due to oxidation). The non-natural amino acid norleucine was used as an isosteric methionine analog. The results obtained using this 19 member library of compounds are described below.

### DCG-04 is an Activity-dependent Affinity Label

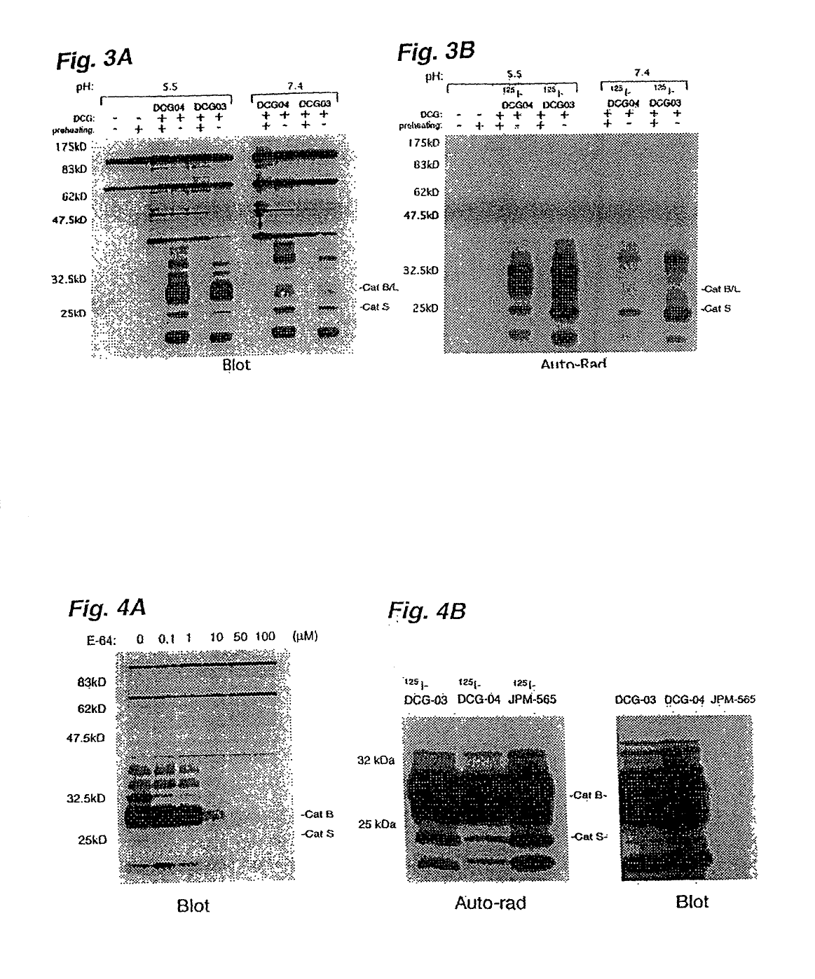

Dendritic cells express relatively high levels of lysosomal cathepsins, making them a logical source of material for establishing parameters for the use of DCG-04. FIG. 3 shows the labeling profile of polypeptides modified by incubation with either DCG-03, DCG-04, ¹²⁵I-DCG-03, or ¹²⁵I-DCG-04 followed by SDS-PAGE analysis. Radio-iodinated (autoradiogram) and non-radio-iodinated (blot) DCG-03 and DCG-04 labeled multiple polypeptides in the range of 20-40 kDa.

Although the intrinsic reactivity of the epoxide electrophile portion of DCG-04 towards free thiols is quite poor, we wanted to determine if DCG-04 and its derivatives were capable of non-specific alkylation of proteins in crude cellular extracts. A preheating control was used to reveal non-specific labeling, with the assumption that denatured, inactive proteins modified by DCG-03 and DCG-04 represent nonspecific modifications. Enzymatically active proteins were deduced by subtraction (FIG. 3). Labeling of all of the major species in the 20-40 kDa size range was lost upon heat denaturation of samples prior to addition of compounds suggesting that labeling is dependent on enzymatic activity and that these bands correspond to the major proteases in the extract. Several higher molecular weight species were observed by affinity blotting of both denaturing controls and samples in which no inhibitor was added. These species are likely to represent non-specific alkylations and endogenously biotinylated proteins.

Comparison of labeling, at neutral (pH 7.4) and at the acidic pH of the lysosome (pH 5.5), indicated that several of the modified polypeptides in the 30 kDa size range required reduced pH for activity. This result is consistent with reported findings that several lysosomal cysteine proteases either reversibly or irreversibly lose activity upon de-acidification of lysosomal compartments (Barrett et al. (1998) Handbook of Proteolytic Enzymes, Academic Press, San Diego).

Analysis of the labeling of DC2.4 lysates by both affinity blot and auto-radiography techniques resulted in similar profiles of modified polypeptides, highlighting the utility of both techniques. However, the auto-radiogram showed exclusive modification of enzymatically active polypeptides by radiolabeled forms of DCG-03 and DCG-04. Addition of the rather bulky iodine atom to DCG-03 and DCG-04 had only a modest effect on target modification yet resulted in compounds with dramatically reduced background labeling and increased sensitivity. Ultimately, the ability to use both autoradiography as well as blot techniques enhances the flexibility of these protease detection reagents and further highlights the utility of bi-functional inhibitors.

### DCG-04 Targets Cysteine Proteases Inhibited by E-64 and JPM-565

Both direct labeling and indirect competition experiments were performed to confirm that DCG-04 reacts with a similar subset of proteases to the parent compounds E-64 and JPM-565. An indirect competition experiment was required to determine the polypeptides modified by E-64 since it lacks an affinity label. Extracts from the dendritic cell line DC2.4 were preincubated with increasing concentration of E-64 followed by labeling with ¹²⁵I-DCG-04. Final labeling intensity was used to indirectly monitor extent of polypeptide modification by E-64. The competition revealed that all polypeptides labeled by ¹²⁵I-DCG-04 are effectively competed by E-64 indicating that the two compounds target the same subset of proteases (FIG. 4A). A similar competition experiment was performed using the cathepsin B specific inhibitor MB-074 (Bogyo et al. (2000) Chem Biol., 7: 27-38). These results positively identified the diffuse 30 kDa polypeptide (labeled cat B in FIG. 4A) as cathepsin B (data not shown).

Comparison of the specificity of DCG-03, DCG-04 and JPM-565 was accomplished using direct labeling of DC2.4 cell lysates. Labeling profiles obtained for ¹²⁵I-DCG-03, ¹²⁵I-DCG-04 and ¹⁵⁵I-JPM-565 were identical for all three probes and indicated that each targeted polypeptides in the 20-40 kDa size range (FIG. 4B). Analysis of non-radiolabeled DCG-03, DCG-04 and JPM-565 treated extracts again showed the similarity of the blotting and autoradiography detection systems. As expected, JPM-565, which lacks a biotin label, showed no labeling as detected by affinity blotting. Together these results establish that modifications to the extended binding portion of the E-64 family of compounds have little effect on selectivity or potency. However, this region of the inhibitor may still play an important role in establishing specificity of binding when equipped with the proper recognition sequence. Future work is aimed at exploring the use of extended peptide recognition motifs to fine tune selectivity of the DCG family of inhibitors for specific protease targets.

### Profiling Applications

The aforementioned methods established the initial parameters for use of the general cysteine protease labels DCG-03 and DCG-04. We next wanted to apply these techniques to profile the activity and specificity of cysteine proteases in several different model systems. The broadly reactive probe DCG-04 was used to generate activity profiles of multiple protease targets both in a model for disease progression and throughout multiple tissue types. Similarly, activity profiles were generated using the cathepsin B specific probe MB-074 to provide complementary information for a single, well-defined cysteine protease target. This information was also used to positively establish the identity of cathepsin B in the DCG-04 labeling profiles. To obtain more detailed functional information for DCG-04 modified proteases, inhibitor specificity profiles were generated using a library of DCG-04 analogs in total cellular extracts. The same libraries were also used in conjunction with the cathepsin B-specific probe, ¹²⁵I-MB-074, as well as with purified cathepsin H to determined specificity profiles for individual target proteases. These results are described below.

### Profiling Across Disease Progression Using DCG-04 and MB-074

The mouse skin model of multi-stage carcinogenesis has been well-studied genotypically and phenotypically, has discrete steps in the progression, but lacks information on cysteine protease involvement (Kemp et al. (1994) Cold Spring Harbor Symp. Quant. Biol. 59: 427-434; Yuspa et al. (1994) J. Investigative Dermatol., 103: 90S-95S). The role of cathepsins in tumor biology has mostly focused on cathepsin B and L. Up-regulated levels of both cathepsin B and L have been shown to correlate with an invasive phenotype (Yan et al. (1998) Biol. Chem., 379: 113-123; Baricos et al. (1988) Biochem. J. 252: 301-304). Furthermore, cathepsins B and L are secreted by many types of tumorigenic cells and treatment of invasive cells with the cysteine protease inhibitor E-64 results in a block in cellular invasion into a synthetic matrix (Linebaugh et al. (1999) Europ. J. Biochem., 264: 100-109; Mason et al. (1987) Biochem. J. 248: 449-454). These data indicate that cathepsins are likely to play an important role in the metastatic process.

Ten cell lines representing various steps in the progression from benign skin cell (C5N) to highly invasive spindle cell carcinomas (CarB and CarC) were used to analyze global changes in activity of cathepsins throughout this multi-stage carcinogenesis model. The carcinoma progression also includes benign papilloma cell lines P6, PDV and PDV-C57, and more invasive squamous cell carcinoma cell lines B9, A5, D3. Equal amounts of protein from each cell lysate were labeled with both the broadly reactive probe, ¹²⁵I-DCG-04, as well as the cathepsin B-specific probe, ¹²⁵I-MB-074 at pH 5.5 (FIG. 5). The results show that several protease activities, including cathepsin B, dramatically fluctuate across the panel of cell lines.

The broadly reactive probe ¹²⁵I-DCG-04 highlights the activity of several proteases in the lysosomal cysteine protease size range in each of the cell types (FIG. 5A). The benign cell lines C5N and P6 both contain multiple labeled polypeptides between 28 and 45 kDa, however, the labeling intensity observed for the P6 line is dramatically increased for all polypeptides in this range. Interestingly, the major difference between these cell lines is an activating mutation in the ras gene (Quintanilla et al. (1991 Carcinogenesis 12: 1875-1881). It has been previously shown that various classes of proteases, including the cathepsins, are upregulated downstream of Ras; however, these studies were limited to analysis of expression levels of cathepsin B and H (Kim et al. (1998) International J. Cancer 79: 324-333).

The papilloma cell lines PDV, and PDV-C57 show nearly identical patterns of labeling (FIG. 5A). However, these profiles are dramatically different than the profile observed for C5N and P6 lysates. A predominant 30 kDa polypeptide (cathepsin B; see below) is modified along with a less intensely labeled 21 kDa polypeptide. The squamous cell carcinoma cell lines B9, A5 and D3 result in a similar profile of polypeptides modified. While all three lines are nearly identical cancer cells types, only B9 shows appreciable labeling of the major 30 kDa and 21 kDa polypeptides. Similarly, the two highly invasive spindle cell carcinomas Car B and Car C show similar, but not identical, labeling profiles. The 21 kDa species, in particular, shows differential labeling in the two cell types. These findings illustrate that cells isolated from different tumor sources have different protease activities. This signature of protease activity may in fact be unique to each cell and/or tumor much the same way genomics studies by Browne and colleagues have shown that individual tumor cells have unique global gene expression profiles (Alizadeh and Staudt, (2000) [see comments]. Nature 403: 503-511).

The cathepsin B-specific label ¹²⁵I-MB-074 was used to directly examine the profile of cathepsin B activity in the same collection of cells described above (FIG. 5B). This probe has been found to label cathepsin B in a highly specific manner (Bogyo et al. (2000) Chem Biol., 7: 27-38). Labeling of cathepsin B dramatically changed across the profile of cell types with the greatest activity observed for the PDV and PDV-C57 lines. Furthermore, the apparent molecular weight as well as the sharpness of the cathepsin B band differed for the benign and spindle cell carcinomas suggesting that this enzyme is modified differently in these cell types. This change in migration for cathepsin B may be due to changes in glycosylation or other post translational modifications. Cathepsin B has been found to exist as different isoforms of differing pIs in various tumor cells as a result of changes in glycosylation and trafficking (Moin et al. (1998) Biol. Chem., 379: 1093-1099). Changes in the post-translational modification of cathepsin B is likely to effect the localization of active forms of the enzyme and therefore may play an important role in the control of cathepsin B activity in tumors (Moin et al. (1998) Biol. Chem., 379: 1093-1099). Overall, the results obtained from labeling with ¹²⁵I-MB-074 further highlight the variability of cathepsin B activity found in different types of tumor cells as well as in nearly identical cell lines derived from different sources.

### Profiling Protease Specificity Using a Library of Inhibitors

To take advantage of the flexibility and ease of synthesis of the DCG-04 family of compounds we created a small library of compounds in which the peptide recognition portion of the molecule is modified. It has been proposed that the main specificity regions within the active binding site of the cathepsins are S2, S1, S 1′, and S2′, with S2 containing the main binding pocket (Turk et al. (1998) Biol. Chem., 379: 137-147). Since the leucine residue of E-64 binds in the critical S2 pocket of many proteases (Matsumoto et al. (1999) Biopolymers 51: 99-107), changes to this residue are likely to have the greatest effect on specificity of our inhibitor for a given target. A complete scanning library consisting of 18 natural amino acids and the isosteric methionine analog norleucine was constructed. This library of inhibitors was used to create profiles of inhibitor specificity for proteases targeted by DCG-04 and MB-074 (FIG. 6).

Competition analysis was used to determine the potency of each member of the P2 scanning library towards multiple protease targets. Lysates from DC2.4 cells were pre-incubated with 50 μM of each of the 19 DCG library members and residual activity measured for multiple proteases using ¹²⁵I-DCG-04 (FIG. 6A). In general, residues containing non-charged aliphatic side chains, isoleucine (I), leucine (L; DCG-04) and norleucine (n), show highest activity and the lowest amount of specificity across the profile of polypeptides. More interesting was the apparent selectivity of several DCG family compounds for a subset of labeled polypeptides. For example, the valine containing compound competed for polypeptides 1, 2 and cathepsin B but had little effect on the remaining species. In contrast, both the phenylalanine and tyrosine containing compounds showed specificity for polypeptides 2, 3, 4, and 5. Furthermore, while the aspartic acid and glycine containing compounds showed relatively poor activity overall, they showed some degree of specificity against polypeptide 2. Using this data to simultaneously score inhibitors for potency and selectivity will be valuable for the development of specific inhibitors.

Similar competition experiments were performed with the library of DCG analogs to obtain profiles of single proteases. DC2.4 lysates were preincubated with P2 library and then labeled with the cathepsin B-specific compound ¹²⁵I-MB-074 (FIG. 6B). This method allowed analysis of cathepsin B specificity in crude extracts. As found in the ¹²⁵I-DCG-04 labeling (FIG. 6B), isoleucine, leucine, valine and norleucine analogs showed the highest activity followed by the aromatic amino acids (W, Y, F) containing compounds. In order to explore specificity profiles for additional cysteine proteases that could not be specifically labeled in crude extracts, we performed the same competition labeling experiment described above using a purified enzyme. Pre-incubation of purified cathepsin H with the library of compounds followed by ¹²⁵I-DCG-04 labeling resulted in a specificity profile that was remarkably similar to the profile observed for cathepsin B in crude extracts (FIG. 6C). While these two proteases are quite different in their biological functions, it is clear from these data that the two have similar inhibitor specificity in the S2 pocket.

Since it is unlikely that two distinct proteases will exhibit identical reactivity across a diverse set of inhibitors, it may be possible to use this information from positional scanning inhibitor libraries to generate “specificity fingerprints” for a series of well characterized proteases. Establishment of a database of protease inhibitor profiles could potentially be used to establish target identification by labeling of crude protein mixtures in the presence of compound libraries. Furthermore, extension of this methodology to longer, more diverse peptide substrate analogs may further accentuate the specificity differences of closely related protease species.

### Profiling Across Tissue Types

Having determined that both DCG-03 and DCG-04 were capable of covalently modifying multiple papain family proteases in extracts generated from several cell lines, we wanted to test the utility of these reagents for profiling protease activity patterns in various tissues. In this way, a crude map of protease activities can be created for each tissue and ultimately the identity of these major species can be determined by virtue of their reactivity towards the DCG-04 affinity probe.

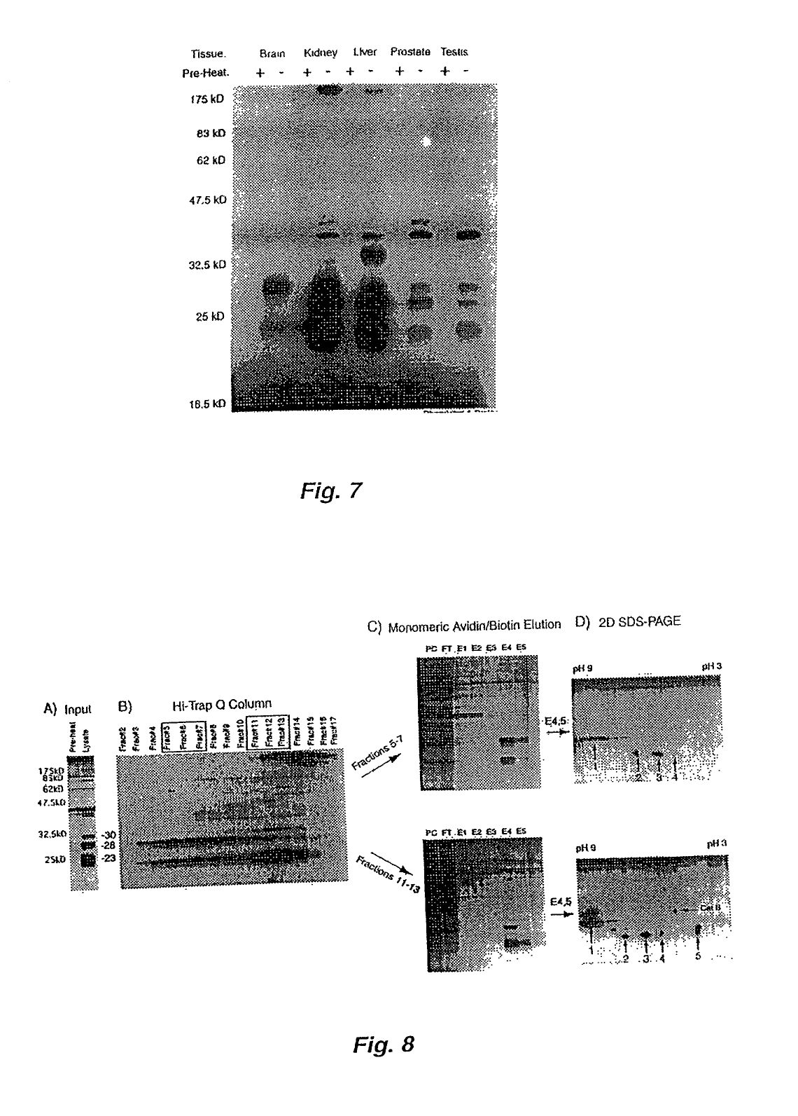

Samples of rat brain, kidney, liver, prostate and testis tissue were used to make crude homogenates at the reduced pH of the lysosome (pH 5.5). Samples were labeled with ¹²⁵I-DCG-04 and analyzed by SDS-PAGE/autoradiography (FIG. 7). The most intense labeling in the 20-30 kDa size range was observed for kidney and liver tissue consistent with the known protein processing functions of these organs. Comparison of the labeling profiles across tissue samples indicated that while some of the modified polypeptides were observed in multiple tissues at nearly identical intensities, several polypeptides showed increased or specific activity in a given tissue type. These data are consistent with the findings that cathepsin expression patterns and activities are differentially regulated across tissue types (Kominami et al. (1985) J. Biochem. 98: 87-93). In addition, the major species labeled by ¹²⁵I-DCG-04 were in the 20-30 kDa size range and are likely to be lysosomal cathepsins such cathepsin B, H and L. To confirm this hypothesis we chose rat kidney as a starting material for the affinity purification of targeted cysteine protease using DCG-04 as an affinity tag. The results of this purification are described below.

### Identification of DCG-04 Modified Proteins in Rat Kidney by Affinity Chromatography

Perhaps the greatest attribute of a functional proteomics tool is its ability to aid in the identification of targeted proteins. As shown above, rat kidney contains several polypeptides that were efficiently targeted by DCG-04 (FIG. 7). Three prominently labeled species of 23 kD, 28 kD, and 30 kD were identified in total kidney extract (FIG. 8A). When subjected to anion exchange chromatography, these polypeptides partitioned over a wide range of the elution gradient as determined by DCG-04 labeling of column fractions (FIG. 8B). Two pools of fractions were chosen based on differences in labeled protein composition. Fractions 7-9 contained predominantly the 23 and 28 kDa species and fractions 11-13 contained the 23, 28 and 30 kDa species. Modified proteins were affinity purified using a monomeric-avidin column that has a reduced binding affinity for biotin and thus the bound proteins could be competitively eluted with high concentrations of biotin (2 mM). The affinity column purified all DCG-04 modified polypeptides in both pools as visualized by SDS-PAGE and silver staining of eluted fractions (FIG. 8C). To further resolve DCG-04 modified polypeptides, peak fractions were concentrated, separated by 2D SDS -PAGE and visualized by silver staining (FIG. 8D).

The 30 kDa polypeptide (cat B) yielded a single spot near the acidic end of the gel, while the 28 kDa polypeptide (spot #1) resolved into a streak near the basic end of the gel. The 23 kDa band yielded three distinct spots ranging in p1 from acidic to basic (spots #2-5). All spots were excised from the gel and subjected to in-gel trypsin digestion, followed by peptide extraction and analysis by mass spectrometry. The protein amount in the 30 kDa spot was not sufficient for unambiguous identification based on MS data alone. Thus its identity was confirmed as cathepsin B by labeling of anion exchange column fractions with the cathepsin B specific label ¹²⁵I-MB-074 (Bogyo et al. (2000) Chem Biol., 7: 27-38) (data not shown).

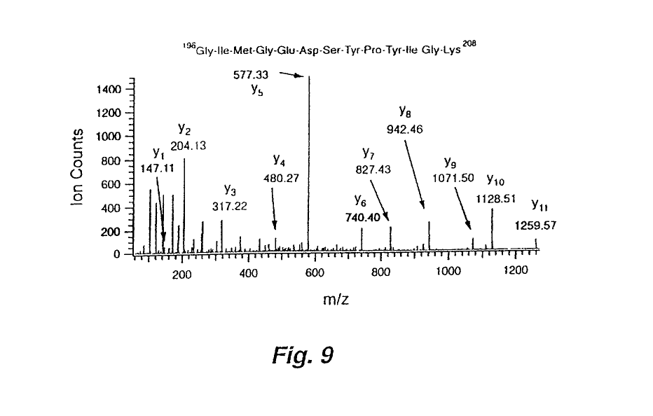

The tryptic mass fingerprint obtained for the 28 kDa band as well as two of the three 23 kDa spots (#2, #3) indicated the presence of cathepsin H. Furthermore, all three digests contained a MH⁺1429.7 peptide that was sequenced by low energy dissociation analysis (CID; FIG. 9). The resulting sequence, MGEDSYPYL/IGK (SEQ ID NO:2), unequivocally matched cathepsin H. The amino terminus of cathepsin H is heterogeneous, explaining the presence of multiple cathepsin H isoforms at similar molecular weights (Ishidoh et al. (1998) Biochem. Biophys. Res. Comm. 252: 202-207). In addition cathepsin H exists as both single chain and two-chain isoforms differing by about 5 kDa (Ishidoh et al. (1998) Biochem. Biophys. Res. Comm. 252: 202-207). Thus, spot #1 is likely to be the single chain form of cat H while spots 2 and 3 may represent heavy chain versions of the two-chain isoform.

The remaining 23 kDa spots (#4, #5) did not yield sequence data, however spot #5 was identified as cathepsin L based on the tryptic peptides observed in its digest, its size and pI. Thus, DCG-04 successfully identified the predominant active cysteine proteases in rat kidney as cathepsin B, H, and L in agreement with previous studies (Kominami et al. (1985) J. Biochem. 98: 87-93).

### Conclusion

The need for functional proteomics methods is becoming more important as genomics efforts complete the sequences of various organisms. Cravatt and co-workers have established the utility of a functional proteomics tool specific for the serine hydrolase family of proteases (Liu et al. (1999) Proc. Natl. Acad. Sci., USA, 96: 14694-14699). We show here that a general affinity label, DCG-04 and its radiolabeled counterpart ¹²⁵I-DCG-04 can be used to profile cysteine protease activities in crude extracts from cells and tissues, as well as throughout multiple stages of a physiological process. Diversification of the peptide portion of the inhibitor using solid-phase synthesis established the utility of small libraries of compounds for determining profiles of inhibitor specificity for both characterized and potentially novel enzymes. The information obtained from these libraries provides a starting point for the development of protease-specific inhibitors and also provides functional information about a protease target that may serve as a method for rapid identification of targets in crude protein mixtures. Furthermore DCG-04 can be used as an affinity purification reagent to aid in the identification of proteases selected by virtue of their reactivity towards our electrophilic probes. Target identification of proteases from crude extracts based on activity profiles will assist in the assignment of protein function as well as potentially identify new players in processes such as carcinogenesis. Finally, further diversification of these reagents is likely to extend their utility for the study of additional physiological processes that are regulated by proteolysis.

### Experimental Procedures

### Synthesis of DCG-04, DCG-03, and P2 Diverse Library

### Solution Phase Synthesis of Ethyl (2S,3S)-oxirane-2,3-dicarboxylate

The synthesis of this compound was according to the method described by Bogyo et al. (2000) Chem Biol., 7: 27-38.

### Solid-Phase Synthesis of DCG-04 and DCG-03

The details of the solid-phase synthesis are shown in FIGS. 2A and 2B. All resins and reagents were purchased from Advanced Chemtech (Louisville ,Ky.). Dry Fmoc-Rink amide resin (0.7 mmol/gram) was weighed into 1×10 cm columns (Waters). The columns were fitted with Teflon stopcocks and connected to a 20 port vacuum manifold (Waters) that was used to drain solvents and reagents from the columns. The resin was swelled using DMF. The Fmoc protecting group was removed (deprotected) by treatment with a 20% piperidine solution in DMF for 15 min. The resin was washed with 3×3mL. of DMF and 3×3 mL. of CH₂Cl₂.

Fmoc-Lys(biotin)-OH (100 mg, 70 μmol, 1 eq), DIC (11.4μl, 112 μmol, 15 eq), HOBT(15.1 mg, 112 μmol, 1.5 eq) were dissolved in 2 ml of DMF, added to the resin and the reaction was agitated for 1 hour. The resin was washed and the N-terminal Fmoc group was deprotected. Fmoc-6-aminohexanoic acid (74.2 mg, 210 μmol, 3 eq), DIC (21.4 μl, 210 μmol, 3 eq) and HOBT (28.4 mg, 210 μmol, 3 eq) were dissolved in 2 ml DMF and agitated with the resin for 1 hour, followed by washing and deprotection of the N-terminal Fmoc group (synthesis of DCG-03 leaves this step out). Fmoc-Tyr(But)-OH (160.8 mg, 350 μmol, 5 eq), DIC (35.6 μl, 350 μmol, 5 eq), and HOBT (47.2 mg, 350 μmol, 5 eq) were dissolved in 2 ml DMF and the reaction agitated for 1 hour followed by washing and N-terminal Fmoc group deprotection. Fmoc-Leucine (61.8 mg, 350 μmol, 5 eq), DIC (35.6 μl, 350 μmol, 5 eq), and HOBT (47.2 mg, 350 μmol, 5 eq) were dissolved in 2 ml DMF and the reaction agitated for 1 hour. The resin was washed followed by deprotection of the N-terminal Fmoc group. Ethyl (2S,3S)-oxirane-2,3-dicarboxylate (22.4 mg, 140 μmol, 2 eq), DIC (14.2 μl, 140 μmol, 2 eq), and HOBT (18.9 mg, 140 μmol, 2 eq were dissolved in 2 ml DMF and the reaction agitated for 1 hour. The resin was washed with 3×3 ml of DMF and 3×3 ml of CH₂Cl₂.

The inhibitors were cleaved from the resin using 1 mL of cleavage cocktail (95% TFA, 2.5% water, 2.5% triisopropylsilane). The mix was collected and the resin washed with 0.5 mL of fresh cleavage cocktail. Ice cold ether (15 ml ) was used to precipitate the product. The solid was collected and dissolved in a minimal amount of DMSO. The product was purified on a C18 reverse phase HPLC column (Waters, Delta-Pak) using a linear gradient of 0-100% water-acetonitrile. Fractions containing the product were pooled, frozen and lyophilized to dryness. The identity of the product was confirmed by mass spectrometry. Electrospray mass spectrum: [M+H] calc'd for DCG-03 C₃₇H₅₅N₇O₁₀S 791.0 found 791.0; calc'd for DCG-04 C₄₃H₆₆N₈O₁₁S 903.1 found 903.7.

A similar protocol was used to synthesize the P2 diverse library except that synthesis was performed using a 96 well manifold (Robbins Scientific). Synthesis was carried out on 20 mg of Rink resin per well and all coupling conditions were identical to those described above. Each of 18 natural amino acids (excepting cysteine and methionine) and including norleucine were coupled after addition of the amino hexanoic acid spacer group. All subsequent steps were performed as described above except peptides were used without HPLC purification due to the fact that products were found to be pure by HPLC analysis. Identity of products was confirmed by mass spectrometry. Electrospray mass spectrum: X=Ala calc'd [M+H] for C₄₀H₆₀N₈O₁₁S 862.0 found 861.9; Arg C₄₂H₆₆N₁₂O₁₁S 946.5 found 946.7; Asn C₄₁H₆₁N₉O₁₂S 905.0 found 904.9; Asp C₄₁H₆₀N₈O₁₃S 906.0 found 905.9; Glu C₄₂H₆₂N₈O₁₃S 920.0 found 919.8; Gln C₄₂H₆₃N₉O₁₂S 919.0 found 918.9; Gly C₃₉H₅₈N₈O₁₁S 848.0 found 847.7; His C₄₃H₆₂N₁₀O₁₁S 928.0 found 927.7; Ile C₄₃H₆₆N₈O₁₁S 904.1 found 904; Leu C₄₃H₆₆N₈O₁₁S 904.1 found 904.0; Lys C₄₃H₆₇N₉O₁₁S 919.0 found 919.0, C₄₆H₆₄N₈O₁₁S 938.0 found 937.8; Pro C₄₂H₆₂N₈O₁₁S 888.0 found 877.8; Ser C₄₀H₆₀N₈O₁₂S 878.0 found 877.8; Thr C₄₁H₆₂N₈O₁₂S 892.0 found 892.0; Trp C₄₈H₆₅N₉O₁₁S 977.1 found 976.7; Tyr C₄₆H₆₄N₈O₁₂S 954.1 found 953.8; Val C₄₂H₆₄N₈O₁₁S 890.0 found 890.0 found 890.0; Nle C₄₃H₆₆N₈O₁₁S 904.1 found 903.9.

### Radiolabeling of Inhibitors

All compounds were iodinated and isolated using the protocol described by Bogyo et al. (2000) Chem Biol., 7: 27-38.

### Preparation of Cell and Tissue Lysates

Tissues were dounce-homogenized in buffer A (50 mM Tris pH 5.5, 1 mM DTT, 5 mM MgCl₂, 250 mM sucrose) and extracts centrifuged at 1,100×g for 10 min at 4° C. The resulting supernatant was centrifuged at 22,000×g for 30 min at 4° C. and final supernatants used for all labeling experiments. Cells were lysed using glass beads (<104 microns) in buffer A and supernatants centrifuged for 15,000×g for 15 min at 4° C. The total protein concentration of the final supernatants (soluble) was determined by BCA protein quantification (Pierce).

Labeling of Lysates with ¹²⁵I-DCG-04, ¹²⁵I-DCG-03, and ¹²⁵I-MB-074

Equivalent amounts of radioactive inhibitor stock solutions (approx. 10⁶ cpm per sample) were used for all labeling experiments. Samples of lysates (100 μg total protein in 100 μL buffer; 50 mM Tris pH 5.5, 5 mM MgC12, 2 mM DTT) were labeled for 1 hour at 25° C. unless noted otherwise. Samples were quenched by dilution of 4×SDS sample buffer to 1×(for 1D SDS-PAGE) or by dissolving urea to a final concentration of 9.5 M (for 2D SDS-PAGE).

### Gel Electrophoresis

One-dimensional SDS-PAGE, two-dimensional IEF gels were performed as described (Bogyo et al. (1998) Chem Biol., 5: 307-320).

### SDS/PAGE-western Blotting Detection of and Auto-radiography of DCG-04 Modified Proteins.

Quenched DCG-04 labeled samples were separated by SDS/PAGE (100 μ/lane) and transferred to nitrocellulose using semi-dry apparatus. Membranes were blocked using phosphate buffered saline (PBS) and 5% (w/v) dry milk for 30 min at 25° C. Blots were washed briefly with PBS/0.2% Tween (PBS-Tween) and treated with avidin-horseradish peroxidase conjugate (VectaStain) in PBS-Tween for 30 min. at 25° C. Blots were washed three times with PBS-Tween, treated with ECL reagents (Amersham), and exposed to film.

### Competition Labeling Experiments

Lysates from the dendritic cell line DC2.4 were prepared at pH 5.5 as described above. Purified cathepsin H was purchased from Calbiochem (San Diego, Calif.). Samples of lysates (100 μg total protein in 100 μL buffer B; 50 mM Tris pH 5.5, 5 mM MgC12, 2 mM DTT) or purified cathepsin H (1 μg protein in 100 μL buffer A) were preincubated with 50 μM of each library member (diluted from 5 mM DMSO stocks) for 2 hrs at room temperature. Samples were then labeled by addition of either ¹²⁵I-DCG-04 or ¹²⁵I-MB-074 to each sample followed by further incubation at room temperature for 1 hour. Samples were quenched by the addition of 4×sample buffer to 1×followed by boiling for 5 minutes. Samples were analyzed by SDS-PAGE followed by autoradiography.

### Preparation of Mouse Carcinoma Cell Lines

Mouse melanoma cell lines were prepared by a single topical application of 25 μg of the chemical mutagen dimethylbenzanthracene (DMBA) to the skin of mice followed by biweekly application of 100 μM of the tumor promoter, TPA, over an extended period of time essentially as described (Bremner and Balmain (1990) Cell 61: 407-417; Burns et al. (1991) Oncogene 6: 2363-2369; Haddow et al. (1991) [published erratum appears in Oncogene 1991 December; 6(12):2377-8]. Oncogene 6: 1465-1470).

### Protein Identification of DCG-04 Modified Proteins

A soluble fraction of rat kidney lysate (80 mg total protein) was diluted into anion exchange starting buffer (50 mM Tris, 50 mM NaCl, pH 9.0). The lysate was applied to a HitrapQ anion exchange column (Amersham Pharmacia Biotech) and eluted using a linear gradient of 0.05-1M NaCl, pH 9. An aliquot from each fraction (50 μL) was incubated with 50 μM DCG-04 at 25° C. for 1hr and analyzed on a 12.5% SDS/PAGE gel followed by affinity blotting as described above.

The fractions containing peak labeling of the 25 kD-30 kD bands were pooled and DCG-04 was added to a final concentration of 50 μM. Pools were incubated at 25° C. for 2 hours and then 12 hours at 4° C. Unbound inhibitor was removed and buffer was exchanged with PBS using a PD-10 column (Pharmacia). Samples were applied to a monomeric-avidin column (1 ml bed volume; Pierce) and the column was washed with 6×1 ml fractions of 1M NaCl. Bound proteins were eluted with 0.5 ml fractions of 2 mM Biotin/100 mM NH₄HCO₃ buffer. All wash and eluent fractions were analyzed by SDS/PAGE and silver staining. The fractions containing the labeled 25-30 kD bands were pooled, the volume reduced by lyophilization and solid urea added to 9.5 M along with BME to 5%, NP-40 to 2%, pH 5-7 ampholytes to 1.6% and pH 3.5-10 ampholytes to 0.4%. Samples were applied to IEF tube gels and electrophoresed at 1000V for 13 hours followed by separation in the second dimension on 12.5% SDS-PAGE gels.

The resulting gels were fixed in 12% acetic acid/50% methanol stained with silver according to reported protocols (Bogyo et al. (1998) Chem Biol., 5: 307-320). Spots were excised, digested with trypsin, and the peptide molecular weight measurements were carried out by MALDI-MS (PE Voyager DESTR). Sequence determination was performed on a quadrupole time-of-flight hybrid tandem mass spectrometer (PE QSTAR) equipped with a Protome nanospray source. This instrument affords high resolution and accuracy for mass measurement and the CID data obtained allowed unambiguous sequence determination. Database searches were performed using the Protein Prospector software package (http://prospector.ucsf.edu/).

### Example 2

### Chemical Approaches for Functionally Probing the Proteome

### Introduction

Over the past few years the complete genome sequences of multiple organisms have been determined. These efforts have been followed by the annotation of genes that code for all proteins of an organism's proteome. While this information is likely to provide valuable information, a great deal of effort is required to define the function of individual gene products. Informatics techniques have been developed to assign function to individual genes by analyzing patterns of co-inheritance throughout multiple organisms (Marcotte et al.(1999) Nature 402: 83-86; Eisenberg et al. (2000) Nature 405: 823-826). Furthermore, analysis of genome-wide changes in transcription in response to different stimuli allows clustering of genes of similar function based on transcriptional co-regulation (Eisen et al. (1998) Proc. Natl. Acad. Sci., USA, 95: 14863-14868). While these methods help to broadly classify proteins into families based on predicted function, the assignment of functions to specific members within a large enzyme family remains a difficult task.

Proteomics approaches address some of the gaps in genomics methodologies by profiling and identifying bulk changes in protein levels (Dove (1999) Nat Biotechnol 17: 233-236; Pandey and Mann (2000) Nature 405: 837-846). However, these methodologies only provide information for abundant proteins while proteins with difficult biochemical properties (i.e. membrane proteins) are often excluded from analysis. Moreover, for most enzymes, their activity, and therefore their function, is regulated by a complex set of post-translational controls. Therefore, even proteomic profiles in many cases provide an incomplete picture of how enzymes are functionally regulated (Gygi et al. (1999) Molecular and Cellular Biology 19: 1720-1730).

Classical genetic approaches are tried and true methods to assign functions to specific gene products. In many biological systems it is possible to disrupt a desired gene and assess the resulting phenotype. However, this process is often tedious and in cases where multiple related proteins have similar functions, compensation adjustments make the resulting phenotype difficult to interpret.

To circumvent these problems, small molecules can be used to manipulate the activity of protein targets (Stockwell (2000) Trends Biotechnol 18: 449-455; Schreiber (1998) Bioorg Med Chem 6: 1127-1152). This “chemical genetic” approach makes use of libraries of small molecules to screen for compounds that perturb a given biological process. The resulting ‘hits’ can then be used to begin to assign function to specific enzyme or protein targets. However, the utility of this process is limited by the difficult task of identifying the relevant target of the small molecule.

In the case of traditional drug discovery, small molecule libraries are screened against a single pre-defined target. Lead compounds are often identified from large chemical libraries using an in vitro assay. While many of these compounds are effective against the purified target, little is usually known about their selectivity in a crude proteome. Therefore, a method that allows screening for small molecule inhibitors in cell and tissue extracts or intact cells would allow identification of lead compounds based on multiple criteria such as potency, selectivity and cell permeability. Furthermore, compounds could be screened against entire enzyme families thereby increasing the chances of identifying useful compounds for therapeutic intervention.

We have developed chemically reactive affinity probes that can be used to (i) identify the members of a given enzyme family within a proteome (ii) determine the relative activity levels of individual family members (iii) localize active enzymes within a cell (iv) screen small molecule libraries directly in crude protein extracts for inhibitors that can ultimately be used to determine biological functions of specific target enzymes. In this study, we have chosen to focus on the papain family of cysteine proteases for several reasons. Firstly, these proteases are synthesized as inactive zymogens that are activated post-translationally (Cygler et al. (1996) Structure 4: 405-416; Coulombe et al. (1996) Embo Journal 15: 5492-5503). Their activity can also be regulated by interaction with macro-molecular inhibitors resulting in transcription/translational profiles that provide only limited information regarding their functional regulation. Secondly, the papain family is composed of many closely related family members whose functions are poorly defined (Chapman et al. (1997) Annu. Rev. Physiol. 59: 63-88). Thirdly, many small molecule covalent inhibitors of this class of enzyme have been developed that can be used for probe design (see, Shaw (1994) Meth. Enzymology 244: 649-656, and refs therein). Finally, these enzymes have been found to play an important role in many disease conditions such as cancer (Yan et al. (1998) Biol. Chem., 379: 113-123), osteoporosis (Gelb et al. (1996) Science 273: 1236-1238), asthma (Chapman et al. (1997) Annu. Rev. Physiol. 59: 63-88), and rheumatoid arthritis (Iwata et al. (1997) Arthritis and Rheumatism 40: 499-509) making them a potential important class of enzymes for drug development.

### Results and Discussion

### Probe Design and Application to Pure Enzymes, Crude Homogenates and Intact Cells

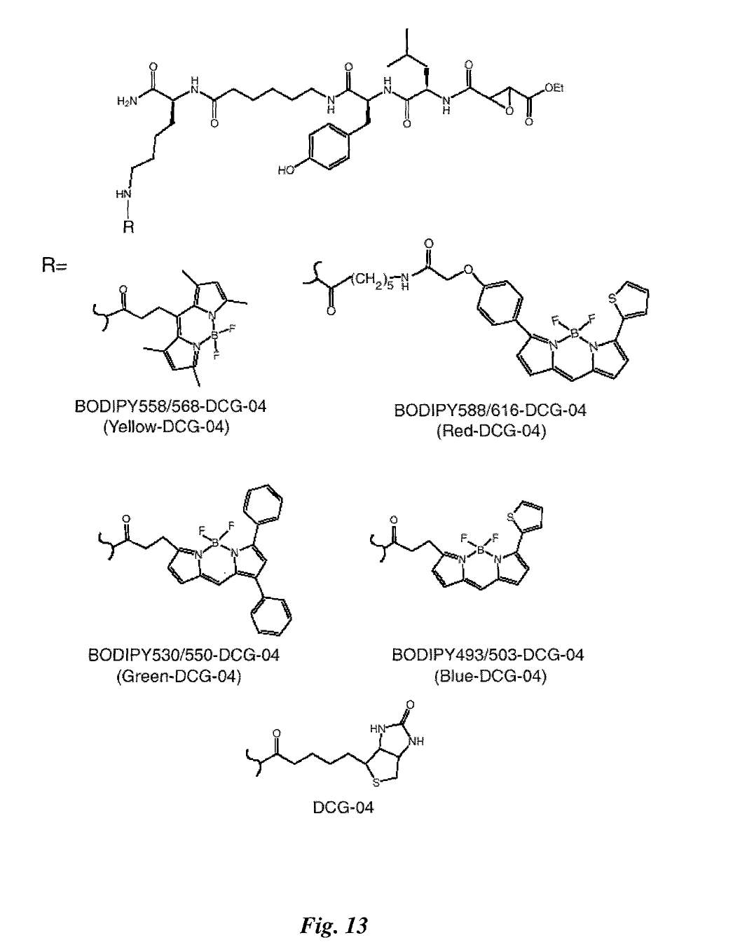

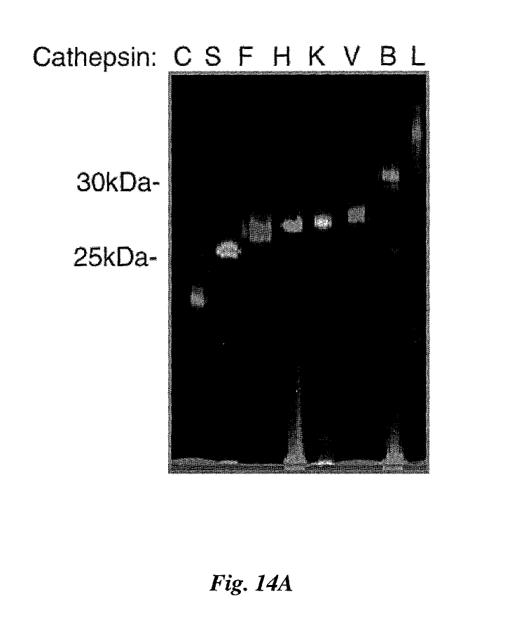

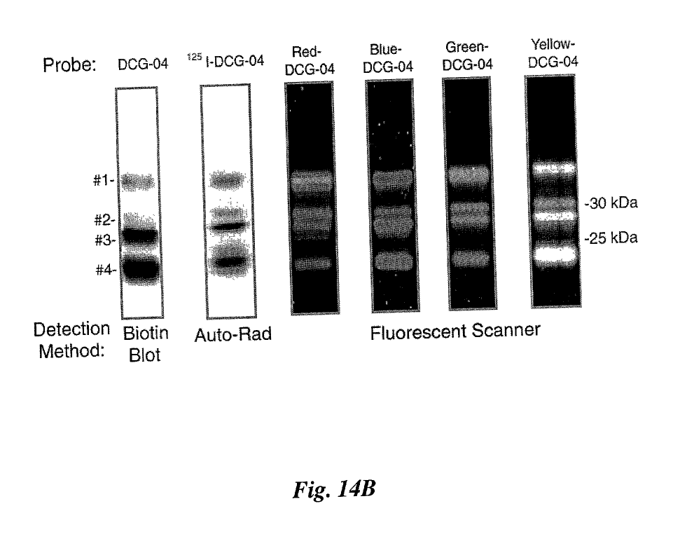

Several laboratories have developed small molecule electrophiles that show class-specific reactivity towards nucleophilic active site residues of several different enzyme families. These include serine (Liu et al. (1999) Proc. Natl. Acad. Sci., USA, 96: 14694-14699; Kidd et al. (2001) Biochem., 40: 4005-4015) and cysteine (Bogyo et al. (2000) Chem Biol 7: 27-38; Greenbaum et al. (2000) Chem Biol 7: 569-581; Faleiro et al. (1997) Embo Journal 16: 2271-2281) hydrolases as well as aldehyde dehydrogenases (Adam et al. (2001) Chem Biol 8: 81-95). In each case, electrophiles have been designed that exhibit broad irreversible reactivity for enzyme family members, while remaining relatively inert towards free-circulating nucleophiles such as thiols, hydroxyls and amines. The resulting activity-based probes (ABPs) can be used to covalently label specific target enzymes within the complex mixture of proteins from a cell or tissue sample. Our laboratory has developed probes based on the structure of the natural product E-64 19. These ABPs can be used to affinity label papain family cysteine proteases. They also allow rapid purification of labeled proteases by virtue of incorporation of a biotin affinity tag. Here we have used the core peptide epoxide analog of E-64 to create four fluorescently labeled ABPs for papain family cysteine proteases (FIG. 13).

These probes incorporate four different fluorescent moieties, each with non-overlapping excitation and emission spectra, allowing for multiplexing of probes. Four BODIPY analogs were chosen based on the excitation and emission wavelengths of fluorophores commonly used in DNA sequencing protocols. We reasoned that it should be possible to visualize and quantify fluorescently labeled proteins using a standard DNA sequencing apparatus equipped with a high intensity laser. FIG. 14A shows the gel image that results from incubation of eight different purified papain family cysteine proteases with each of the four fluorescent ABPs followed by analysis on an ABI 377 DNA sequencer. Using these probes, it is possible to load all eight proteases in a single gel lane and distinguish each, based on differences in molecular weight and emission wavelength of fluorescent labels.

The same four probes were next used to profile the repertoire of papain family proteases within a complex protein mixture derived from a tissue homogenate. FIG. 14 shows the profiles of cysteine proteases in total rat liver homogenates obtained by labeling with the biotinylated probe DCG-04, the radiolabeled version of DCG-04, and the four fluorescent analogs of DCG-04. All ABPs labeled the same four predominant protease species with only slight differences in relative intensities observed for each probe. These results suggest that the presence of structurally diverse labeling groups at the distal affinity site of the molecules had little effect on a compound's ability to covalently modify its targets.

Since covalent modification of target proteases by the ABPs requires modification of the active site thiol nucleophile, labeling intensities can be used as an indirect measure of enzymatic activity. Thus, unlike antibodies that can only be used to monitor bulk levels of specific proteins, these reagents allow analysis of changes in levels of enzymatic activity. In the past, our laboratory has used these reagents to follow activity of cysteine proteases during processes such as tumor progression and cell invasion (Bogyo et al. (2000) Chem Biol 7: 27-38). These newly developed ABPs therefore provide an efficient method for monitoring changes in protease activities within a proteome.

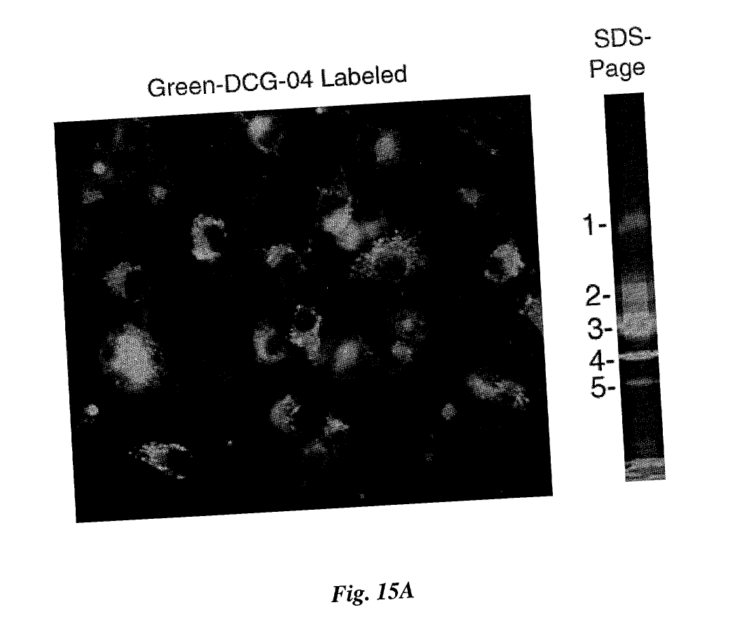

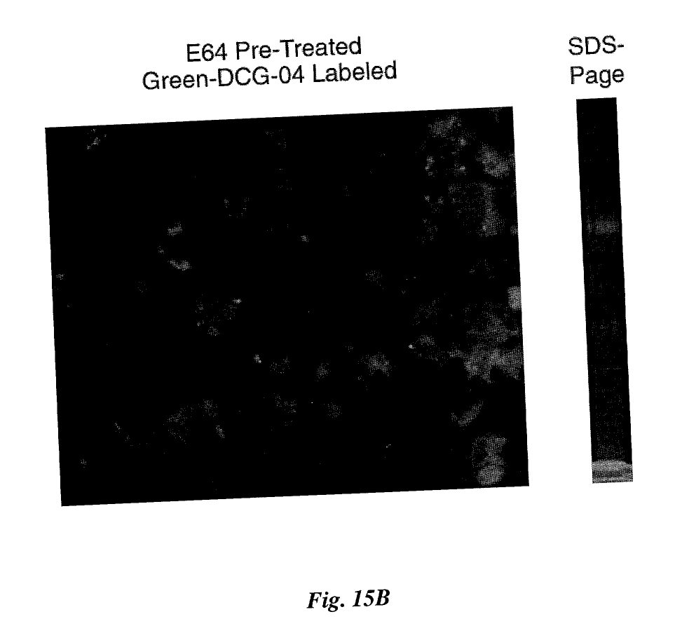

Since the fluorescent probes are cell permeable they make ideal tools for imaging of protease activity in intact cells or tissue sections. FIG. 15 shows the dendritic cell line DC2.4 either directly labeled in situ with green-DCG-04 or pre-treated with E-64 and then labeled with the fluorescent probe. Cells directly treated with the green ABP showed a fluorescence staining pattern characteristic of lysosomal compartments. Cells that had been pre-treated with E-64 showed diffuse fluorescence throughout the cytosol, likely due to residual free probe that failed to be washed away. The cells were collected after imaging, lysed and analyzed by SDS-PAGE and fluorescence detection. The resulting profiles indicated that multiple protease species were labeled by the fluorescent probe and that these proteases were completely inhibited by pre-treatment of cells with E-64. Thus the fluorescent staining observed in the non-pretreated cells represents the localization of active papain family cysteine proteases. This method is likely to be applicable to tissue samples and may serve as a convenient way to image protease activities in tissues derived from important clinical samples such as solid tumors.

### Using ABPs to Generate Inhibitor Specificity Profiles for Papain Family Proteases

The concept of classifying enzyme family members based on structure-activity relationship homology (SARAH) has been proposed as an alternative to classification methods based on sequence homology (Frye (1999) Chem Biol 6: R3-7). Using this approach, large enzyme families can be classified based on their reactivity towards small molecule ligands thereby aiding the process of functional analysis and drug design. ABPs serve as ideal tools for the rapid analysis of SARAH between closely related enzyme family members. Furthermore, ABPs allow SARAH analysis directly in crude protein extracts thereby allowing the classification of potentially novel target enzymes.

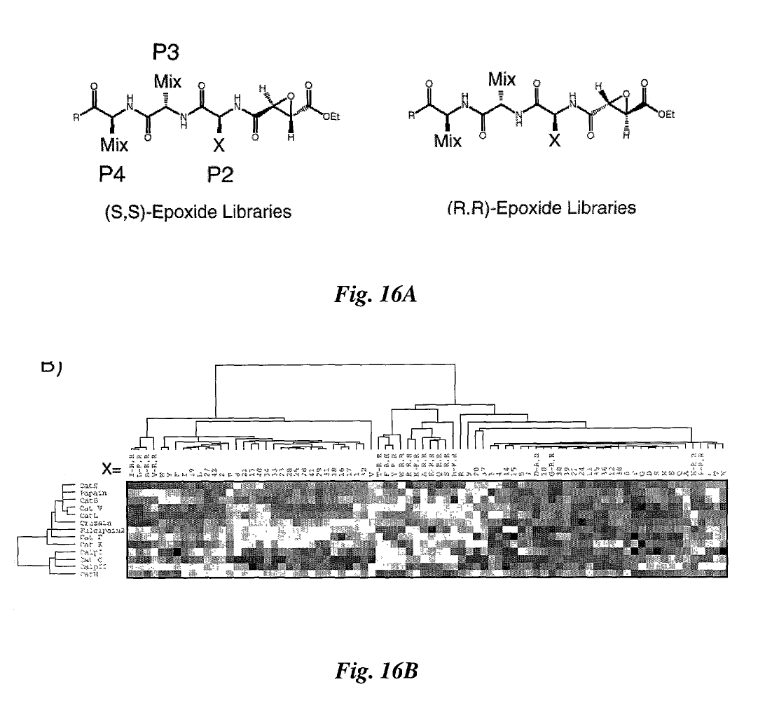

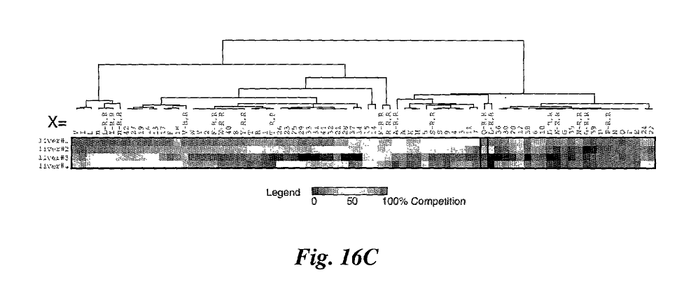

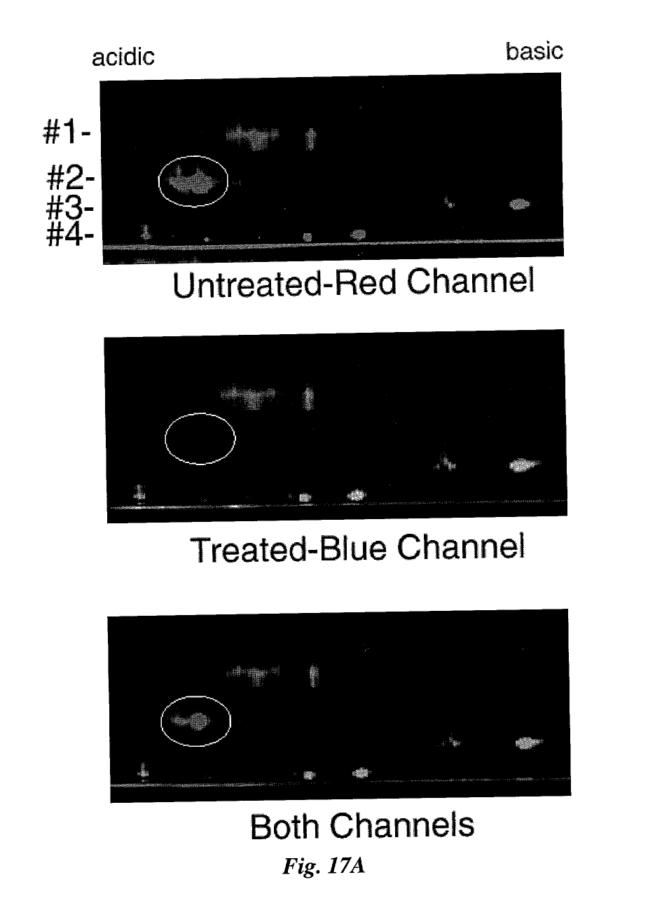

To begin SARAH analysis of papain family enzymes, a series of small molecule libraries were designed based on a core peptide backbone coupled to the epoxide electrophile contained in the DCG-04 probes (FIG. 16A). Initially, positional scanning libraries (PSLs) were synthesized in which a single amino acid position was scanned through a series of natural and non-natural amino acids, while the remaining two positions were coupled with a mixture of all possible natural amino acids (minus cysteine and methionine and including norleucine). The resulting sub-libraries were composed of 361 members each. Scanning of constant amino acids at the P3 and P4 positions through all natural amino acids indicated that these elements did not significantly contribute to selectivity of inhibitor binding (data not shown). Therefore, only data compiled for scanning of the constant P2 position are presented. To increase the diversity of the small molecules in the PSLs we included 42 hydrophobic non-natural amino acids as building blocks (see structures in Table 1). In addition, each of the natural amino acids was coupled to the mirror-image enantiomeric form of the epoxide (2R, 3R vs. 2S, 3S). Previous work indicates that this change in stereochemistry favors binding of the inhibitors on the prime side of the active site resulting in more diversity in our libraries (Schaschke et al. (1997) Bioorganic and Medicinal Chemistry 5: 1789-1797).

TABLE 1
Non-natural amino acids used in PSLs.

| cmpd # | Amino Acid | Structure |
|---|---|---|
| 1 | (2furyl)alanine | 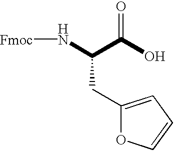 |
| 2 | (2thienyl)alanine | 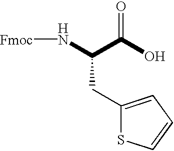 |
| 3 | 2pyridylAla | 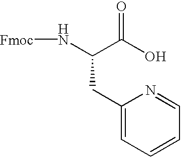 |
| 4 | 1amino1cyclohexane carboxylic acid | 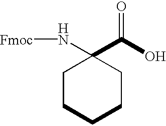 |
| 5 | 1amino1cyclopentanecarboxylic acid | 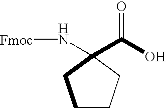 |
| 6 | 2-Abz | 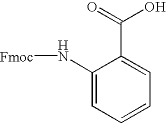 |
| 7 | 3Abz | 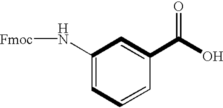 |
| 8 | 2Abu | 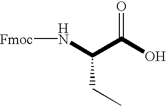 |
| 9 | 3amino3phenylpropionic acid | 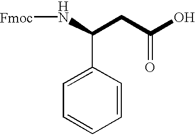 |
| 10 | dehydroAbu | 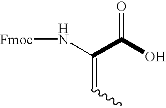 |
| 11 | ACPC | 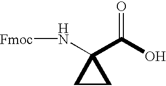 |
| 12 | Arb | 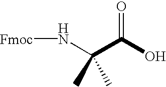 |
| 13 | AllylGly | 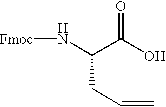 |
| 14 | Amb | 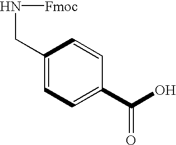 |
| 15 | Amc | 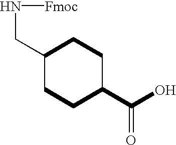 |
| 16 | Bip | 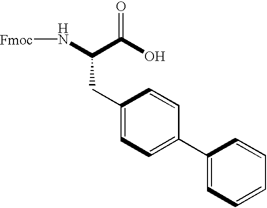 |
| 17 | Bpa | 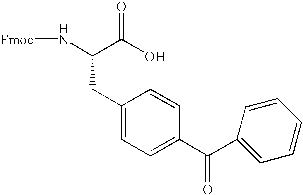 |
| 18 | Cba | 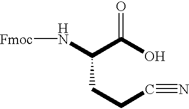 |
| 19 | Cha | 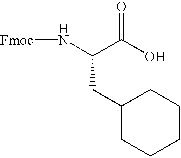 |
| 20 | deltaLeu | 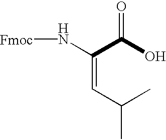 |
| 21 | deltaVal | 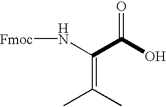 |
| 22 | Hyp | 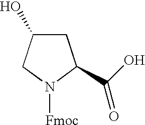 |
| 23 | IgI | 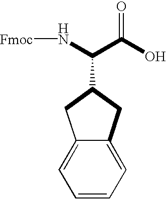 |
| 24 | Inp | 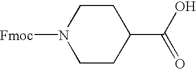 |
| 25 | 1-Nal | 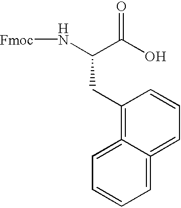 |
| 26 | 2-Nal | 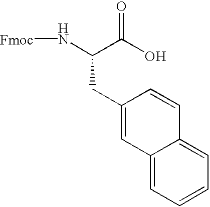 |
| 27 | Nva | 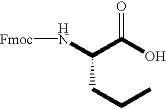 |
| 28 | 4-nitroPhe | 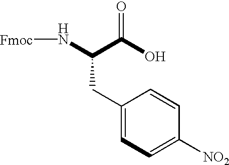 |
| 29 | 4MethylPhe | 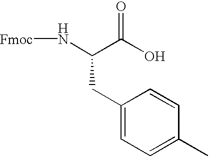 |
| 30 | 4Methyl-DPhe | 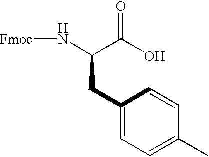 |
| 31 | Phe(pl) | 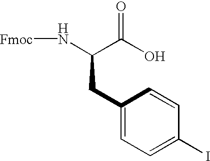 |
| 32 | Phe4NH(Boc) | 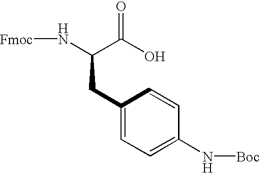 |
| 33 | hPhe | 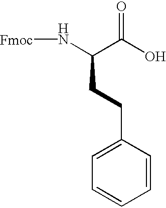 |
| 34 | Phg | 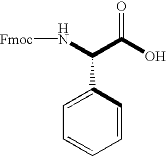 |
| 35 | pip | 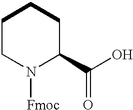 |
| 36 | Dpip | 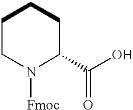 |
| 37 | propargylglycine | 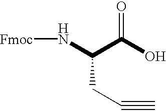 |
| 38 | Thz | 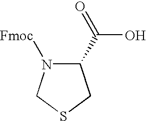 |
| 39 | Tic | 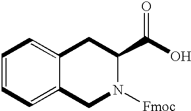 |
| 40 | Tle | 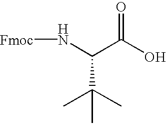 |
| 41 | 3-NitroTyr | 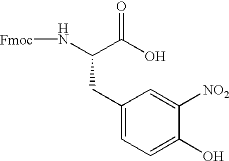 |
| 42 | leu | 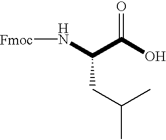 |

PSLs were first screened against thirteen purified papaine family enzymes (FIG. 16B). Potency was assessed by pretreatment of pure enzymes with each library followed by labeling with ¹²⁵I-DCG-04 and analysis by SDS-PAGE and autoradiography. The ability of each library to block active site labeling by DCG-04 was measured as the percentage of competition relative to an untreated control. The resulting values were visualized using software developed by Eisen and co-workers designed to analyze data generated from micro-array analysis (Eisen et al. (1998) Proc. Natl. Acad. Sci., USA, 95: 14863-14868). This software assigns a color to numerical competition values and allows clustering of profiles based on similarities across diversity positions (X-axis) and enzyme family members (Y-axis). The resulting “clustergram” is shown in FIG. 16B.

Clustering data throughout the constant amino acid residues grouped the data, such that residues that showed overall poor binding to all targets were positioned to the tight and residues that showed universal strong binding were positioned to the left. The remaining residues in the middle of the clustergram showed some degree of selectivity for individual enzymes. The results from the clustering indicate that the non-natural amino acids and natural amino acids linked to the (R,R) enantiomer of the epoxide provided the greatest target selectivity.

Similarly, clustering the data across the Y-axis grouped the enzymes based on similarities in specificity fingerprints or SARAHs. The clustering therefore allowed enzymes to be classified based on active site topology, as reflected in their ability to bind sets of small molecule ligands. The results from the cluster indicate that, in general, enzymes that are closely related by sequence homology (i.e. Cat V and Cat L) tend to cluster together based on specificity fingerprints. However, in some cases enzymes with close sequence homology showed markedly different specificity profiles with respect to inhibitor/substrate binding (i.e. Cat C and Cat B). Thus, specificity profiling provides a potentially more informative means for grouping related enzymes. Furthermore, it is possible to classify unknown protease species from crude extracts by generating fingerprints of targets and comparing them to fingerprints of well-characterized family members. This technique allows rapid classification of unknown enzymes and provides useful information for the design of small molecule inhibitors targeted for them.

### Using ABPs to Screen for Selective Inhibitors of Papain Family Cysteine Proteases in Crude Tissue Extracts

Perhaps the most powerful attribute of ABPs is their ability to facilitate screening of small molecule inhibitors against complete enzyme families without the need to first identify, clone and express individual targets. Furthermore, the data that is obtained from the screening process provides information not only regarding potency of the potential lead compounds, but also regarding selectivity of the compounds in a physiologically relevant sample that contains many closely related family members. To demonstrate the utility of this approach we performed screening of our PSLs in crude liver extracts (FIG. 16C). Specificity profiles for each of the major protease species labeled by DCG-04 were obtained. The resulting clustergram indicated that several residues which clustered to the center of the profile could be selected that would confer unique specificity for an individual protease species in the extract. Therefore, this method yielded interesting lead compounds using a relatively small number of libraries (˜80) with limited structural diversity. A similar screen of a larger, more structurally diverse small molecule library is likely to provide an even greater number of inhibitor leads. Given the relative ease of screening and the abundance of the protein extracts, such a large-scale screen is clearly accessible using this methodology.

### Profiling Changes in Protease Activities Upon Addition of Selective Small Molecule Inhibitors

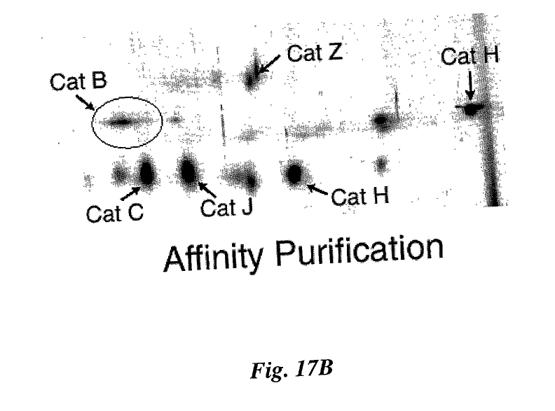

Analysis of the library data from screening of liver extracts indicated that several PSLs showed selective binding to a single protease. We chose to focus on the constant P2 glutamine (R,R) epoxide library because of its high degree of selectivity for protease #2 in the extract. Liver extracts were either directly labeled with the red-DCG-04 probe or treated with the library and then labeled with the blue-DCG-04 probe. The samples were then combined and subjected to a first dimension of isoelectric focusing followed by analysis by SDS-PAGE in the second dimension using the DNA sequencer (FIG. 17A). This method allowed analysis of multiple channels of data in a single gel that could be merged to determine changes in activity of each protease species in the presence of the inhibitor library. The resulting 2D profile unambiguously demonstrated that the glutamine (R,R) library specifically binds to the active site of a single protease (spot #2) as indicated by loss of labeling in the blue channel.

To determine the identity of the protease selectively targeted by the small molecule library, we used the biotin tagged DCG-04 to perform a single-step affinity purification of all labeled proteases from liver extracts. The resulting silver stained 2D profile shows that all fluorescently labeled protease could be rapidly purified from the crude extract and correlated with the labeling profiles (FIG. 14B). The silver stained spot corresponding to spot #2 was excised and identified as cathepsin B by LC-MS-TOF CID sequencing. Furthermore, several other cathepsin family members including cathepsins Z, H, C and J were identified by this method.

### Design of Selective Inhibitors Based on Library Screening Data

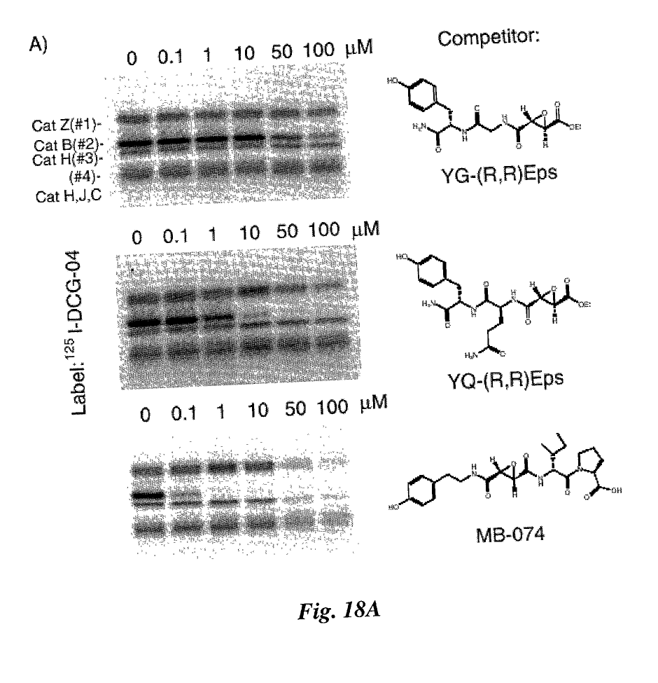

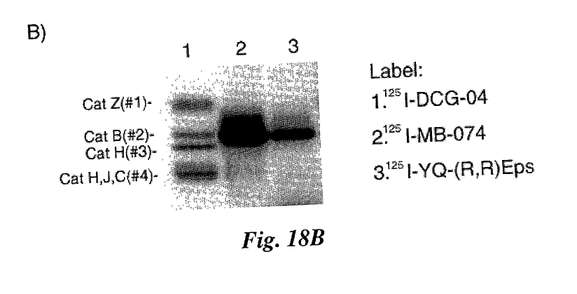

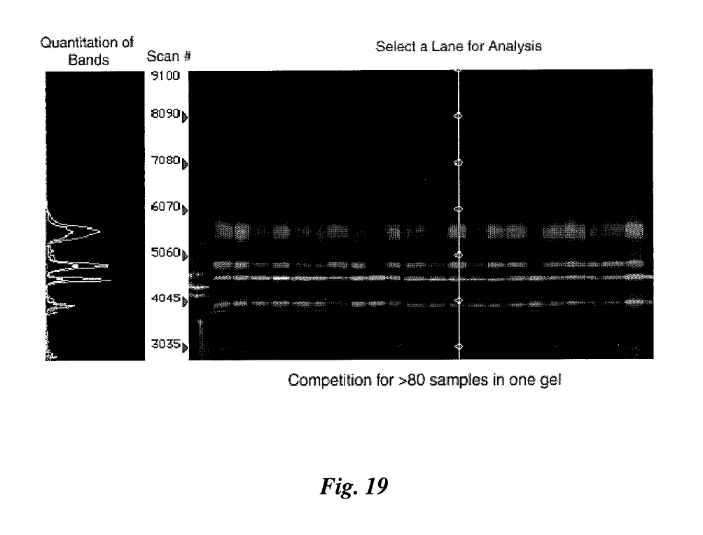

Using information from the scanning of our PSLs, we synthesized several single compounds designed to validate the library approach. In all cases a P3 tyrosine was included as a site for radio-iodination and the P2 residue was chosen based on target selectivity. P2 glutamine attached to the (R,R) epoxide inhibitor (YQ-(R,R)-Eps) was chosen because of its selectivity for cathepsin B in the extract and P2 glycine was chosen as a negative control. The cathepsin B specific ABP MB-074 (Bogyo et al. (2000) Chem Biol 7: 27-38) was used as a control for comparison with YQ-(R,R)-Eps. Compounds were added to extracts over a wide concentration range and activity for each target was assessed by labeling with ¹²⁵I-DCG-04 (FIG. 18A). As expected, YQ-(R,R)-Eps and MB-074 selectively blocked labeling of the cathepsin B band (#2) while GR-(R,R)-Eps showed little or no inhibition of all of the proteases. The newly developed cathepsin B inhibitor was also radioiodinated and used to label liver homogenates (FIG. 18B). The labeling profile was compared to the profiles for the cathepsin B-specific probe ¹²⁵I-MB-074 and the generally reactive probe ¹²⁵I-DCG-04. YQ-(R,R)-Eps, like MB-074, showed selective labeling of the band identified as cathepsin B. We conclude that it is possible to rapidly identify a structurally distinct class of cathepsin B selective inhibitors by screening of libraries of limited complexity. The resulting lead compound, while not excessively potent, now serves as a template for the design of optimized inhibitors that are distinct from the CA-074 class of cell impermeable cathepsin B inhibitors. No doubt this approach could also be used to selectively target other cathepsin family members through a more extensive library screening effort.

### Conclusions

In summary, we have developed tools to identify families of related enzymes within a complex proteome. These tools can be used to determine relative activity levels of these enzymes and to visualize their localization in live cells. These tools also allow rapid design and screening of small molecule inhibitors for select targets. In the current study we successfully identified a new cathepsin B selective inhibitor by screening of a small set of libraries in crude liver extracts. Furthermore, we have developed a general method for rapid analysis of large data sets generated from library screening of multiple targets in crude cell extracts. This approach allows rapid comparison of inhibitors as well as targets based on similarities in structure-function relationships. This general functional proteomic method, although applied here to papain family proteases, can also be used for a wide range of enzyme families through design and synthesis of new families of class-specific affinity probes.

### Materials and Methods

### Synthesis Protocols

### Synthesis of Ethyl (2S,3S)-oxirane-2,3-dicarboxylate and Ethyl (2R,3R)-oxirane-2,3- dicarboxylate and DCG-04.

The synthesis of (2R,3R)-oxirane-2,3-dicarboxylate is identical to that reported for the (2S,3S) isomer (Bogyo et al. (2000) Chem Biol 7: 27-38). The synthesis of DCG-04 is reported in Greenbaum et al. (2000) Chem Biol 7: 569-581.

### Synthesis of BODIPY558/568-DCG-04, BODIPY 588/616-DCG-04, BODIPY530/550-DCG-04, and BODIPY493/503-DCG-04.

All fluorophores where purchased from Molecular Probes (Eugene, Oreg.). A free amino version of DCG-04 was synthesized by replacing the terminal biotinylated lysine with lysine using the reported synthesis protocols for DCG-04 (Greenbaum et al. (2000) Chem Biol 7: 569-581). Free amino DCG-04 (6 mg, 8.8 mmol, 1.5 eq) and either BODIPY558/56-OSu (3.0 mg, 6.0 mmol, 1.0 eq), BODIPY 588/616-OSu (1.0 eq), BODIPY530/550-OSu (1.0 eq), or BODIPY493/503-OSu (1 eq) were dissolved in 100 ml DMSO. Diisopropylethylamine was then added (12.0 mmol 2.0 eq). The reaction was monitored by high performance liquid chromatography (HPLC). After 2 hours the product was purified on a Cis reverse phase BPLC column (Waters, Delta Pak) using a linear gradient of 0-100% water-acetonitrile. Fractions were pooled and lyophilized to dryness. The identity of the product was confirmed by mass spectrometry. Electrospray mass spectrum: [M+H] calculated for BODIPY558/568-DCG-04 C₄₉H₆₉BF₂N₈O₁₀ 979.5 ,found 978.5, BODIPY 588/616-DCG-04 C₆₀H₇₆BF₂N₉O₁₂S 1196.5 found 1197.0, BODIPY530/550-DCG-04 C₅₇H₆₉BF₂N₈O₁₀ 1075.5 found 1075.0, and BODIPY493/503-DCG-04 C₄₉H₆₃BF₂N₈O₁₀ S 1005.4, found 1004.5.

### Synthesis of Positional Scanning Libraries

Synthesis of the P2 constant PSL library was performed using a 96 well manifold (FlexChem, Robbins Scientific). Each library was constructed using a constant amino acid at the P2 position and an isokinetic mixture of all natural amino acids (minus cysteine and methionine plus norleucine) at the variable position. The isokinetic mixture was created using a ratio of equivalents of amino acids based on their reported coupling rates (Ostresh et al. (1994) Biopolymers 34: 1681-1689). The total mixture was adjusted to ten-fold excess total amino acids over resin load. For constant positions, a single amino acid was coupled using ten-fold excess. In addition to the natural amino acids, a set of 42 non-natural hydrophobic amino acids were also used for the constant P2 position (see supplemental materials). Couplings were carried out using Diisopropylcarbodiimide (DIC) and Hydroxybenzatrazole (HOBT) under standard conditions for solid phase peptide synthesis. Libraries and single components were cleaved from the resin by addition of 90% trifluoroacetic acid 5% water and 5% triisopropyl silane for 2 hours. Cleavage solutions were collected and products precipitated by addition of cold diethyl ether. Solid products were isolated and the crude peptides were dissolved in DMSO (50 mM stock) based on average weights for each mixture. Libraries and single compounds were stored at −20° C. and further diluted to 10 mM stock plates for use in experiments.

### Synthesis of Y-Q(R,R)Eps and Y-G(R,R)Eps

All single component peptide epoxides were synthesized on the solid support using the protocols reported for DCG-04 (Greenbaum et al. (2000) Chem Biol 7: 569-581). The inhibitors were cleaved from the resin by addition of 90% trifluoroacetic acid 5% water and 5% triisopropyl silane for 2 hours. Ice cold ether (15 ml) was used to precipitate the products. The crude products were purified on a C₁₈ reverse phase HPLC column (Waters) using a linear gradient of 0-100% water-acetonitrile. Fractions containing the product were pooled, frozen and lyophilyzed to dryness. The identity of the product was confirmed by mass spectrometry. Electrospray mass spectrum: [M+H] calculated for: YG-( R,R)Eps C₁₇H₂₁N₃O₇ 380.1, found 380.1; YQ-(R,R)Eps C₂₀H₂₆N₄O₈ 451.2, found 451.2.

### Radiolabeling of Inhibitors

All compounds were iodinated and isolated using the protocol described by Bogyo et al. (2000) Chem Biol 7: 27-38.

### Preparation of Cell and Tissue Lysates

Tissues were dounce-homogenized in buffer A (50 mM Tris pH 5.5, 1 mM DTT, 5 mM MgC₁₂, 250 mM sucrose) and extracts centrifuged at 1,100×g for 10 min at 4° C. and the supernatant centrifuged at 22,000×g for 30 min at 4° C. Cells were homogenized using glass beads in buffer A and supernatants centrifuged for 15,000×g for 15 min at 4° C. The total protein concentration of the final supernatants (soluble) was determined by BCA protein quantification (Pierce).

### Labeling of Lysates with DCG-04, ¹²⁵I-DCG-04, ¹²⁵I-MB-074, ¹²⁵I-YQ-(R,R)Eps, Yellow-DCG- 04, Blue-DCG-04,Green DCG-04 or Red-DCG-04.

Lysates (100 mg total protein in 100 μL buffer; 50 mM Tris pH 5.5, 5 mM MgC12, 2 mM DTT) were labeled for 1 hour at 25° C. unless noted otherwise. DCG-04 was added to a final concentration of 10 mM. Equivalent amounts of all radioactive inhibitor stock solutions (approx. 10⁶ cpm per sample) were used for all labeling experiments. Fluorescent compounds were added to lysates to a final concentration of 0.1 mM. Samples were quenched by addition of 4×SDS sample buffer (for 1D SDS-PAGE) or by addition of solid urea to a final concentration of 9.5 M (for 2D SDS-PAGE). Fluorescent samples were analyzed using an ABI 377 DNA sequencer. Standard 15% SDS-PAGE gels of 0.4 mm thickness were prepared using 15 cm plates provided by the manufacturer. Samples were loaded and electrophoresed for 3-4 hrs at a constant current of 35 mA with voltage limited to 750 V. Gel images were created using the Gene Scan software provided by the manufacturer. In some experiments, fluorescent samples were analyzed by standard SDS-PAGE followed by scanning with a Molecular Dynamics Typhoon laser scanner.

### In situ Fluorescence Labeling

Dendritic cells (DC2.4) were plated on a 24-well dish (10₅ cells/well) embedded with sterile microscope cover slips, in RPMI medium containing 10% FBS. After 16 hours, cells were washed with 1 ml TC-199 medium and incubated with 1 mM of Bodipy-DCG-04 in TC-199 for 12 hours at 37° C. Cells were washed 3 times with 1 ml TC-199 and incubated for 5 hr in probe-free medium. Subsequently, cells were either lysed in buffer A and analyzed on a 12.5% SDS-PAGE using a fluorescent scanner or viewed under a fluorescent microscope.

### Gel Electrophoresis

One-dimensional SDS-PAGE and two-dimensional IEF was performed as described by Bogyo et al. (1998) Chem Biol 5: 307-320.

### Competition Labeling and Analysis of Data

Rat liver lysates (100 mg total protein in 100 μL buffer A; 50 mM Tris pH 5.5, 5 mM MgCl₂, 2 mM DTT) or purified cathepsins (1 μg protein in 100 μL buffer A) were pre-incubated with 10 μM of each library member (diluted from 10 mM DMSO stocks) for 30 min at room temperature. Samples were then labeled by addition of ¹²⁵I-DCG-04 to each sample followed by further incubation at room temperature for 1 hour. Samples were quenched by the addition of 4×sample buffer, resolved by SDS-PAGE, and analyzed by PhosporImaging (Molecular Dynamics). Bands corresponding to each labeled protease were quantitated. Inhibitor treated samples were compared to an untreated control sample. Numerical values for percent written by Eisen and co-workers (Eisen et al. (1998) μProc. Natl. Acad. Sci., USA, 95: 14863-14868). These programs can be obtained from www.microarrays.org.

### Purification and Identification of Affinity Labeled Proteases from Rat Liver

Protein lysates prepared in buffer A (50 mM Acetate buffer, 5 mM DTT, 0.1% Triton X-100) were incubated with 5 mM DCG-04 for 1.5 hours at room temperature. After incubation the protein lysate was passed through a PD10 column pre-equilibrated with buffer B (50 mM Tris-Base 7.4, 150 mM NaCl) and proteins were eluted with the same buffer. SDS was added to eluted proteins to a final concentration of 0.5% and the solution boiled for 10 minutes, diluted 2.5 fold with buffer B (to reduce SDS concentration to 0.2%) and incubated with 100 ml bed volume of pre-washed Streptavidin beads for 1 hour at room temperature. Beads were washed 5 times with buffer B, and bound proteins were eluted by boiling for 10 minutes in the presence of 100 ml SDS sample buffer. For 2D analysis, samples in SDS sample buffer were diluted 1:1 with IEF sample buffer (9.5 M, 5% BME, 2% NP-40, 1.6% pH 5-7 ampholines and 0.4% pH 3.5-10 ampholines) and pure NP-40 was added (25% of volume of sample). Samples were applied to IEF tube gels and electrophoresed at 1000V for 13 hours followed by separation in the second dimension on 15% SDS-PAGE gels. The resulting gels were fixed in 12% acetic acid/50% methanol stained with silver according to reported protocols (Bogyo et al. (1998) μChem Biol 5: 307-320). Spots were excised, digested with trypsin, and fractionated by reversed-phase HPLC on an Ultimate system, equipped with a FAMOS auto-injector (LC Packings, San Francisco, Calif.). Experimental conditions were: 1 mL injection; 75 mmx150 mm PepMap column; solvent A, H₂O with 0.1% formic acid; solvent B, acetonitrile with 0.1 % formic acid; gradient, 0-30% B in 40 min at a flow rate of ˜250 nL/min. Mass spectrometry detection was performed on a QSTAR quadrupole-orthogonal- acceleration-time-of-flight tandem mass spectrometer (Applied Biosystems/MDS Sciex, Foster City, Calif.) in information dependent acquisition (IDA) mode: 2 second survey acquisitions were followed by 5 second CID acquisitions, in which the most abundant ion of each survey scan was selected as the precursor. All the singly charged ions as well as some trypsin autolysis products were excluded from the precursor ion selection. The collision energy was optimized and adjusted automatically depending on the charge state and the m/z value of the precursor ions selected. The mass range recorded in survey acquisitions was m/z 300-1400. For CID experiments the lower mass limit was changed to m/z 60. All the data were measured using a two point external calibration. The instrument affords ˜8000 resolution and 30 ppm mass accuracy with external calibration in both MS and CID mode. Proteins were identified automatically by Mascot database search using the MS/MS data (Matrix Science Ltd., London, UK).

### Example 3

### Uses of Fluorescently Labeled Probes

FIG. 16 illustrates the screening of small molecule libraries against the complete set of papain family cysteine proteases in Rat liver. Total protein extracts from rat liver were incubated with positional scanning libraries of small molecules based on the epoxide probe structure. After 30 minutes pre-incubation with inhibitors, samples treated with compounds 1-20 were labeled with Green-DCG-04. Samples treated with compounds 21-40 were labeled with Blue-DCG-04, and samples treated with compounds 41-60 were labeled with Yellow-DCG-04. After one hour labeling the samples were quenched by addition of SDS sample buffer. The yellow, blue, and green samples were mixed and a small portion was analyzed by SDS-PAGE and laser scanning on an ABI 377 DNA sequencer. This image shows a typical gel image generated from scanning of the gel as well as the process by which labeled bands can be quantitated (panel to left). Small molecules can be analyzed for their potency and selectivity for targets in the rat liver proteasome using this method. Note that each color data can be separately extracted due to non-overlapping emission spectra of the chosen fluorophores. This approach therefore allows analysis of up to 80 samples in a single gel using four color labels.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A system for identifying papain cysteine hydrolases comprising the use in combination of at least two compounds of the formula:
A-L¹-Hy-L²-E
where:
A is at least 15 Dal and not more than about 2 kDal and is a ligand;
L¹ and L² may be the same or different and are a bond or a chain of from 1 to 40, atoms;
Hy is a hydrophobic group that specifically binds in the papain cysteine protease pocket.; and
E is an epoxide that covalently bonds to the active site of the papain cysteine hydrolase.

2. A system according to claim 1, wherein A is a detectable ligand.

3. A system according to claim 2, wherein said detectable ligand is a fluorescer.

4. A system according to claim 1, wherein A is a ligand that binds to a naturally occurring receptor.

5. A system according to claim 1, wherein Hy is an aliphatic, aromatic or alicyclic side chain bonded to a carbon chain linking an amino group to a carboxy group.

6. A system according to claim 1, wherein each of said compounds has a radioactive label.

7. A compound of the formula: 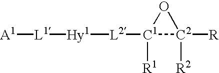
wherein:
A¹ is a moiety that provides a detectable signal or a ligand;
L^{1′} and L^{2′} are the same or different and are a bond or an aliphatic chain of from 1 to 8 carbon atoms joined to A^{1′} or the epoxide C¹ annular carbon atom and Hy¹ through the same or different functional groups;
Hy¹ is a neutral hydrophobic amino acid having a total of at least 4 and not more than about 20 carbon atoms, having a side chain of at least about 2 carbon atoms and lacking a quaternary carbon atom;
the R groups are the same or different there being not more than two of the R groups other than hydrogen, where the total number of carbon atoms for all of the R groups is from 0 to 8.

8. A compound according to claim 7, wherein A¹ is a fluorescer.

9. A compound according to claim 7, wherein said epoxide is a single stereoisomer.

10. A compound according to claim 7, wherein A¹ is a ligand.

11. A compound according to claim 7, wherein Hy¹ comprises a carbocyclic side chain.

12. A compound according to claim 7, wherein Hy¹ comprises an acyclic aliphatic side chain.

13. A cell comprising a papain cysteine hydrolase bonded to an hydroxyethylene group of an epoxide compound as a result of a reaction between said papain cysteine hydrolase and an annular carbon atom of said epoxide compound, said epoxide compound of the formula:
A-L¹-Hy-L²-E
wherein:
A is at least 15 Dal and not more than about 2 kDal and is a ligand;
L¹ and L² may be the same or different and are a bond or a chain of from 1 to 40, atoms;
Hy is a hydrophobic group that specifically binds in the papain cysteine protease pocket; and
E is an epoxide.

14. A cell according to claim 13, wherein said ligand is a fluorescer.

15. A cell according to claim 13, wherein said epoxide compound comprises a radioactive label.

16. A method for determining the presence of at least one active papain cysteine hydrolase target in a sample, said method comprising:
combining said sample with at least one compound of the formula:
A-L¹-Hy-L²-E
where:
A is at least 15 Dal and not more than about 2 kDal and is a ligand;
L¹ and L² may be the same or different and are a bond or a chain of from 1 to 40, atoms;
Hy is a hydrophobic group that specifically binds in said papain cysteine protease pocket.; and
E is an epoxide that covalently bonds to the active site of the papain cysteine hydrolase under conditions wherein said papain cysteine hydrolase target reacts with said epoxide to form a covalently linked conjugate; and
determining the presence of said papain cysteine hydrolase by means of said ligand.

17. A method according to claim 16, wherein said papain cysteine hydrolase and said compound are present in a cell.

18. A method according to claim 16, wherein said compound comprises a radioactive label or said ligand is a fluorescer label and said determining comprises detecting said label.

19. A method according to claim 16, including the additional step of sequestering said covalently linked conjugate by means of said ligand.

20. A method according to claim 16, wherein a plurality of compounds are combined, each of said compounds having a different profile of binding to papain cysteine hydrolases.

21. A probe for monitoring or identifying cysteine hydrolase activity, said probe comprising a compound having the formula:
A-L¹-(aa¹)ᵢ-(aa²)ⱼ-(aa³)ₖ-(aa⁴)ₗ-L²ₘ-E
wherein A is a ligand or a detectable label;
L¹ is a linker;
L², when present, is a linker;
aa¹, aa², aa³, and aa⁴, when present, are independently selected amino acids;
i, j, k, l, and m are independently 0 or 1;
E is an electrophile; and
at least two of aa¹, aa², aa³, and aa⁴ are present.

22. The probe of claim 21, wherein A is a detectable label.

23. The probe of claim 21, wherein A is a fluorescent label.

24. The probe of claim 21, wherein at least one of aa¹, aa², aa³, and aa⁴ is labeled with a detectable label.

25. The probe of claim 24, wherein A is a ligand.

26. The probe of claim 24, wherein said detectable label is a radioactive label selected from the group consisting of ³H, ¹²⁵I, ³⁵S, ¹⁴C, and ³²P.

27. The probe of claim 21, wherein aa¹, aa², aa³, and aa⁴, when present, are independently selected from the group consisting of alanine, valine, leucine, isoleucine, proline, phenylalanine, tryptophan, methionine, aspartic acid, glutamic acid, lysine, arginine, histidine, glycine, serine, threonine, cysteine, tyrosine, asparagine, glutamine, and norleucine.

28. The probe of claim 21, wherein said electrophile is selected from the group consisting of a diazomethyl ketone, a fluoromethyl ketones, an acyloxymethyl ketone, a chloromethyl ketone, an o-acylhydroxylamine, a vinyl sulfone, an epoxysuccinic derivative, and an epoxide.

29. The probe of claim 21, wherein A is an affinity tag.

30. The probe of claim 29, wherein A is an affinity tag is selected from the group consisting of a biotin, an avidin, a streptavidin, an antibody, and an epitope tag.

31. The probe of claim 30, wherein A is an epitope tag selected from the group consisting of a polyhistidine, a polyarginine, a Flag-tag, an HA-tag, a myc-tag, and a DYKDDDDK epitope.

32. The probe of claim 21, wherein L¹ and L², when present, are independently selected from the group consisting of a straight chain carbon linker, a branched-chain carbon linkers, a cleavable linker, and a heterocyclic carbon linker.

33. The probe of claim 32, wherein L¹ and L², when present, are independently selected straight chain C₁ to C₂₀ carbon linkers.

34. The probe of claim 32, wherein L¹ is a hexanoic acid linker.

35. The probe of claim 32, wherein L¹ is a photolabile cleavable linker or an oxidizable cleavable linker.

36. The probe of claim 21, wherein:
i and j are zero; and
k and l are 1.

37. The probe of claim 36, wherein:
aa³ is (tyrosine); and
aa⁴ is selected from the group consisting of alanine, valine, leucine, isoleucine, proline, phenylalanine, tryptophan, methionine, aspartic acid, glutamic acid, lysine, arginine, histidine, glycine, serine, threonine, cysteine, tyrosine, asparagine, glutamine, and norleucine.

38. The probe of claim 37, wherein L¹ is an amino hexanoic acid spacer and A is a biotin.

39. The probe of claim 38, wherein said E is an epoxide.

40. The probe of claim 21, wherein said probe comprises the formula:

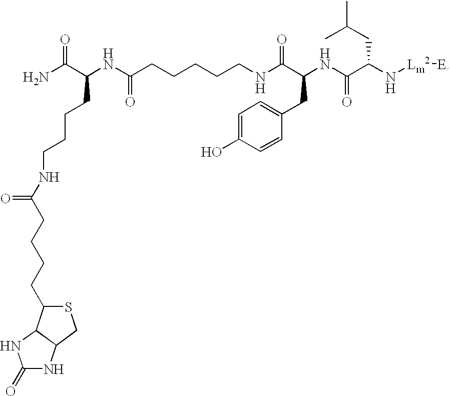

41. The probe of claim 21, wherein said probe comprises the formula:

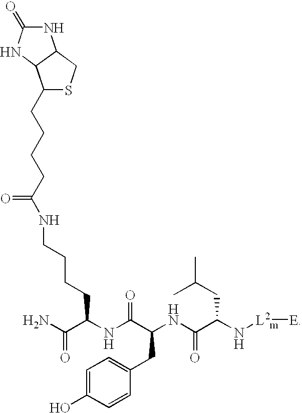

42. The probe of claim 21, wherein said probe comprises the formula:

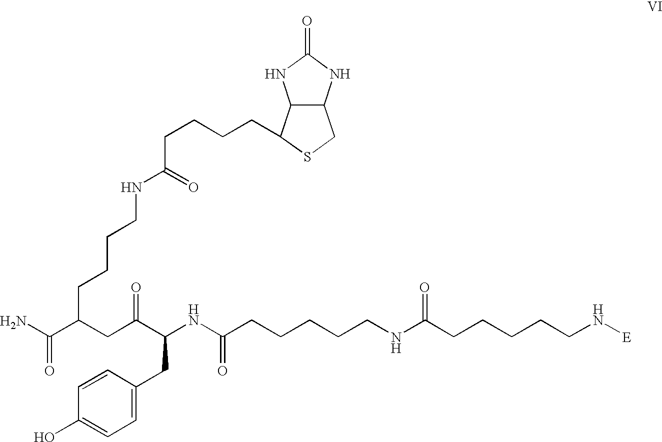

43. The probe of claim 21, wherein said probe comprises the formula of BODEPY558/568-DCG-04.

44. The probe of claim 21, wherein said probe comprises the formula of BODIPY493/503-DCG-04.

45. The probe of claim 21, wherein said probe comprises the formula of BODIPY530/550-DCG-04.

46. The probe of claim 21, wherein said probe comprises the formula of BODEPY588/616-DCG-04.

47. The probe of claim 21, wherein said compound is selected from the group consisting of DCG-01, DCG-04, and DCG-03.

48. The probe of claim 21, wherein said probe is attached to a solid support.

49. The probe of claim 48, wherein said probe is attached to a solid support A where A is a ligand.

50. A probe library for monitoring or identifying cysteine protease activity, said probe library comprising a plurality of members each member of said plurality of members comprising a compound having the formula:
A-L¹-(aa¹)ᵢ-(aa²)ⱼ-(aa³)ₖ-(aa⁴)ₗ-L²ₘ-E
wherein A is a ligand or a detectable label;
L¹ is a linker;
L², when present, is a linker;
aa¹, aa², aa³, and aa⁴, when present, are independently selected amino acids;
i, j, k, l, and m are independently 0 or 1;
E is an electrophile; and
at least two of aa¹, aa², aa³, and aa⁴ are present.

51. The probe library of claim 50, wherein A is a detectable label.

52. The probe library of claim 51, wherein A is a fluorescent label.

53. The probe library of claim 50, wherein at least one of aa¹, aa², aa³, and aa⁴ is labeled with a detectable label.

54. The probe library of claim 53, wherein A is a ligand.

55. The probe library of claim 53, wherein said detectable label is a radioactive label selected from the group consisting of ³H, ¹²⁵I, ³⁵S, ¹⁴C, and ³²P.

56. The probe library of claim 50, wherein aa¹, aa², aa³, and aa⁴, when present, are independently selected from the group consisting of alanine, valine, leucine, isoleucine, proline, phenylalanine, tryptophan, methionine, aspartic acid, glutamic acid, lysine, arginine, histidine, glycine, serine, threonine, cysteine, tyrosine, asparagine, glutamine, and norleucine.

57. The probe library of claim 50, wherein said electrophile is selected from the group consisting of a diazomethyl ketone, a fluoromethyl ketone, an acyloxymethyl ketone, a chloromethyl ketone, an o-acylhydroxylamine, a vinyl sulfone, an epoxysuccinic derivative, and an epoxide.

58. The probe library of claim 50, wherein A is an affinity tag.

59. The probe library of claim 58, wherein A is an affinity tag selected from the group consisting of a biotin, an avidin, a streptavidin, an antibody, and an epitope tag.

60. The probe library of claim 59, wherein A is an affinity that that is an epitope tag selected from the group consisting of a polyhistidine, a polyarginine, a Flag-tag, an HA-tag, a myc-tag, and a DYKDDDDK epitope.

61. The probe library of claim 50, wherein L¹ and L², when present, are independently selected from the group consisting of a straight chain carbon linker, a branched-chain carbon linkers, and a heterocyclic carbon linker.

62. The probe library of claim 61, wherein L¹ and L², when present, are independently selected straight chain C₁ to C₂₀ carbon linkers.

63. The probe library of claim 61, wherein L¹ is a hexanoic acid linker.

64. The probe library of claim 50, wherein:
i and j are zero; and
k and l are 1.

65. The probe library of claim 64, wherein:
aa³ is (tyrosine) and
aa⁴ is selected from the group consisting of alanine, valine, leucine, isoleucine, proline, phenylalanine, tryptophan, methionine, aspartic acid, glutamic acid, lysine, arginine, histidine, glycine, serine, threonine, cysteine, tyrosine, asparagine, glutamine, and norleucine.

66. The probe library of claim 65, wherein L¹ is an amino hexanoic acid spacer and A is a biotin.

67. The probe library of claim 66, wherein E is an epoxide.

68. The probe of claim 50, wherein said compound has the formula:

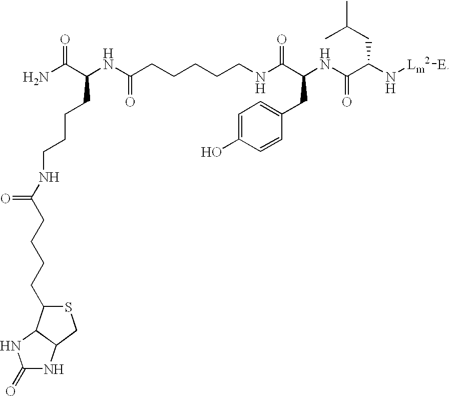

69. The probe of claim 50, wherein said compound has the formula:

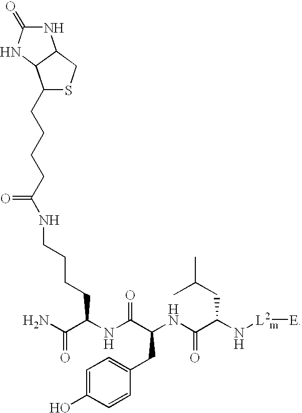

70. The probe of claim 50, wherein said compound has the formula:

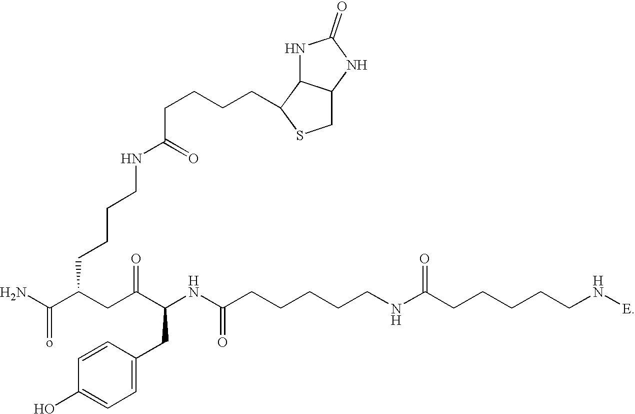

71. The probe library of claim 50, wherein said probe comprises the formula of BODIPY558/568-DCG-04.

72. The probe library of claim 50, wherein said probe comprises the formula of BODIPY493/503-DCG-04.

73. The probe library of claim 50, wherein said probe comprises the formula of BODIPY530/550-DCG-04.

74. The probe library of claim 50, wherein said probe comprises the formula of BODIPY588/616-DCG-04.

75. The probe library of claim 50, wherein said library comprises at least 10 different members.

76. The probe library of claim 50, wherein said library comprises at least 20 different members.

77. The probe library of claim 50, wherein said compounds are attached to a solid support.

78. The probe library of claim 77, wherein the compounds are attached to a solid support through said affinity tag.

79. A method of identifying or determining activity of a cysteine hydrolase, said method comprising:
i) providing a biological sample;
ii) contacting said biological sample with a compound having the formula:
A-L¹-(aa¹)ᵢ-(aa²)ⱼ-(aa³)ₖ-(aa⁴)ₗ-L²ₘ-E
wherein A is a ligand or a detectable label;
L¹ is a linker;
L², when present, is a linker;
aa¹, aa², aa³, and aa⁴, when present, are independently selected amino acids;
i, j, k, l, and m are independently 0 or 1;
E is an electrophile; and
at least two of aa¹, aa², aa³, and aa⁴ are present; and
iii) detecting specific binding of said compound to a component of said biological sample whereby said detecting identifies or quantifies a cysteine hydrolase.

80. The method of claim 79, wherein A is a detectable label.

81. The method of claim 80, wherein A is a fluorescent label.

82. The method of claim 79, wherein at least one of aa¹, aa², aa³, and aa⁴ is labeled with a detectable label.

83. The method of claim 82, wherein said A is a ligand.

84. The method of claim 82, wherein said detectable label is a radioactive label selected from the group consisting of ³H, ¹²⁵I, ³⁵S, ¹⁴C, and ³²P.

85. The method of claim 79, wherein said biological sample comprises a crude cellular extract.

86. The method of claim 79, wherein said biological sample comprises a purified protein.

87. The method of claim 84, wherein said detecting comprises detecting direct labeling of said component by detecting the label on said compound.

88. The method of claim 79, wherein said method further comprises contacting said biological sample with a known inhibitor of a cysteine hydrolase and determining the amount of binding of said compound competed by said inhibitor of a cysteine hydrolase.

89. The method of claim 79, wherein said detecting comprises contacting a control comprising a denatured biological sample with said compound and detecting the differences between the binding of said compound to said sample and said compound to said control.

90. The method of claim 79, wherein said detecting further comprises isolating a component specifically bound by said compound by contacting said compound with a ligand that binds to said affinity tag.

91. The method of claim 90, wherein said affinity tag is a biotin and said ligand is a streptavidin or a modified streptavidin.

92. The method of claim 90, wherein said affinity tag is a poly-His tag and said ligand is a Ni-NTA.

93. The method of claim 90, wherein said method further comprises digesting the component bound by said compound.

94. The method of claim 90, wherein said method further comprises performing an amino acid analysis of the component bound by said compound.

95. The method of claim 90, wherein said method further comprises performing mass spectroscopy of the component bound by said compound.

96. The method of claim 79, wherein said biological sample is a crude cellular extract and the binding profile of said probe is compared to binding profiles stored in a specificity fingerprint database to identify a protease in said extract.

97. The method of claim 79, wherein said detecting comprises comparing a binding profile of said probe to one or more components of said sample to the binding profile of the members of said library to one or more components of a second sample.

98. The method of claim 79, wherein said detecting comprises comparing specific binding of the compound to a component of the biological sample with the binding of the compound to one or more components of a sample from a different cell or tissue.

99. The method of claim 98, wherein said biological sample is a sample from a pathological or diseased cell or tissue and said different cell or tissue is a healthy cell or tissue.

100. The method of claim 79, wherein said compound is a member of a library of cysteine hydrolase probes comprising a plurality of different cysteine hydrolase probes and said contacting comprises contacting said biological sample with said library.

101. The method of claim 100, wherein said biological sample is a purified protease and binding of each member of said library to said protease is recorded to produce a specificity fingerprint for said protease.

102. The method of claim 101, wherein said specificity fingerprint is entered into a database of specificity fingerprints for various proteases.

103. The method of claim 100, wherein said biological sample is a crude cellular extract and the binding profile of the members of said library is compared to binding profiles stored in a specificity fingerprint database to identify a protease in said extract.

104. The method of claim 100, wherein said detecting comprises comparing a binding profile of the members of said library to one or more components of said sample to the binding profile of the members of said library to one or more components of a second sample.

105. The method of claim 100, wherein said detecting comprises comparing a binding profile of the members of said library to one or more components of the biological sample with the a binding profile of the members of said library to one or more components of a sample from a different cell or tissue.

106. The method of claim 105, wherein said biological sample is a sample from a pathological or diseased cell or tissue and said different cell or tissue is a healthy cell or tissue.

107. A method of identifying an agent that modulates activity of a cysteine hydrolase, said method comprising:
i) providing a biological sample;
ii) contacting said biological sample with a compound having the formula:
A-L¹-(aa¹)ᵢ-(aa²)ⱼ-(aa³)ₖ-(aa⁴)ₗ-L²ₘ-E
wherein A is a ligand or a detectable label;
L¹ is a linker;
L², when present, is a linker;
aa¹, aa², aa³, and aa⁴, when present, are independently selected amino acids;
i, j, k, l, and m are independently 0 or 1;
E is an electrophile; and
at least two of aa¹, aa², aa³, and aa⁴ are present;
iii) contacting said biological sample with a test agent; and
iv) detecting specific binding of said compound to a component of said biological sample whereby a difference in the binding of said compound to a component of said biological sample as compared to the binding of said compound to a component of said biological sample where said test agent is absent or present at a lower concentration indicates that said test agent modulates activity of said cysteine hydrolase.

108. The method of claim 107, wherein A is a detectable label.

109. The method of claim 108, wherein A is a fluorescent label.

110. The method of claim 107, wherein at least one of aa¹, aa², aa³, and aa⁴ is labeled with a detectable label.

111. The method of claim 107, wherein A is a ligand.

112. The method of claim 107, wherein said detectable label is a radioactive label selected from the group consisting of ³H, ¹²⁵I, ³⁵S, ¹⁴C, and ³²P.

113. The method of claim 107, wherein said biological sample comprises a crude cellular extract.

114. The method of claim 107, wherein said biological sample comprises a purified protein.

115. The method of claim 114, wherein said detecting comprises detecting direct labeling of said component by detecting the label on said compound.

116. The method of claim 107, wherein said detecting comprises contacting a control comprising a denatured biological sample with said compound and detecting the differences between the binding of said compound to said sample and said compound to said control.

117. The method of claim 107, wherein said compound is a member of a library of said compounds comprising a plurality of different compounds and said contacting comprises contacting said biological sample with said library.

118. The method of claim 107, wherein said detecting further comprises isolating a component specifically bound by said compound by contacting said compound with a ligand that binds to said affinity tag.

119. The method of claim 118, wherein said affinity tag is a biotin and said ligand is a streptavidin or a modified streptavidin.

120. The method of claim 107, wherein said biological sample is a purified protease and binding of each member of said library to said protease is recorded to produce a modulation fingerprint for said test agent.

121. The method of claim 120, wherein said specificity fingerprint is entered into a database of modulation fingerprints for various agents.

122. The method of claim 107, wherein said biological sample is a crude cellular extract and the pattern of binding of the members of said library is compared to patterns of binding stored in a specificity fingerprint database to classify a test agent's mode of activity.

123. The method of claim 107, wherein said detecting comprises comparing specific binding of the compound to a component of the biological sample with the binding of the compound to a component of a sample from a different cell or tissue.

124. A method of synthesizing an inhibitor of a cysteine protease said method comprising:
synthesizing an oligopeptide in a solid phase peptide synthesis procedure;
coupling a (2s,3s)-oxirane-2,3-dicarboxylate to said oligopeptide; and
cleaving said peptide from said solid support to produce an oligopeptide bearing an epoxide.

125. The method of claim 124, wherein said oligopeptide is a dipeptide.

126. The method of claim 124, wherein said cleaving uses trifluoroacetic acid (TFA).

127. A kit for monitoring or identifying cysteine hydrolase activity, said kit comprising a container containing a compound having the formula:
A-L¹-(aa¹)ᵢ-(aa²)ⱼ-(aa³)ₖ-(aa⁴)ₗ-L²ₘ-E
wherein A is a ligand or a detectable label;
L¹ is a linker;
L², when present, is a linker;
aa¹, aa², aa³, and aa⁴, when present, are independently selected amino acids;
i, j, k, l, and m are independently 0 or 1;
E is an electrophile; and
at least two of aa¹, aa², aa³, and aa⁴ are present.

128. The kit of claim 127, wherein A is a detectable label.

129. The kit of claim 128, wherein A is a fluorescent label.

130. The kit of claim 127, wherein at least one of aa¹, aa², aa³, and aa⁴ is labeled with a detectable label.

131. The kit of claim 130, wherein A is a ligand.

132. The kit of claim 130, wherein said detectable label is a radioactive label selected from the group consisting of ³H, ¹²⁵I, ³⁵S, ¹⁴C, and ³²P.

133. The kit of claim 127, further comprising a known inhibitor of a cysteine hydrolase.

134. The kit of claim 127, wherein said compound is a member of a library of cysteine hydrolase probes comprising a plurality of different cysteine hydrolase probes and said kit comprises said library.

135. The kit of claim 127, wherein said kit further comprises instructional materials providing protocols for using a cysteine hydrolase probe to monitor or identify cysteine hydrolase activity or to isolate a cysteine hydrolase.


SEQUENCE LISTING (2 sequences)

SEQ ID NO: 1 - protein, 8 aa, Artificial Sequence
  epitope
DYKDDDDK

SEQ ID NO: 2 - protein, 12 aa, Rattus rattus
  misc_feature (9)..(9): Xaa is L or I
MGEDSYPYXIGK